(19) United States
(12) Patent Application Publication
(10) Pub. No.: US 2020/0376146 A1
(43) Pub. Date: Dec. 3, 2020

(54) NANOBIOLOGIC COMPOSITIONS FOR INHIBITING TRAINED IMMUNITY

(71) Applicants: ICAHN SCHOOL OF MEDICINE, New York, NY (US); STICHTING KATHOLIEKE UNIVERSITEIT, Numegen (NL)

(72) Inventors: WILLEM MULDER, NEW YORK, NY (US); JORDI OCHANDO, NEW YORK, NY (US); ZAHI FAYAD, NEW YORK, NY (US); RAPHAEL DUIVENVOORDEN, NEW YORK, NY (US); ABRAHAM TEUNISSEN, NEW YORK, NY (US); CARLOS PEREZ-MEDINA, NEW YORK, NY (US); MIHAI NETEA, NIJMEGEN (NL); LEO JOOSTEN, NIJMEGEN (NL)

(21) Appl. No.: 16/863,333

(22) Filed: Apr. 30, 2020

Related U.S. Application Data
(63) Continuation of application No. PCT/US18/61939, filed on Nov. 20, 2018.
(60) Provisional application No. 62/588,790, filed on Nov. 20, 2017.
(60) Provisional application No. 62/734,664, filed on Sep. 21, 2018.

(51) Int. Cl. A61K 51/04; A61K 51/12; A61K 51/08

(52) U.S. Cl. CPC A61K 51/0408; A61K 51/0497; A61K 45/06; A61K 51/08; A61K 51/0493; A61K 51/1227

(57) ABSTRACT

The invention relates to therapeutic nanobiologic compositions and methods of treating patients who have had an organ transplant, or who suffer from atherosclerosis, arthritis, inflammatory bowel disease including Crohn's, autoimmune diseases including diabetes, and/or autoinflammatory conditions, or after a cardiovascular events, including stroke and myocardial infarction, by inhibiting trained immunity, which is the long-term increased responsiveness, the result of metabolic and epigenetic re-wiring of myeloid cells and their stem cells and progenitors in the bone marrow and spleen and blood induced by a primary insult, and characterized by increased cytokine excretion after re-stimulation with one or multiple secondary stimuli.

### CROSS-REFERENCE TO RELATED APPLICATIONS

This application is a continuation and claims priority benefit under 35 USC 365(c) to international application PCT/US18/61939 filed Nov. 20, 2018, which claims priority benefit to U.S. patent application 62/588,790 filed Nov. 20, 2017 and U.S. patent application 62/734,664 filed Sep. 21, 2018, the entirety of which are all incorporated herein by reference.

### STATEMENT REGARDING FEDERALLY SPONSORED R&D

This invention was made with government support under grant R01 HL118440 awarded by the National Institutes of Health. The government has certain rights in the invention.

### FIELD OF THE INVENTION

The invention relates to therapeutic nanobiologic compositions and methods of treating patients who have had an organ transplant, or who suffer from atherosclerosis, arthritis, inflammatory bowel disease including Crohn's, autoimmune diseases, and/or autoinflammatory conditions, or after a cardiovascular events, including stroke and myocardial infarction, by inhibiting trained immunity, which is a secondary long-term hyper-responsiveness, as manifested by increased cytokine excretion caused by metabolic and epigenetic rewiring, to re-stimulation after a primary insult of myeloid cells and their progenitors and stem cells in the bone marrow, spleen and blood.

### BACKGROUND OF THE INVENTION

Current treatments for patients who suffer from autoimmune and immune system dysfunction are inadequate. Patients who have had an organ transplant, or who suffer from atherosclerosis, arthritis, inflammatory bowel disease including Crohn's, autoimmune diseases including diabetes, and/or autoinflammatory conditions, or after cardiovascular events, including stroke and myocardial infarction, are in need of a treatment paradigm that is durable, and that does not cause more problems in side effects than the primary treatment itself.

### SUMMARY OF THE INVENTION

Accordingly, to address these and other deficiencies in the prior art, in a preferred embodiment of the invention, there is provided a method of treating a patient in need thereof with a therapeutic agent for inhibiting trained immunity.

Trained Immunity is defined by a secondary long-term hyper-responsiveness, as manifested by increased cytokine excretion caused by metabolic and epigenetic rewiring, to re-stimulation after a primary insult of myeloid cells and their progenitors and stem cells in the bone marrow, spleen and blood. Trained Immunity (also called innate immune memory) is also defined by a long-term increased responsiveness (e.g. high cytokine production) after re-stimulation with a secondary stimulus of myeloid innate immune cells, being induced by a primary insult stimulating these cells or their progenitors and stem cells in the bone marrow and spleen, and mediated by epigenetic, metabolic and transcriptional rewiring.

### Treating a Patient Affected by Trained Immunity

In a non-limiting preferred embodiment of the invention, there is provided a method of treating a patient affected by trained immunity to reduce in said patient an innate immune response, comprising:

administering to said patient a nanobiologic composition in an amount effective to reduce a hyper-responsive innate immune response,
wherein the nanobiologic composition comprises (i) a nanoscale assembly, having (ii) an inhibitor drug incorporated in the nanoscale assembly,
wherein the nanoscale assembly is a multi-component carrier composition comprising: (a) phospholipids, and,
(b) apolipoprotein A-I (apoA-I) or a peptide mimetic of apoA-I,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell,
wherein the nanoscale assembly delivers the drug to myeloid cells, myeloid progenitor cells or hematopoietic stem cells in bone marrow, blood and/or spleen of the patient,
and whereby in the patient the hyper-responsive innate immune response caused by trained immunity is reduced.

In a non-limiting preferred embodiment of the invention, there is provided a method of treating a patient affected by trained immunity to reduce in said patient an innate immune response, wherein the nanoscale assembly is a multi-component carrier composition comprising:

phospholipids,
apolipoprotein A-I (apoA-I) or a peptide mimetic of apoA-I, and
a hydrophobic matrix comprising one or more triglycerides, fatty acid esters, hydrophobic polymers, or sterol esters, or a combination thereof.

In another non-limiting preferred embodiment of the invention, there is provided a method of treating a patient affected by trained immunity to reduce in said patient a hyper-responsive innate immune response, wherein the nanoscale assembly is a multi-component carrier composition comprising:

phospholipids,
apolipoprotein A-I (apoA-I) or a peptide mimetic of apoA-I,
a hydrophobic matrix comprising one or more triglycerides, fatty acid esters, hydrophobic polymers, or sterol esters, or a combination thereof, and
cholesterol.

### Promoting Allograft Acceptance

In a non-limiting preferred embodiment of the invention, there is provided a method of promoting allograft acceptance in a patient that is a transplant recipient, comprising:

administering to said patient a nanobiologic composition in an amount effective to induce permanent allograft acceptance,
wherein the nanobiologic composition comprises (i) a nanoscale assembly, having (ii) an inhibitor drug incorporated in the nanoscale assembly,
wherein the nanoscale assembly is a multi-component carrier composition comprising: (a) a phospholipid or a mixture of phospholipids, and,
(b) apolipoprotein A-I (apoA-I) or a peptide mimetic of apoA-I,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell,
wherein the nanoscale assembly delivers the drug to myeloid cells, myeloid progenitor cells or hematopoietic stem cells in bone marrow, blood and/or spleen of the patient,
and whereby permanent allograft acceptance is induced in the transplant recipient patient.

In a non-limiting preferred embodiment of the invention, there is provided a method of promoting allograft acceptance in a patient that is a transplant recipient, wherein the nanoscale assembly is a multi-component carrier composition comprising:

a phospholipid or a mixture of phospholipids,
apolipoprotein A-I (apoA-I) or a peptide mimetic of apoA-I, and
a matrix lipid selected from one or more triglycerides, fatty acid esters, hydrophobic polymers, and sterol esters.

In a non-limiting preferred embodiment of the invention, there is provided a method of promoting allograft acceptance in a patient that is a transplant recipient, wherein the nanoscale assembly is a multi-component carrier composition comprising:

a phospholipid or a mixture of phospholipids,
apolipoprotein A-I (apoA-I) or a peptide mimetic of apoA-I,
a matrix lipid selected from one or more triglycerides, fatty acid esters, hydrophobic polymers, and sterol esters, and
cholesterol.

### Durable Effect

In a non-limiting preferred embodiment of the invention, there is provided in any one of methods herein, wherein the hyper-responsive innate immune response is reduced for at least 7 to 30 days.

In a non-limiting preferred embodiment of the invention, there is provided in any one of methods herein, wherein the hyper-responsive innate immune response is reduced for at least 30 to 100 days.

In a non-limiting preferred embodiment of the invention, there is provided in any one of methods herein, wherein the long-term hyperresponsiveness of myeloid cells, their stem cells and progenitors as a result of trained immunity (hyper-responsive innate immune response) is reduced for at least 100 days up to several years.

In a non-limiting preferred embodiment of the invention, there is provided in any one of methods herein, wherein the nanobiologic composition is administered once and wherein the long-term hyperresponsiveness of myeloid cells, their stem cells and progenitors as a result of trained immunity is reduced for at least 30 days.

In a non-limiting preferred embodiment of the invention, there is provided in any one of methods herein, wherein the nanobiologic composition is administered at least once per day in each day of a multiple-dosing regimen, and wherein the long-term hyperresponsiveness of myeloid cells, their stem cells and progenitors as a result of trained immunity is reduced for at least 30 days.

In a non-limiting preferred embodiment of the invention, there is provided in any one of methods herein, wherein trained Immunity is defined by a secondary long-term hyper-responsiveness, as manifested by increased cytokine excretion caused by metabolic and epigenetic rewiring, to re-stimulation after a primary insult of myeloid cells and their progenitors and stem cells in the bone marrow, spleen and blood.

In a non-limiting preferred embodiment of the invention, there is provided in any one of methods herein, wherein trained immunity is defined by a long-term increased responsiveness from high cytokine production after re-stimulation with a secondary stimulus of myeloid innate immune cells, being induced by a primary insult stimulating these cells or their progenitors and stem cells in the bone marrow, and mediated by epigenetic, metabolic and transcriptional rewiring.

### Diseases, Disorders, and Conditions

In a non-limiting preferred embodiment of the invention, there is provided in any one of methods herein, wherein the patient affected by trained immunity is a recipient of an organ transplant, or suffers from atherosclerosis, arthritis, inflammatory bowel disease including Crohn's, an autoimmune disease including diabetes, an autoinflammatory condition, or has suffered a cardiovascular event, including stroke and myocardial infarction.

In a non-limiting preferred embodiment of the invention, there is provided in any one of methods herein, wherein the patient is a transplant recipient, or suffers from atherosclerosis, arthritis, or inflammatory bowel disease, or has suffered a cardiovascular event.

In a non-limiting preferred embodiment of the invention, there is provided in any one of methods herein, wherein the patient has undergone a transplant and the transplanted tissue is lung tissue, heart tissue, kidney tissue, liver tissue, retinal tissue, corneal tissue, skin tissue, pancreatic tissue, intestinal tissue, genital tissue, ovary tissue, bone tissue, tendon tissue, bone marrow, or vascular tissue.

In a non-limiting preferred embodiment of the invention, there is provided in any one of methods herein, wherein the method is performed prior to transplant to restore cytokine production to a naive, non-hyper-responsive level and to induce a durable naive, non-hyper-responsive cytokine production level, and favorably decreases the inflammatory to immunosuppressive myeloid cell ratio to the patient for post-transplant acceptance.

In a non-limiting preferred embodiment of the invention, there is provided in any one of methods herein, wherein the nanobiologic composition is administered in a treatment regimen comprising one or more doses to the patient to generate an accumulation of drug in myeloid cells, myeloid progenitor cells, and hematopoietic stem cells in the bone marrow, blood and/or spleen.

### Inhibitors

In a non-limiting preferred embodiment of the invention, there is provided in any one of methods herein, wherein the inhibitor comprises: an inflammasome inhibitor, or an inhibitor of a metabolic pathway or an epigenetic pathway such as a, but not limited to NOD2 receptor inhibitor, an mTOR inhibitor, a ribosomal protein S6 kinase beta-1 (S6K1) inhibitor, an HMG-CoA reductase inhibitor (Statin), a histone H3K27 demethylase inhibitor, a BET bromodomain blockade inhibitor, an inhibitor of histone methyltransferases and acetyltransferases, an inhibitor of DNA methyltransferases and acetyltransferases, a Serine/threonine kinase Akt inhibitor, an Inhibitor of Hypoxia-inducible factor 1-alpha, also known as HIF-1-alpha, and a mixture of one or more thereof.

In a non-limiting preferred embodiment of the invention, there is provided in any one of methods herein, comprising co-treatment with an immunotherapeutic drug as a combination therapy with the nanobiologic composition.

### Nanobiologic Composition

In a non-limiting preferred embodiment of the invention, there is provided a nanobiologic composition for inhibiting trained immunity, comprising:

a nanoscale assembly, having (ii) an inhibitor drug incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell.

In a non-limiting preferred embodiment of the invention, there is provided a nanobiologic composition for inhibiting trained immunity, wherein the nanoscale assembly is a multi-component carrier composition comprising:

a phospholipid or a mixture of phospholipids,
apolipoprotein A-I (apoA-I) or a peptide mimetic of apoA-I, and
a hydrophobic matrix comprised of one or more triglycerides, fatty acid esters, hydrophobic polymers, and sterol esters.

In a non-limiting preferred embodiment of the invention, there is provided a nanobiologic composition for inhibiting trained immunity, wherein the nanoscale assembly is a multi-component carrier composition comprising:

a phospholipid or a mixture of phospholipids,
apolipoprotein A-I (apoA-I) or a peptide mimetic of apoA-I,
a hydrophobic matrix comprised of one or more triglycerides, fatty acid esters, hydrophobic polymers, and sterol esters, and
cholesterol.

In a non-limiting preferred embodiment of the invention, there is provided a nanobiologic composition for inhibiting trained immunity, wherein the inhibitor of a metabolic pathway or an epigenetic pathway comprises: a NOD2 receptor inhibitor, an mTOR inhibitor, a ribosomal protein S6 kinase beta-1 (S6K1) inhibitor, an HMG-CoA reductase inhibitor (Statin), a histone H3K27 demethylase inhibitor, a BET bromodomain blockade inhibitor, an inhibitor of histone methyltransferases and acetyltransferases, an inhibitor of DNA methyltransferases and acetyltransferases, an inflammasome inhibitor, a Serine/threonine kinase Akt inhibitor, an Inhibitor of Hypoxia-inducible factor 1-alpha, also known as HIF-1-alpha, and a mixture of one or more thereof.

### Process for Manufacturing

In a non-limiting preferred embodiment of the invention, there is provided a process for manufacturing a nanobiologic composition for inhibiting trained immunity, comprising the step of:

incorporating an inhibitor drug into a nanoscale assembly;
wherein the nanoscale assembly is a multi-component carrier composition comprising: (a) a phospholipid or a mixture of phospholipids, and
(b) apolipoprotein A-I (apoA-I) or a peptide mimetic of apoA-I,
wherein said nanobiologic, in an aqueous environment, self-assembles into a nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell.

In a non-limiting preferred embodiment of the invention, there is provided a process for manufacturing a nanobiologic composition for inhibiting trained immunity, wherein the nanoscale assembly is a multi-component carrier composition comprising:

a phospholipid or a mixture of phospholipids,
apolipoprotein A-I (apoA-I) or a peptide mimetic of apoA-I, and
a hydrophobic matrix comprised of one or more triglycerides, fatty acid esters, hydrophobic polymers, and sterol esters.

In a non-limiting preferred embodiment of the invention, there is provided a process for manufacturing a nanobiologic composition for inhibiting trained immunity, wherein the nanoscale assembly is a multi-component carrier composition comprising:

a phospholipid or a mixture of phospholipids,
apolipoprotein A-I (apoA-I) or a peptide mimetic of apoA-I,
a hydrophobic matrix comprised of one or more triglycerides, fatty acid esters, hydrophobic polymers, and sterol esters, and
cholesterol.

In a non-limiting preferred embodiment of the invention, there is provided a process for manufacturing, wherein the assembly is combined using microfluidics, high pressure homogenization scale-up microfluidizer technology, sonication, organic-to-aqueous infusion, or lipid film hydration.

### Radiolabelled Nanobiologic and Method of Use

In a non-limiting preferred embodiment of the invention, there is provided a nanobiologic composition for imaging accumulation in bone marrow, blood and spleen, comprising: a nanoscale assembly, having (ii) an inhibitor drug incorporated in the nanoscale assembly, and (iii) a positron emission tomography (PET) imaging radioisotope incorporated in the nanoscale assembly,

wherein the nanoscale assembly is a multi-component carrier composition comprising: (a) a phospholipid or a mixture of phospholipids, and
(b) apolipoprotein A-I (apoA-I) or a peptide mimetic of apoA-I,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹²⁴I, ⁶⁴Cu, ¹⁸F, and ⁸⁶Y, and
wherein the PET imaging radioisotope is complexed to the nanobiologic using a suitable chelating agent to form a stable nanobiologic-radioisotope chelate.

In a further non-limiting preferred embodiment of the invention, there is provided a nanobiologic composition for imaging accumulation in bone marrow, blood and spleen, comprising:

a nanoscale assembly, having (ii) an inhibitor drug incorporated in the nanoscale assembly, and (iii) a positron emission tomography (PET) imaging radioisotope incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I, and
(c) a hydrophobic matrix comprised of one or more triglycerides, fatty acid esters, hydrophobic polymers, and sterol esters,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹241, ⁶⁴Cu, ¹⁸F, and ⁸⁶Y, and
wherein the PET imaging radioisotope is complexed to the nanobiologic using a suitable chelating agent to form a stable nanobiologic-radioisotope chelate.

In a further non-limiting preferred embodiment of the invention, there is provided a nanobiologic composition for imaging accumulation in bone marrow, blood and spleen, comprising:

a nanoscale assembly, having (ii) an inhibitor drug incorporated in the nanoscale assembly, and (iii) a positron emission tomography (PET) imaging radioisotope incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I,
(c) a hydrophobic matrix comprised of one or more triglycerides, fatty acid esters, hydrophobic polymers, and sterol esters, and
(d) cholesterol,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹²⁴I, ⁶⁴Cu, ¹⁸F, and ⁸⁶Y, and
wherein the PET imaging radioisotope is complexed to the nanobiologic using a suitable chelating agent to form a stable nanobiologic-radioisotope chelate.

In a non-limiting preferred embodiment of the invention, there is provided a method of positron emission tomography (PET) imaging the accumulation of a nanobiologic within bone marrow, blood, and/or spleen, of a patient affected by trained immunity, comprising: administering to said patient a nanobiologic composition for imaging accumulation in bone marrow, blood and spleen, comprising:

a nanoscale assembly, having (ii) an inhibitor drug incorporated in the nanoscale assembly, and (iii) a positron emission tomography (PET) imaging radioisotope incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹²⁴I, ⁶⁴Cu, ¹⁸F, and ⁸⁶Y, and
wherein the PET imaging radioisotope is complexed to the nanobiologic using a suitable chelating agent to form a stable nanobiologic-radioisotope chelate, and
(2) performing PET imaging of the patient to visualize biodistribution of the stable nanobiologic-radioisotope chelate within the bone marrow, blood, and/or spleen of the patient's body.

In a further non-limiting preferred embodiment of the invention, there is provided a method of positron emission tomography (PET) imaging the accumulation of a nanobiologic within bone marrow, blood, and/or spleen, of a patient affected by trained immunity, comprising: administering to said patient a nanobiologic composition for imaging accumulation in bone marrow, blood and spleen, comprising:

a nanoscale assembly, having (ii) an inhibitor drug incorporated in the nanoscale assembly, and (iii) a positron emission tomography (PET) imaging radioisotope incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I, and
(c) a hydrophobic matrix comprised of one or more triglycerides, fatty acid esters, hydrophobic polymers, and sterol esters,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹²⁴I, ⁶⁴Cu, ¹⁸F, and ⁸⁶Y, and
wherein the PET imaging radioisotope is complexed to the nanobiologic using a suitable chelating agent to form a stable nanobiologic-radioisotope chelate, and
(2) performing PET imaging of the patient to visualize biodistribution of the stable nanobiologic-radioisotope chelate within the bone marrow, blood, and/or spleen of the patient's body.

In a non-limiting preferred embodiment of the invention, there is provided a method of positron emission tomography (PET) imaging the accumulation of a nanobiologic within bone marrow, blood, and/or spleen, of a patient affected by trained immunity, comprising: administering to said patient a nanobiologic composition for imaging accumulation in bone marrow, blood and spleen, comprising:

a nanoscale assembly, having (ii) an inhibitor drug incorporated in the nanoscale assembly, and (iii) a positron emission tomography (PET) imaging radioisotope incorporated in the nanoscale assembly,
wherein the nanoscale assembly is a multi-component carrier composition comprising: (a) a phospholipid or a mixture of phospholipids, and
(b) apolipoprotein A-I (apoA-I) or a peptide mimetic of apoA-I,
(c) a hydrophobic matrix comprised of one or more triglycerides, fatty acid esters, hydrophobic polymers, and sterol esters, and
(d) cholesterol,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell, and
wherein the PET imaging radioisotope is selected from ⁸⁹Zr, ¹²⁴I, ⁶⁴Cu, ¹⁸F, and ⁸⁶Y, and
wherein the PET imaging radioisotope is complexed to the nanobiologic using a suitable chelating agent to form a stable nanobiologic-radioisotope chelate, and
(2) performing PET imaging of the patient to visualize biodistribution of the stable nanobiologic-radioisotope chelate within the bone marrow, blood, and/or spleen of the patient's body.

### BRIEF DESCRIPTION OF THE OF DRAWINGS

### Transplantation

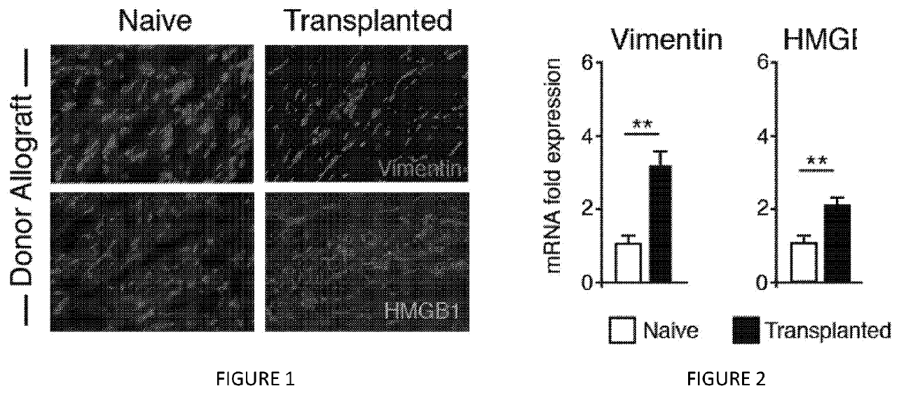

FIG. 1 is an immunostaining panel of four images of vimentin and HMGB1 expression in donor and non-transplanted hearts (n=3/mice per group of three independent experiments, t-test; **P<0.01) and shows vimentin and HMGB1 are upregulated following organ transplantation and promote training of graft infiltrating macrophages.

FIG. 2 is a graph of mRNA fold expression in real-time PCR of vimentin and HMGB1 expression in donor and non-transplanted hearts (n=3/mice per group of three independent experiments, t-test; **P<0.01) and shows vimentin and HMGB1 are upregulated following organ transplantation and promote training of graft infiltrating macrophages.

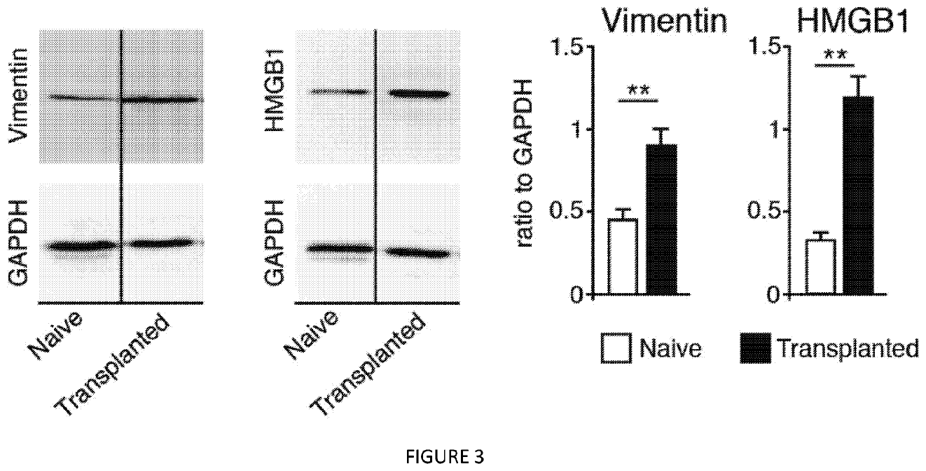

FIG. 3 is a panel of four images of western blot analysis next to a two-panel bar graph of vimentin and HMGB1 expression in donor and non-transplanted hearts (n=3/mice per group of three independent experiments, t-test; **P<0.01) and shows vimentin and HMGB1 are upregulated following organ transplantation and promote training of graft infiltrating macrophages.

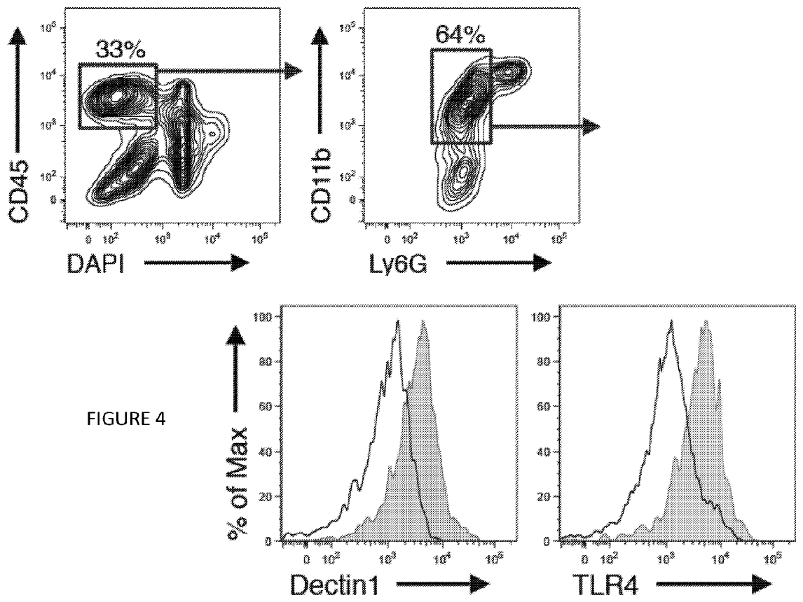

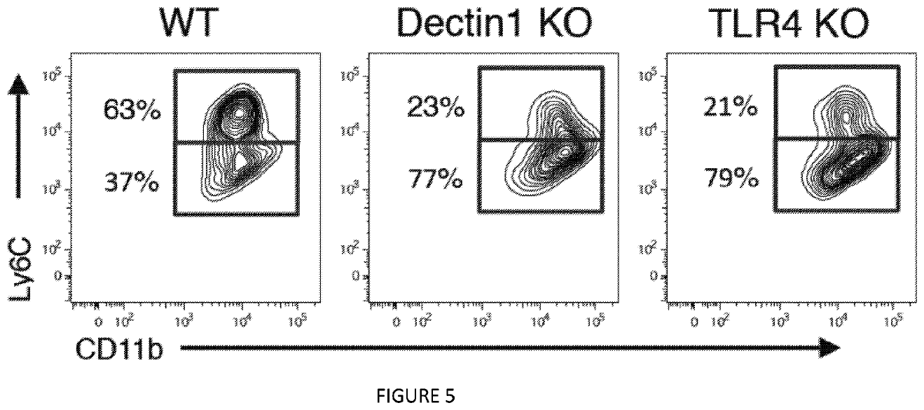

FIG. 4 is a four-panel illustration of flow cytometry analysis and shows dectin-1 and TLR4 expression in graft infiltrating macrophages (n=3 mice/group of two independent experiments).

FIG. 5 is a three-panel illustration of flow cytometry analysis and shows Ly-6C expression in graft infiltrating macrophages from WT, dectin1 KO and TLR4 KO untreated recipient mice (n=3 mice/group of two independent experiments).

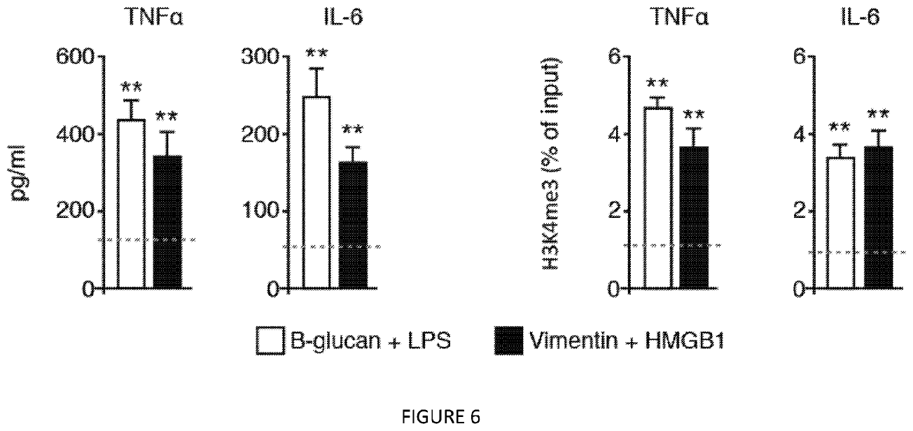

FIG. 6 is a four-panel bar graph illustration and shows Inflammatory cytokine production and chromatin immunoprecipitation of mouse monocytes trained with vimentin and HMGB, and β-glucan and LPS (n=3 independent experiments, one-way ANOVA, **P<0.01; dashed line displays control non-trained conditions).

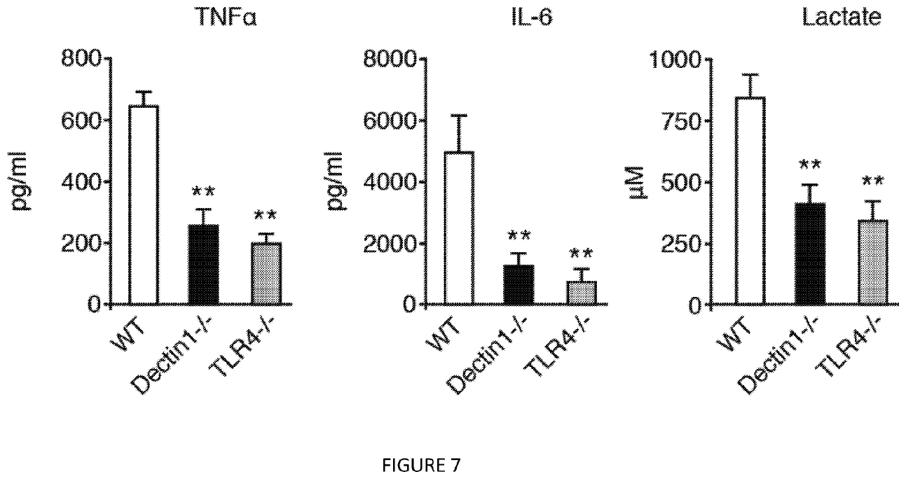

FIG. 7 is a three-panel bar graph illustration and shows cytokine and lactate production of graft-infiltrating macrophages (n=4 mice/group of 2 independent experiments, one-way ANOVA, **P<0.01).

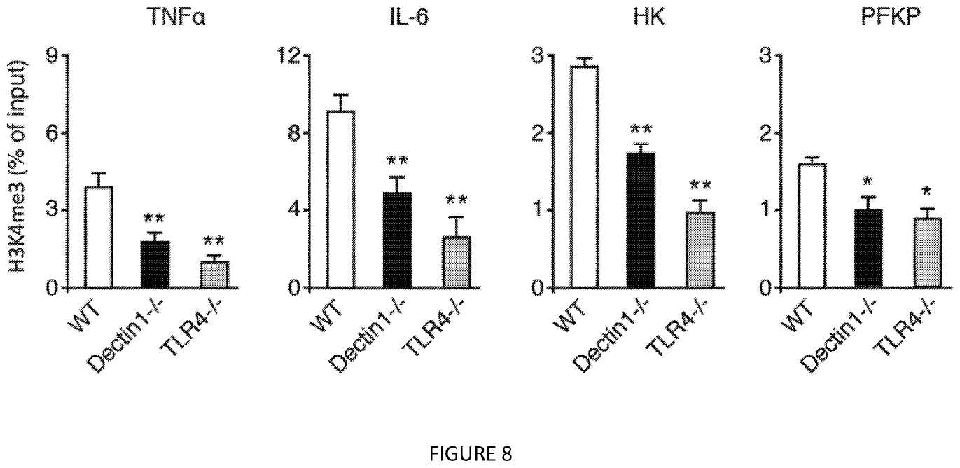

FIG. 8 is a four-panel bar graph illustration and shows chromatin immunoprecipitation of graft-infiltrating macrophages (n=4 mice/group of 2 independent experiments, one-way ANOVA, *P<0.05; **P<0.01).

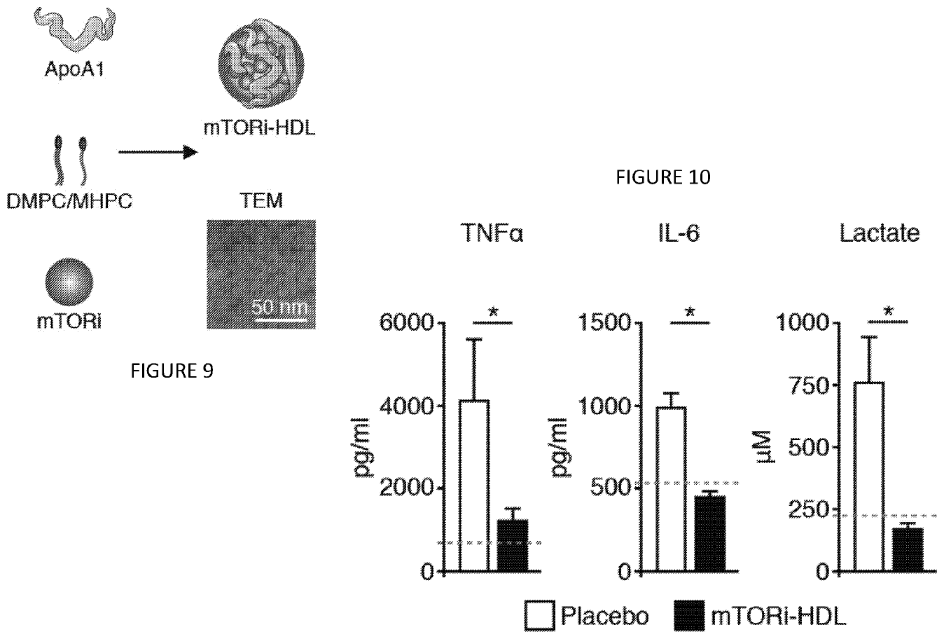

FIG. 9 is a graphic illustration of components and assembly of one non-limiting example of an inhibitor-HDL complex, apolipoprotein A1 (apoA1, also named as apolipoprotein A-I or apoA-I) plus a mixture of double-chain and single-chain phosphocholine compounds (DMPC/MHPC) plus a mammalian Target of Rapamycin inhibitor (mTORi) to form an Inhibitor-HDL complex as mTORi-HDL, with a 50 nm scale image of transmission electron microscopy (TEM) of mTORi-HDL nanobiologics. FIG. 9 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

FIG. 10 is a three-panel graph and shows cytokine and lactate production of human macrophages trained in vitro (n=3 independent experiments, t-test, *P<0.05; dashed line displays control non-β-glucan trained condition). FIG. 10 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

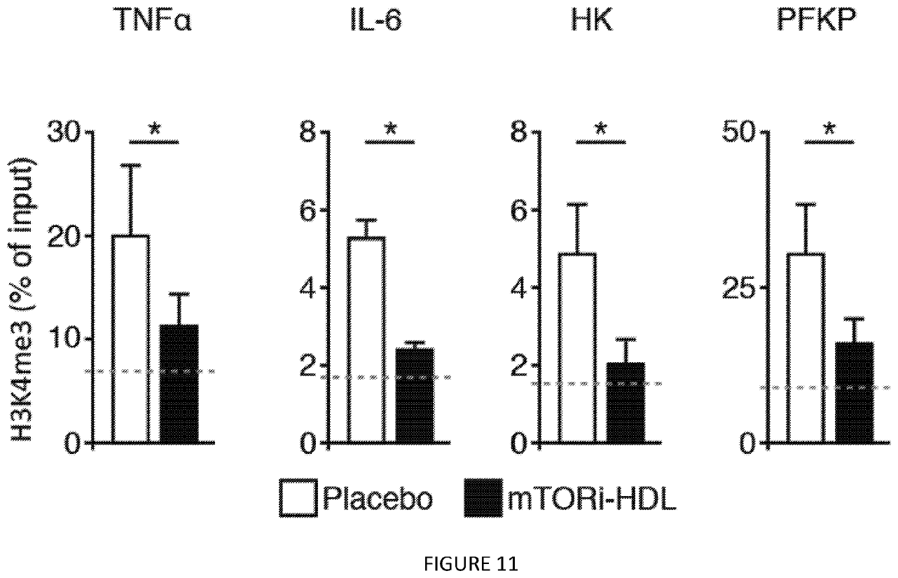

FIG. 11 is a four-panel graph and shows chromatin immunoprecipitation of human macrophages trained in vitro (n=3 independent experiments, t-test, *P<0.05; dashed line displays control non-β-glucan trained condition). FIG. 11 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

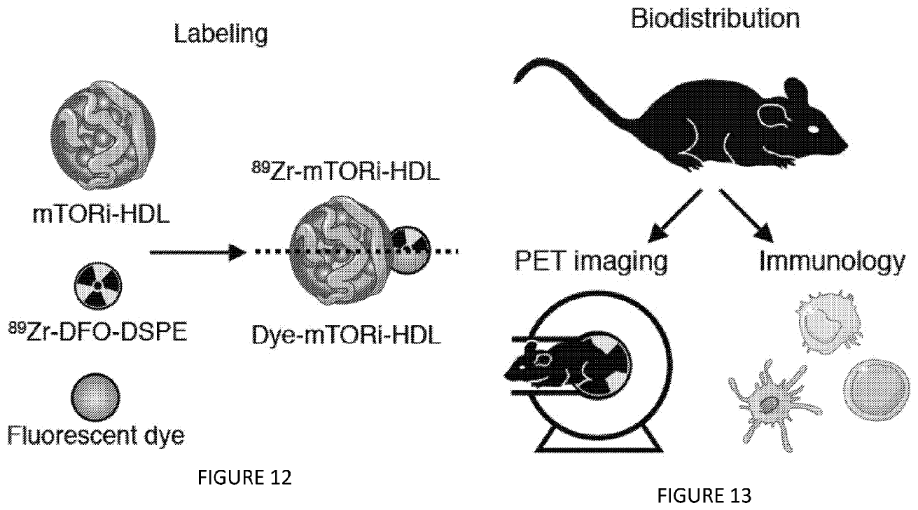

FIG. 12 is a graphic illustration of labelling components and assembly of one non-limiting example of a labelled Inhibitor-HDL complex. Labeling of mTORi-HDL with either the radioisotope ⁸⁹Zr or the fluorescent dyes DiO or DiR. FIG. 12 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

FIG. 13 is a graphic illustration of micro-PET/CT and cellular specificity of mTORi-HDL nanobiologics. FIG. 13 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

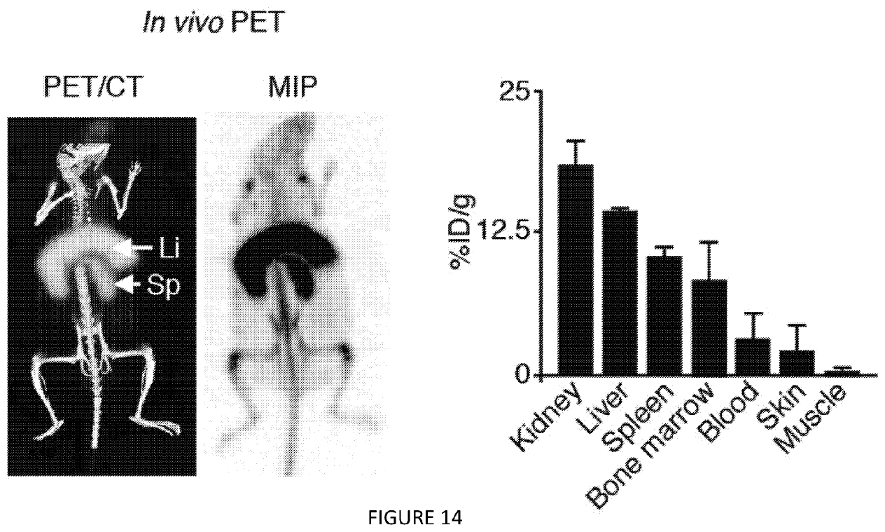

FIG. 14 is a representative micro-PET/CT 3D fusion image and PET maximum intensity projection graph (MIP) and graph of the results (mean±SEM, n=3). FIG. 14 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

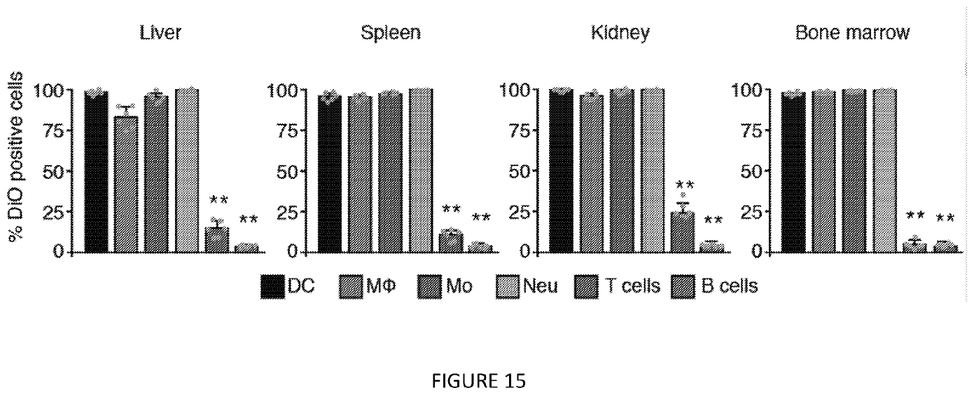

FIG. 15 is a four-panel graph illustration of uptake of fluorescently labeled DiO mTORi-HDL by myeloid and lymphoid cells (n=5 mice/group, one-way ANOVA, **P<0.01). FIG. 15 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

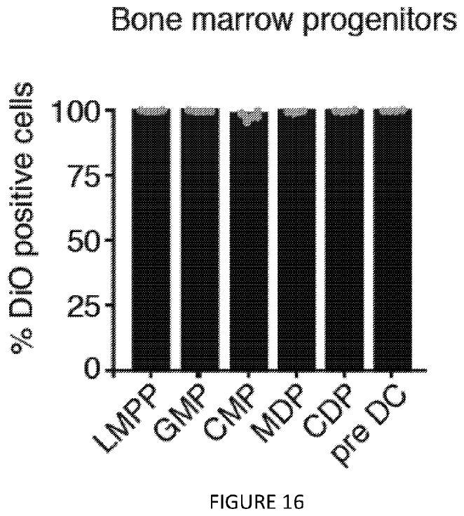

FIG. 16 is a single-panel graph of uptake of fluorescently labeled DiG mTORi-HDL by bone marrow progenitors (mean±SEM, n=5). FIG. 16 shows in one aspect that mTORi-HDL nanoimmunotherapy prevents trained immunity to the level of naive cells, and avidity to myeloid cells in blood, and stem cell and progenitors in bone marrow and in spleen in vitro and distributes systemically in vivo.

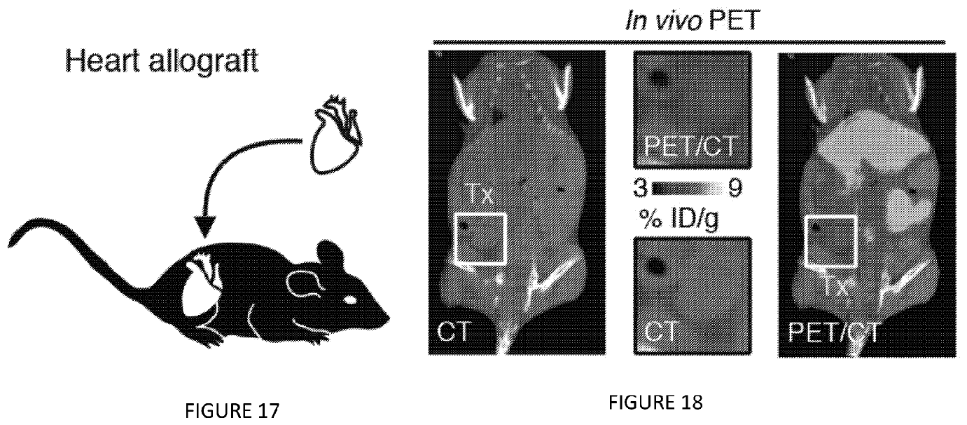

FIG. 17 is a graphic illustration of BALB/c donor hearts (H2d) transplanted into fully allogeneic C57BL/6 recipients (H2b). FIG. 17 shows in one aspect that mTORi-HDL nanoimmunotherapy targets myeloid cells in the allograft and prevents trained immunity.

FIG. 18 is a series of panel images of micro-PET/CT 3D fusion image 24 hours after intravenous administration of ⁸⁹Zr-mTORi-HDL (n=3 mice/group of 2 independent experiments). FIG. 18 shows in one aspect that mTORi-HDL nanoimmunotherapy targets myeloid cells in the allograft and prevents trained immunity.

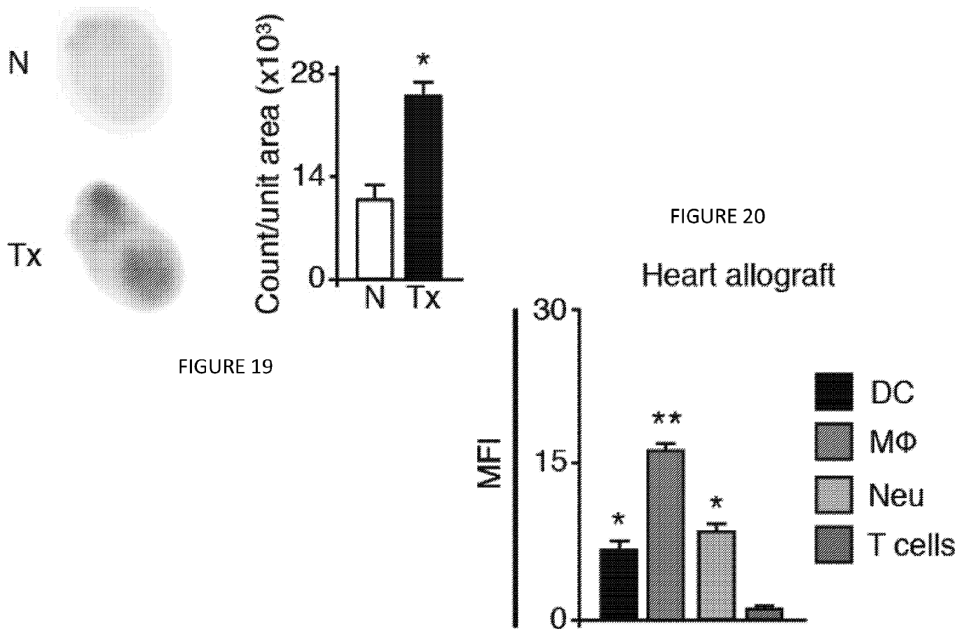

FIG. 19 is a pair of images and a graph of ex vivo autoradiography in native (N) and transplanted hearts (Tx) at 24 hours after intravenous ⁸⁹Zr-mTORi-HDL (n=3 mice/group of 2 independent experiments, t-test, *P<0.05). FIG. 19 shows in one aspect that mTORi-HDL nanoimmunotherapy targets myeloid cells in the allograft and prevents trained immunity.

FIG. 20 is a bar graph of uptake of fluorescently labeled DiO mTORi-HDL by myeloid and lymphoid cells in the allograft (n=4 mice/group of 3 independent experiments; one-way ANOVA, *P<0.05; **P<0.01). FIG. 20 shows in one aspect that mTORi-HDL nanoimmunotherapy targets myeloid cells in the allograft and prevents trained immunity.

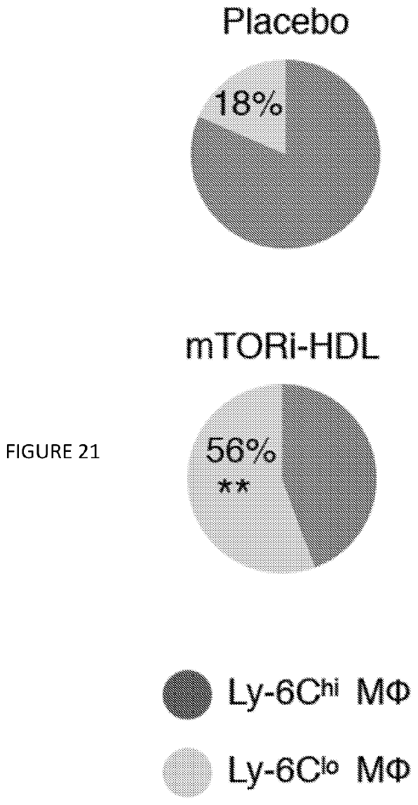

FIG. 21 is a pair of pie charts of Ly-6Chi/Ly-6Clo MΦ ratio in the allograft from either placebo or mTORi-HDL-treated recipients at day 6 post-transplantation (n=4 mice/group of 3 independent experiments; one-way ANOVA, *P 0.05; **P<0.01). FIG. 21 shows in one aspect that mTORi-HDL nanoimmunotherapy targets myeloid cells in the allograft and prevents trained immunity.

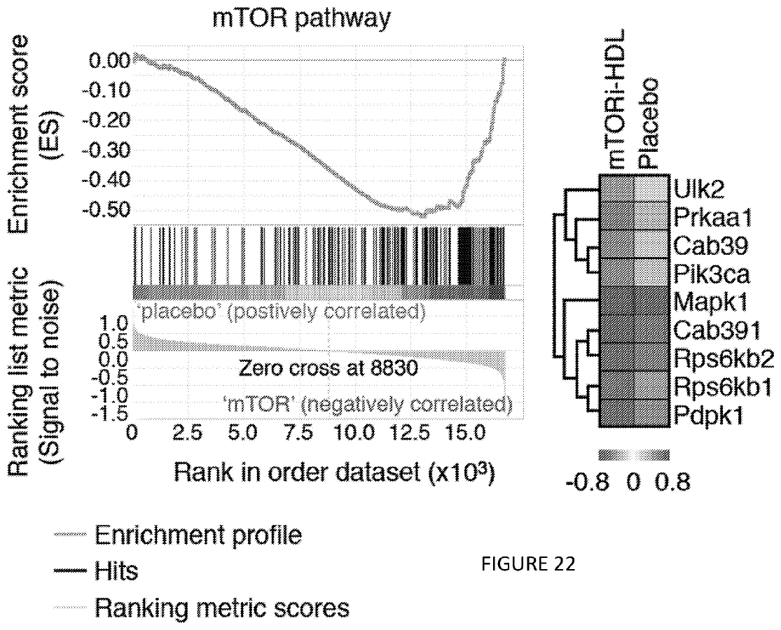

FIG. 22 is one of a pair of graphs of GSEA gene array analysis for the mTOR and glycolysis pathways in intra-graft MΦ from placebo or mTORi-HDL-treated recipients (n=3 mice/group). FIG. 22 shows in one aspect that mTORi-HDL nanoimmunotherapy targets myeloid cells in the allograft and prevents trained immunity.

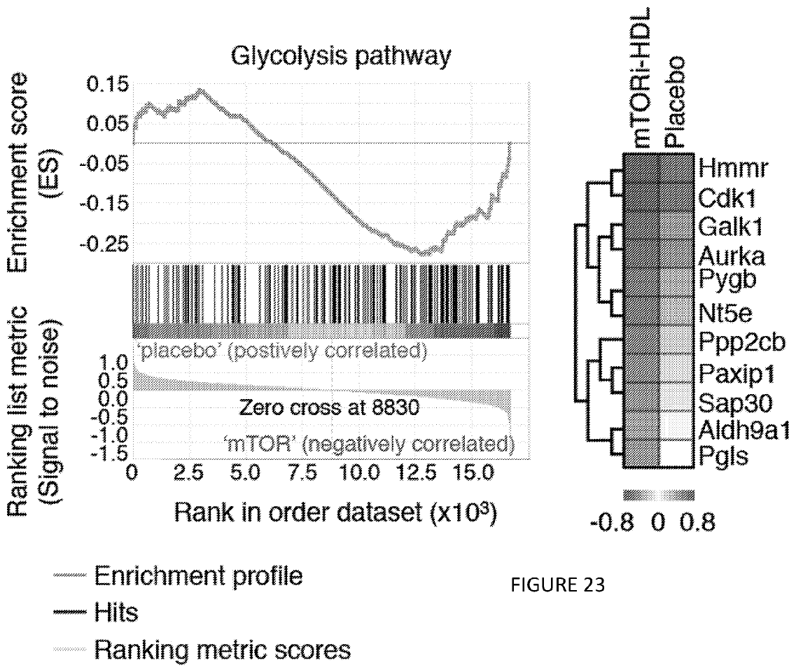

FIG. 23 is the second of a pair of graphs of GSEA gene array analysis for the mTOR and glycolysis pathways in intra-graft MΦ from placebo or mTORi-HDL-treated recipients (n=3 mice/group). FIG. 23 shows in one aspect that mTORi-HDL nanoimmunotherapy targets myeloid cells in the allograft and prevents trained immunity.

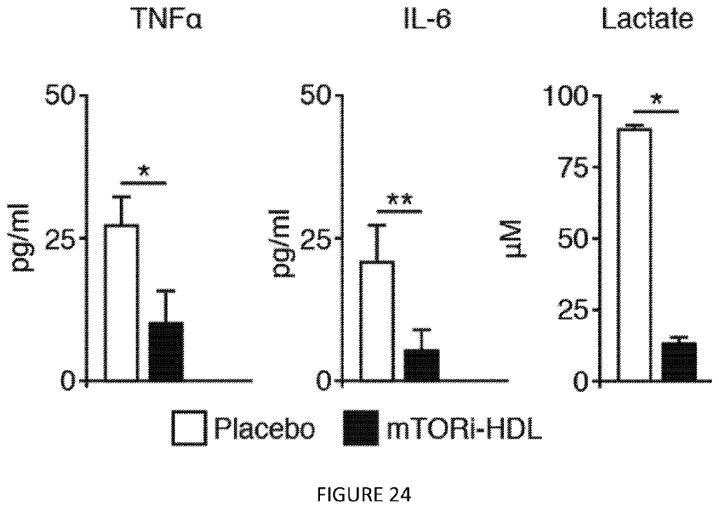

FIG. 24 is a three-panel illustration of bar graphs of cytokine and lactate production of graft-infiltrating macrophages from either placebo or mTORi-HDL-treated recipients (n=4 mice/group of 3 independent experiments, t-test, *P<0.05; **P<0.01). FIG. 24 shows in one aspect that mTORi-HDL nanoimmunotherapy targets myeloid cells in the allograft and prevents trained immunity.

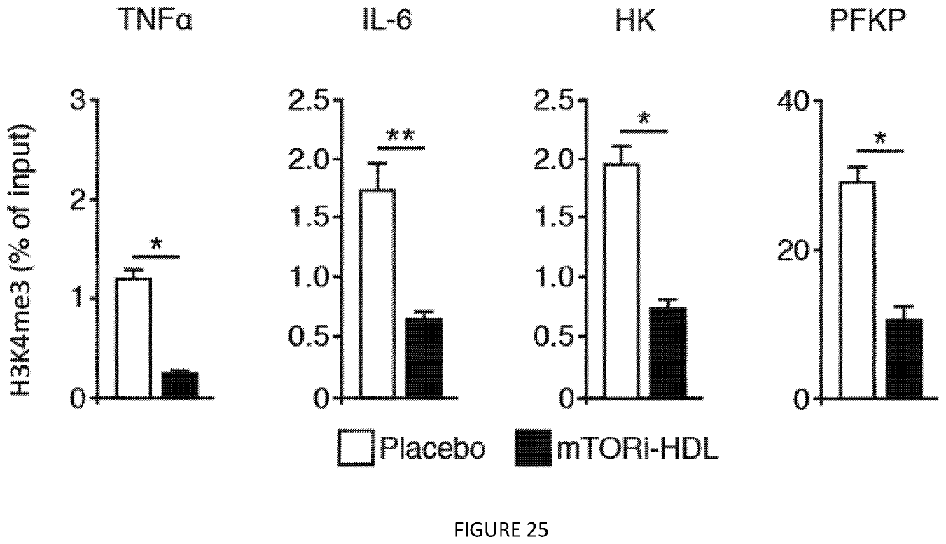

FIG. 25 is a four-panel illustration of bar graphs of chromatin immunoprecipitation of graft-infiltrating macrophages from either placebo or mTORi-HDL-treated recipients (n=4 mice/group of 3 independent experiments, t-test, *P<0.05; **P<0.01). FIG. 25 shows in one aspect that mTORi-HDL nanoimmunotherapy targets myeloid cells in the allograft and prevents trained immunity.

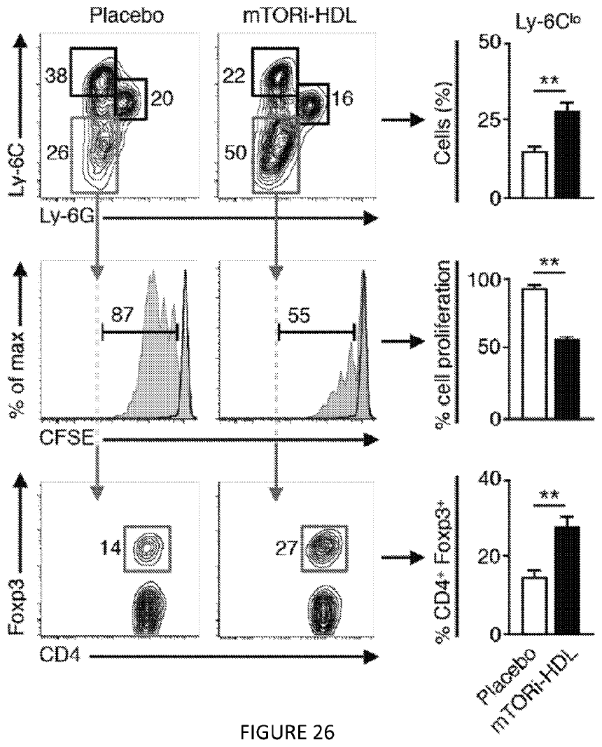

FIG. 26 is a nine-panel graph illustration of functional characterization of graft-infiltrating M(D from placebo and mTORi-HDL-treated recipients using CD8 T cell suppressive and CD4 Treg expansion assays (n=4 mice/group of 3 independent experiments, t-test, **P≤0.01). FIG. 26 shows in one aspect that a combination of mTORi-HDL trained immunity nanoimmunotherapy, and CD40 activation of T cells (not Trained Immunity), as a synergistic therapy, promotes organ transplant acceptance.

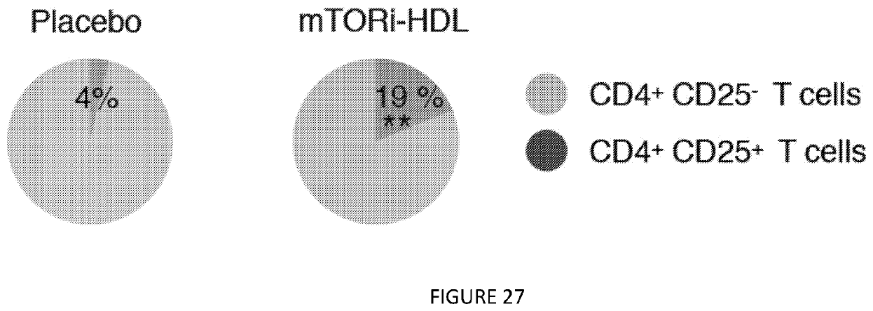

FIG. 27 is a pair of pie charts of a percentage of graft-infiltrating CD4+CD25+ Treg cells from placebo and mTORi-HDL-treated recipients (n=4 mice/group of 3 independent experiments, t-test, **P<0.01). FIG. 27 shows in one aspect that a combination of mTORi-HDL trained immunity nanoimmunotherapy, and CD40 activation of T cells (not Trained Immunity), as a synergistic therapy, promotes organ transplant acceptance.

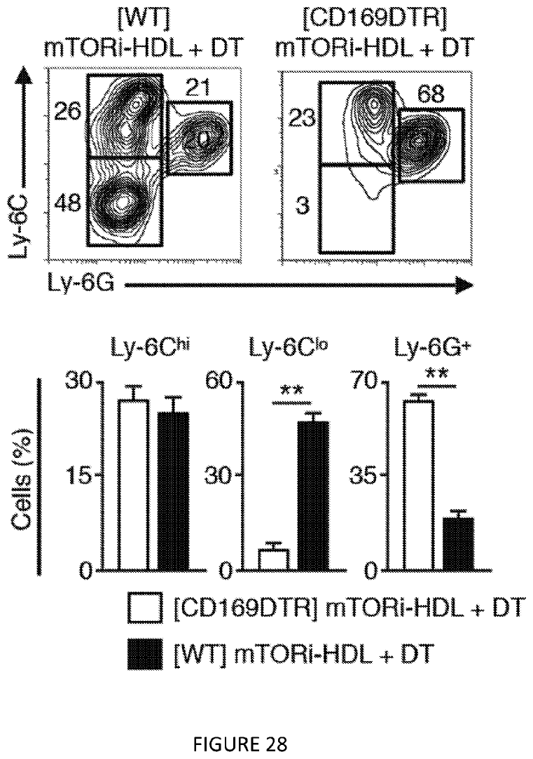

FIG. 28 is a five-panel graph illustration of depletion of CD169+ graft-infiltrating Mreg in placebo and mTORi-HDL-treated recipients (n=5 mice/group of 3 independent experiments, t-test, **P<0.01). FIG. 28 shows in one aspect that a combination of mTORi-HDL trained immunity nanoimmunotherapy, and CD40 activation of T cells (not Trained Immunity), as a synergistic therapy, promotes organ transplant acceptance.

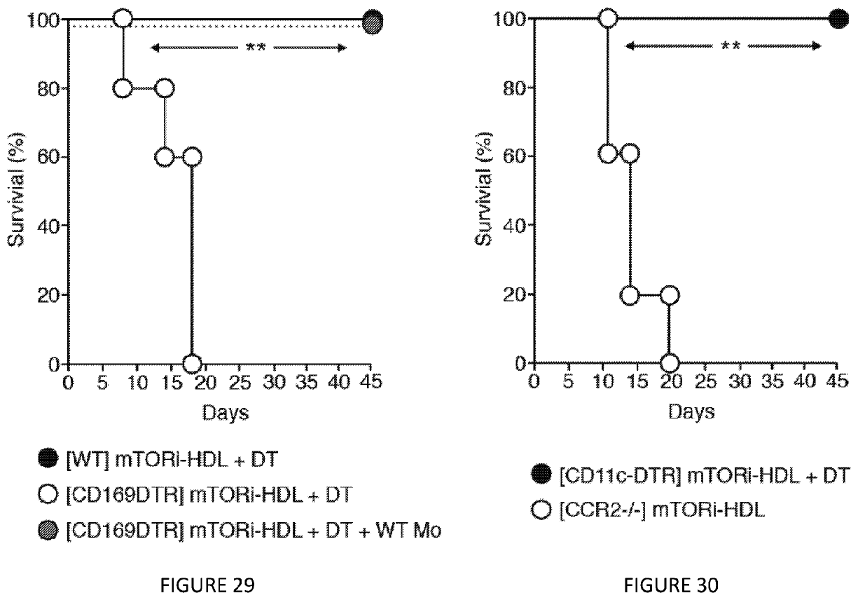

FIG. 29 is a line graph of graft survival following depletion CD169+ graft-infiltrating Mreg (n=5 mice/group; Kaplan-Meier **P≤0.01). FIG. 29 shows in one aspect that a combination of mTORi-HDL trained immunity nanoimmunotherapy, and CD40 activation of T cells (not Trained Immunity), as a synergistic therapy, promotes organ transplant acceptance.

FIG. 30 is a line graph of graft survival following depletion of CD11c+ cells and in CCR2 deficient recipient mice (n=5 mice/group, Kaplan-Meier, **P<0.01). FIG. 30 shows in one aspect that a combination of mTORi-HDL trained immunity nanoimmunotherapy, and CD40 activation of T cells (not Trained Immunity), as a synergistic therapy, promotes organ transplant acceptance.

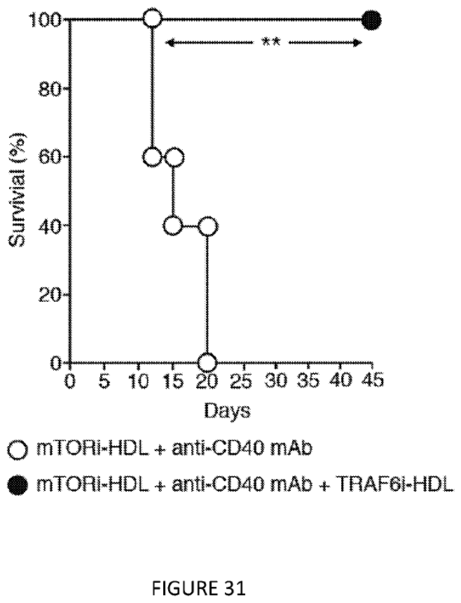

FIG. 31 is a line graph of graft survival of mTORi-HDL-treated recipients receiving agonistic stimulatory CD40 mAb in vivo with or without TRAF6i-HDL nanoimmunotherapy (n=5 mice/group, Kaplan-Meier, **P<0.01). FIG. 31 shows in one aspect that a combination of mTORi-HDL trained immunity nanoimmunotherapy, and CD40 activation of T cells (not Trained Immunity), as a synergistic therapy, promotes organ transplant acceptance.

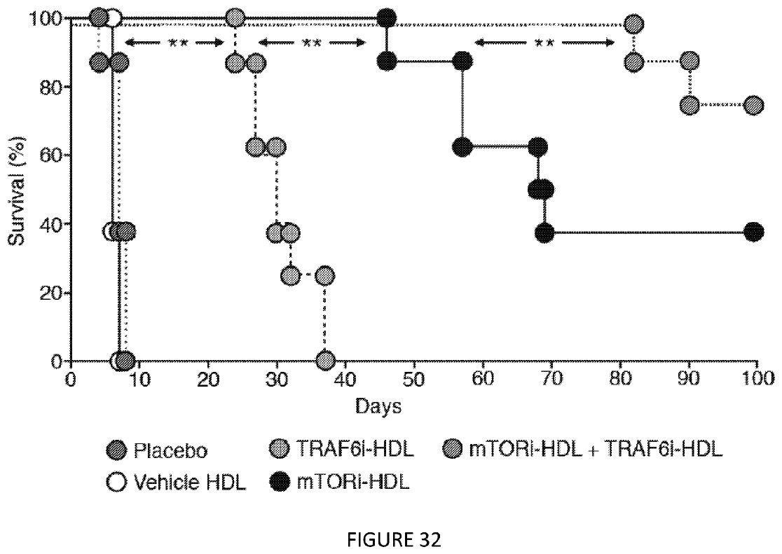

FIG. 32 is a line graph of graft survival of placebo, vehicle HDL, mTORi-HDL, TRAF6i-HDL and mTORi-HDL/TRAF6i-HDL treated recipients (n=7-8 mice/group, Kaplan-Meier, **P<0.01). FIG. 32 shows in one aspect that a combination of mTORi-HDL trained immunity nanoimmunotherapy, and CD40 activation of T cells (not Trained Immunity), as a synergistic therapy, promotes organ transplant acceptance.

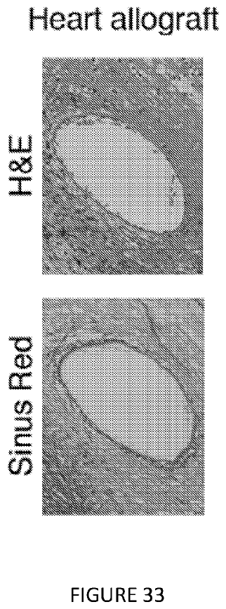

FIG. 33 is a two-panel image of immunohistochemistry of heart allografts from mTORi-HDL/TRAF6i-HDL-treated recipients on day 100 after transplantation (n=5 mice/group; magnification ×200). FIG. 33 shows in one aspect that a combination of mTORi-HDL trained immunity nanoimmunotherapy, and CD40 activation of T cells (not Trained Immunity), as a synergistic therapy, promotes organ transplant acceptance.

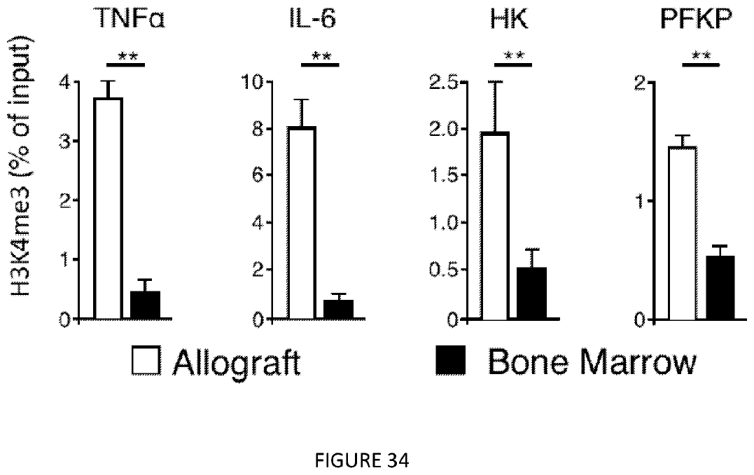

FIG. 34 is a four-panel series of bar graphs of chromatin immunoprecipitation assay (ChIP) of graft-infiltrating and bone marrow monocytes from untreated rejecting recipients at day 6 post-transplantation. ChIP was performed to evaluate histone H3K4 trimethylation. Abundance of four trained immunity-related genes was examined by qPCR (n=3, Wilcoxon signed rank test, **P<0.01. Results from 1 experiment). FIG. 34 shows in one aspect the development and in vivo distribution of mTORi-HDL.

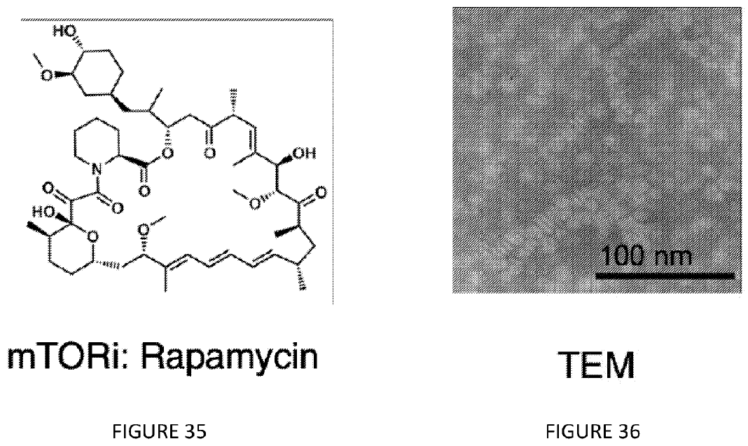

FIG. 35 is an illustration of the chemical structure of the mTOR inhibitor (mTORi) rapamycin.

FIG. 36 is an image of transmission electron micrograph showing the discoidal morphology of mTORi-HDL nanobiologic.

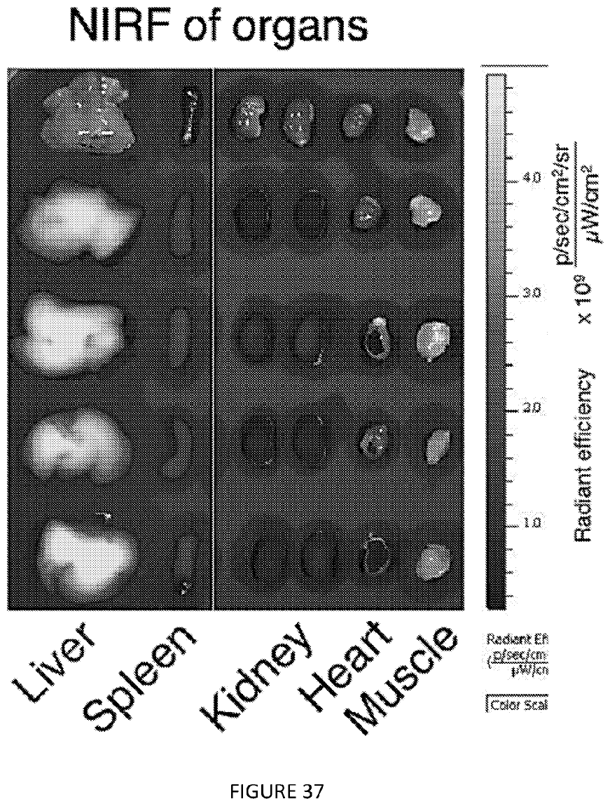

FIG. 37 is a graphic bar-chart illustration of images of mTORi-HDL's biodistribution in C57/B16 wild type mice. Representative near infrared fluorescence images (NIRF) of organs injected with either PBS control (first row of organs) or DiR-labeled mTORi-HDL showing accumulation in liver, spleen, lung, kidney, heart and muscle. FIG. 37 shows in one aspect the development and in vivo distribution of mTORi-HDL.

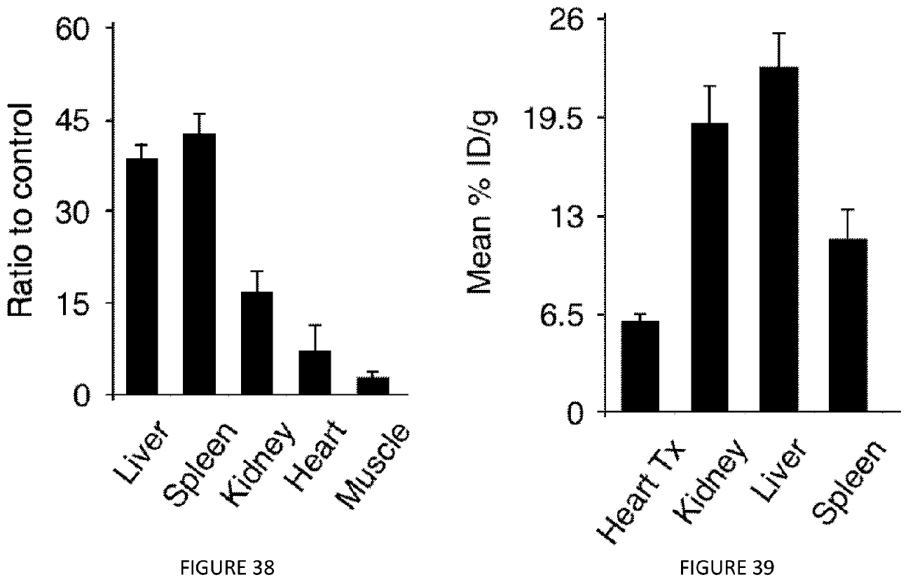

FIG. 38 is a bar chart where bars represent the control to mTORi-HDL-DiR accumulation ratio in each organ, calculated by dividing the total signal of each organ in the control and mTORi-HDL-DiR groups (n=4 mice/group. Results from 3 experiments).

FIG. 38 shows in one aspect the development and in vivo distribution of mTORi-HDL.

FIG. 39 is a bar chart where PET-quantified uptake values according to the mean % ID/g in transplanted heart, kidney, liver and spleen (n=3 mice. Results from 3 experiments).

FIG. 39 shows in one aspect the development and in vivo distribution of mTORi-HDL.

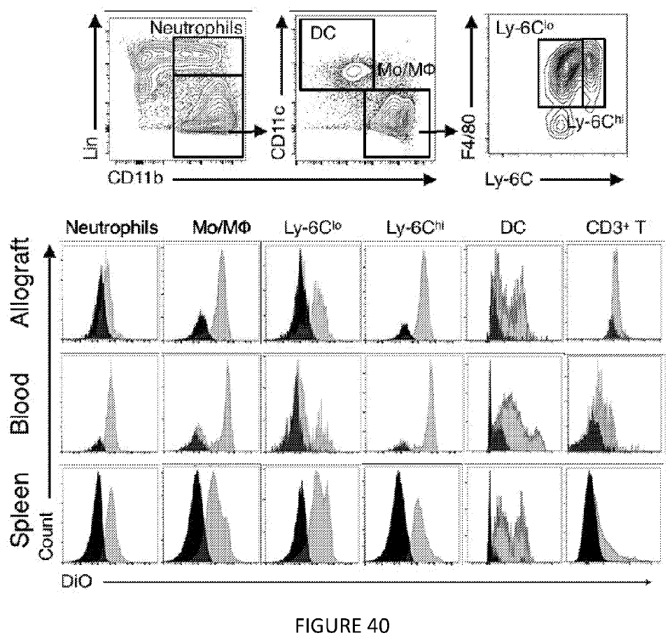

FIG. 40 is a twenty-one panel illustration of flow cytometry gating strategy to distinguish myeloid cells in blood, spleen and the transplanted heart. Grey histograms show immune cell distribution in the mice injected with DiO-labeled mTORi-HDL compared to control (black histogram). FIG. 40 shows in one aspect the in vivo cellular targeting of mTORi-HDL.

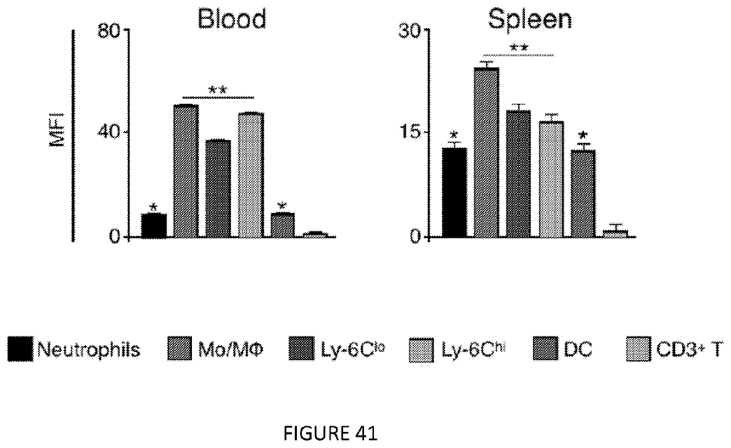

FIG. 41 is a two-panel bar graph illustration of mean fluorescence intensity (MFI) of neutrophils, monocytes/macrophages, Ly-6C lo and Ly-6C hi monocytes/macrophages, dendritic cells and T cells in the blood and spleen (n=4 mice/group, one-way ANOVA, *P<0.05; **P<0.01. Results from 3 experiments). FIG. 41 shows in one aspect the in vivo cellular targeting of mTORi-HDL.

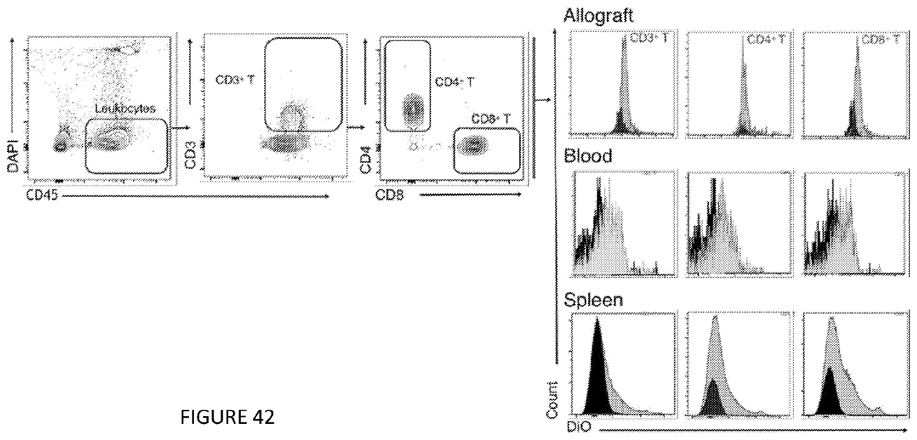

FIG. 42 is a three-panel graphic illustration with a nine-panel graphic illustration of flow cytometry gating strategy to distinguish T cells in blood, spleen and the transplanted heart. Grey histograms (right) show the T cell distribution in mice injected with DiO-labeed mTORi-HDL compared to distribution in control animals (black histogram). FIG. 42 shows in one aspect the In vivo cellular targeting of mTORi-HDL.

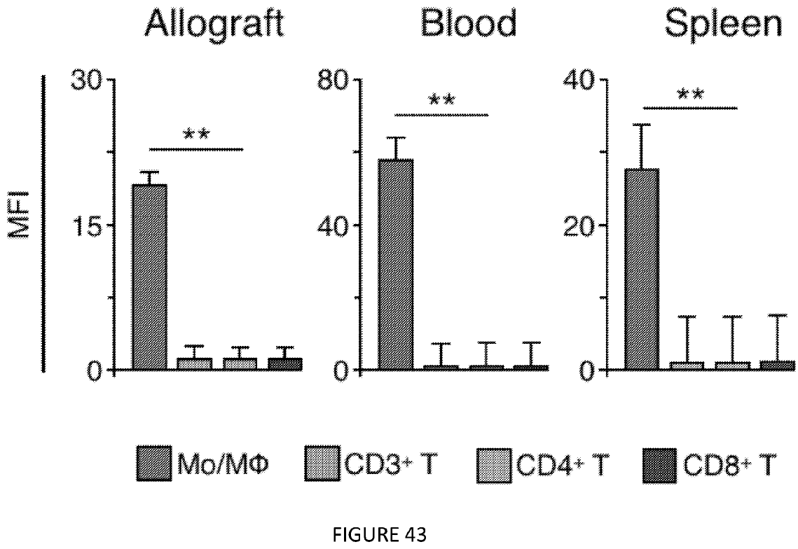

FIG. 43 is a three-panel graphic illustration of mean fluorescence intensity (MFI) of monocytes/macrophages, CD3+T, CD4+T and CD8+ T cells in blood and the transplanted heart (n=4 mice/group, one-way ANOVA, **P<0.01. Results from 3 experiments). FIG. 43 shows in one aspect the in vivo cellular targeting of mTORi-HDL.

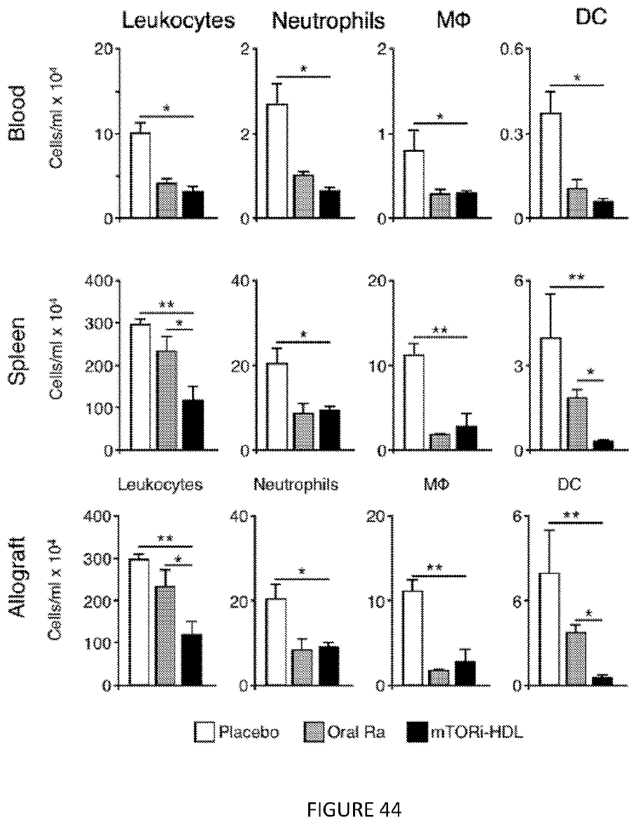

FIG. 44 is a twelve-panel graphic illustration of flow cytometric analysis of cell suspensions retrieved from allograft, blood and spleen of placebo, oral rapamycin (5 mg/kg) and mTORi-HDL-treated (5 mg/kg) allograft recipients at day 6 post transplantation. Total numbers of leukocytes, neutrophils, macrophages (M(D) and dendritic cells (DC) are shown (n=4 mice/group, one-way ANOVA, *P<0.05; **P<0.01. Results from 3 experiments).

FIG. 44 shows in one aspect that mTORi-HDL rebalances the myeloid and Treg compartment in vivo.

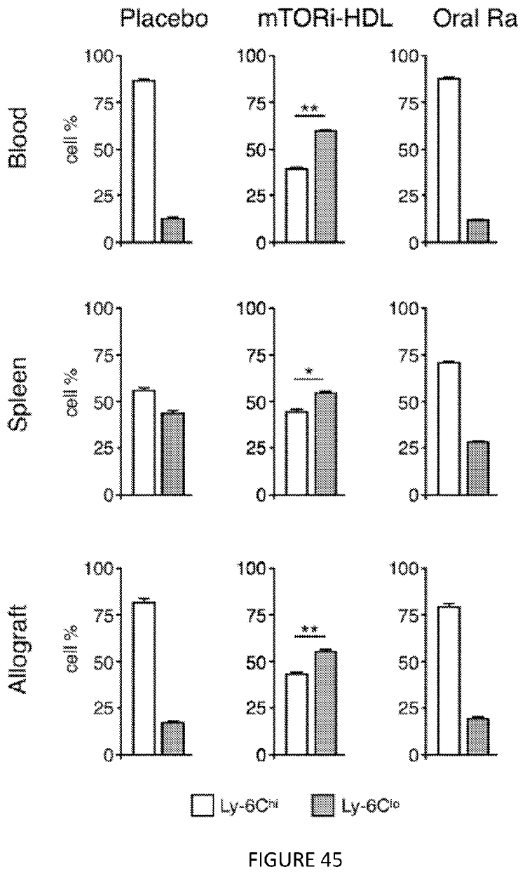

FIG. 45 is a nine-panel graphic illustration of the ratio of Ly-6C to Ly-6C^{lo} monocytes in the blood, spleen and heart allograft from placebo, oral rapamycin (5 mg/kg) and mTORi-HDL-treated (5 mg/kg) allograft recipients (n=4 per group, one-way ANOVA, *P<0.05; **P<0.01. Results from 3 experiments). FIG. 45 shows in one aspect that mTORi-HDL rebalances the myeloid and Treg compartment in vivo.

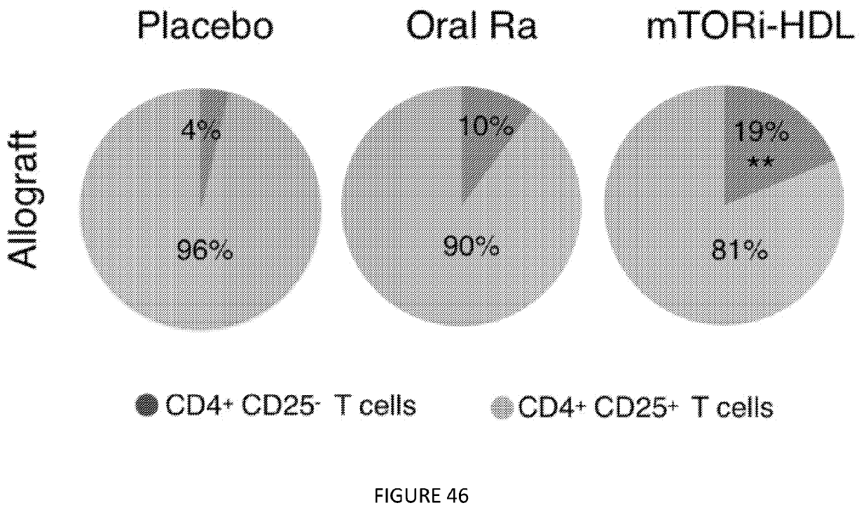

FIG. 46 is a three-panel pie chart illustration of the percentage of graft-infiltrating CD4+CD25+vs. CD4+CD25− T-cells from placebo, oral rapamycin (5 mg/kg) and mTORi-HDL-treated (5 mg/kg) allograft recipients (n=4 mice/group, one-Way ANOVA, **P<0.01. Results from 3 experiments). FIG. 46 shows in one aspect that mTORi-HDL rebalances the myeloid and Treg compartment in vivo.

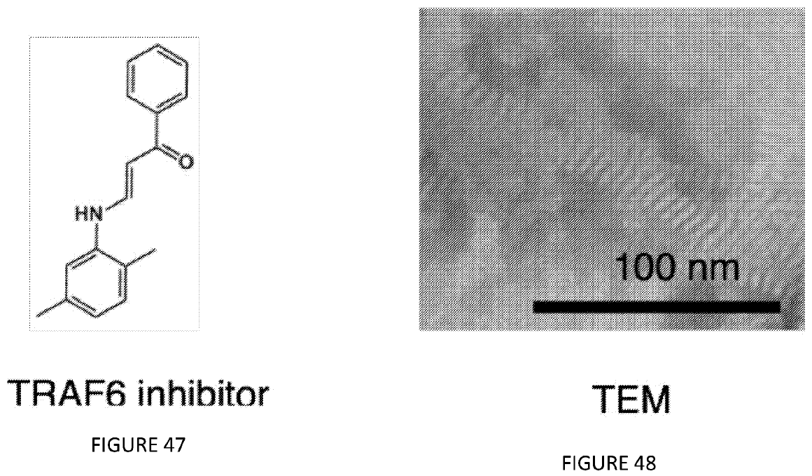

FIG. 47 is an illustration of the chemical structure of a TRAF6 inhibitor, which is the non-trained immunity part of the synergistic combination therapy with a trained immunity nanoimmunotherapeutic.

FIG. 48 is an image of transmission electron micrograph showing the discoidal morphology of TRAF6i-HDL. The nanoparticles had a mean hydrodynamic radius of 19.2±3.1 nm and a drug incorporation efficiency of 84.6±8.6%, as determined by DLS and HPLC, respectively.

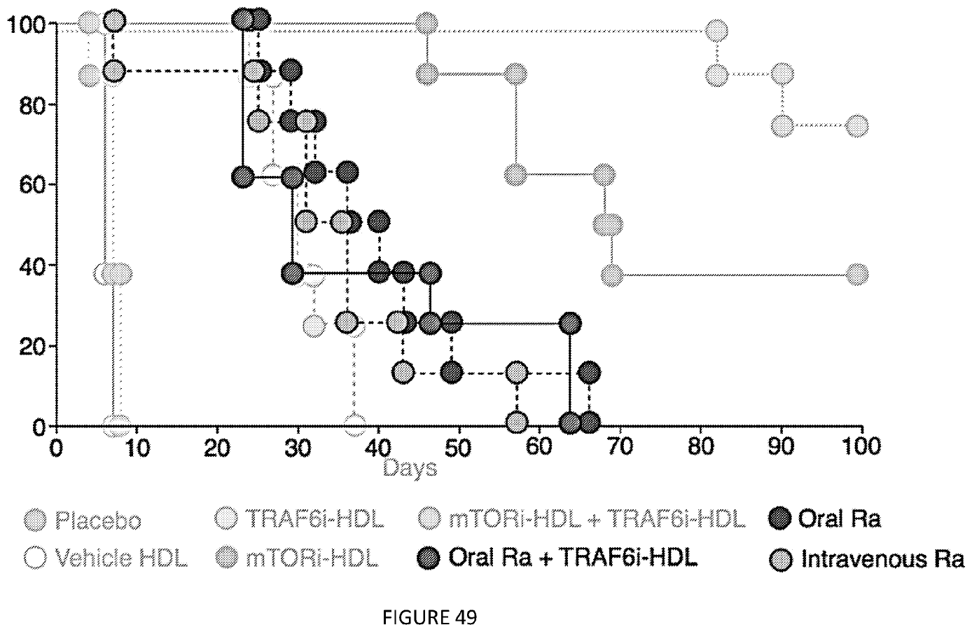

FIG. 49 is a line graph of graft survival curves of oral rapamycin, Intravenous rapamycin and oral rapamycin+ TRAF6i-HDL (n=8 mice in each group). The background shows graft survival curves for placebo, HDL vehicle, TRAF6i-HDL, mTORi-HDL and mTORi-HDL/TRAF6i-HDL combination therapy form FIG. 23. FIG. 49 shows in one aspect the therapeutic effects of combined mTORi-HDL and TRAF6i-HDL nanobiologics.

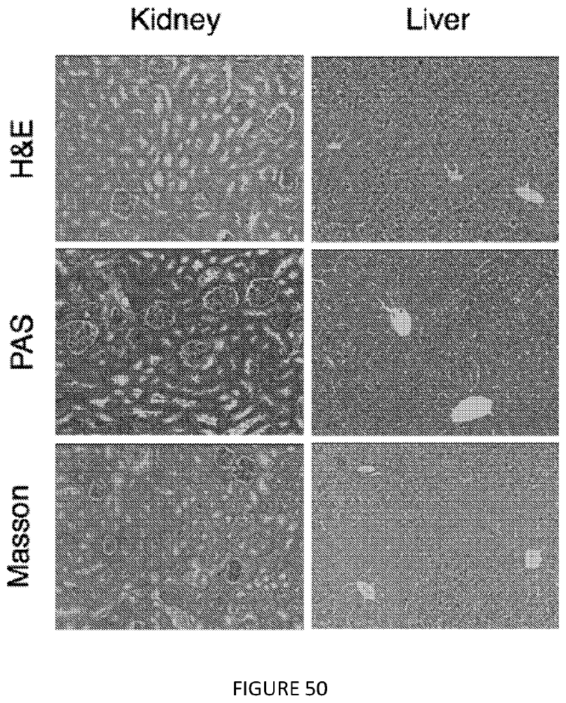

FIG. 50 is a six-panel illustration of representative kidney and liver immunohistochemical images for hematoxylin/eosin (H&E), Periodic Acid Schiff (PAS) and Masson Trichrome from mTORi/TRAF6i-HDL-treated transplant recipients collected at day 100 after transplantation. Kidney shows no significant changes in the three compartments of kidney parenchyma. Glomeruli appear normal, with no evidence of glomerulosclerosis. The tubules show no significant atrophy or any evidence of epithelial cell injury including vacuolization, loss of brush border or mitosis. Liver has normal acinar and lobular architecture. There is no evidence of inflammation or fibrosis in the portal tract and hepatic parenchyma. Hepatocytes are normal with no evidence of cholestasis, inclusions or apoptosis (n=4 mice; magnification ×200). FIG. 50 shows in one aspect the therapeutic effects of combined mTORi-HDL and TRAF6i-HDL nanobiologics.

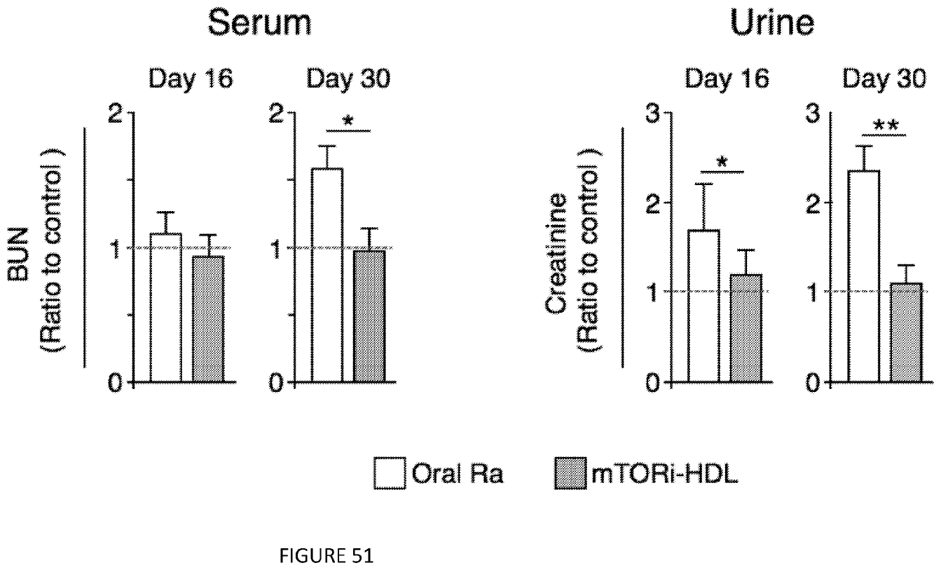

FIG. 51 is a pair of bar graph illustrations of toxicity associated with mTORi-HDL treatment. Recipient mice received either the mTORi-HDL treatment regimen (5 mg/kg on days 0 2, and 5 post-transplantation) or an oral rapamycin a treatment dose (5 mg/kg every day for 15 days) to achieve the same therapeutic outcome (100% allograft survival for 30 days). mTORi-HDL has no significant effects on blood urea nitrogen (BUN) or serum creatinine, but kidney toxicity parameters show statistical differences between oral rapamycin and mTORi-HDL. No differences between syngeneic and mTORi-HDL recipients were observed (n=4 mice/group, one-way ANOVA, *P<0.05; **P<0.01. Results from 3 experiments). FIG. 51 shows in one aspect the therapeutic effects of combined mTORi-HDL and TRAF6i-HDL nanobiologics.

### Atherosclerosis

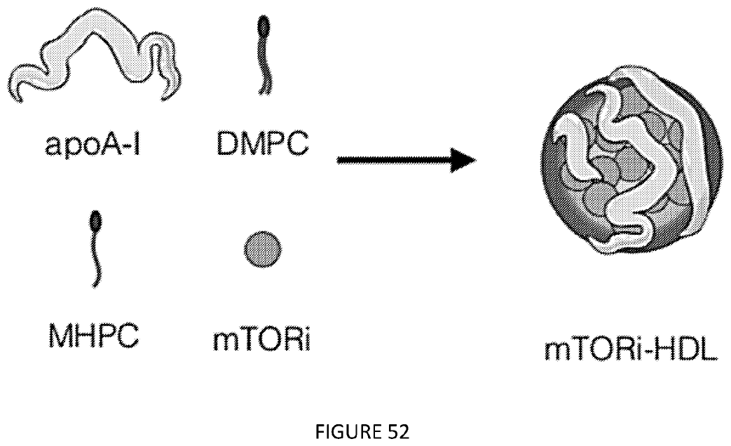

FIG. 52 is a schematic overview of the different components of mTORi-HDL, which was constructed by combining human apolipoprotein A-I (apoA-I), the phospholipids DMPC and MHPC, and the mTOR inhibitor rapamycin. FIG. 52 shows in one aspect that mTORi-HDL targets atherosclerotic plaques and accumulates in macrophages and inflammatory Ly6^{Chi} monocytes. Apoe−/− mice were on a high-cholesterol diet for 12 weeks to develop atherosclerotic plaques.

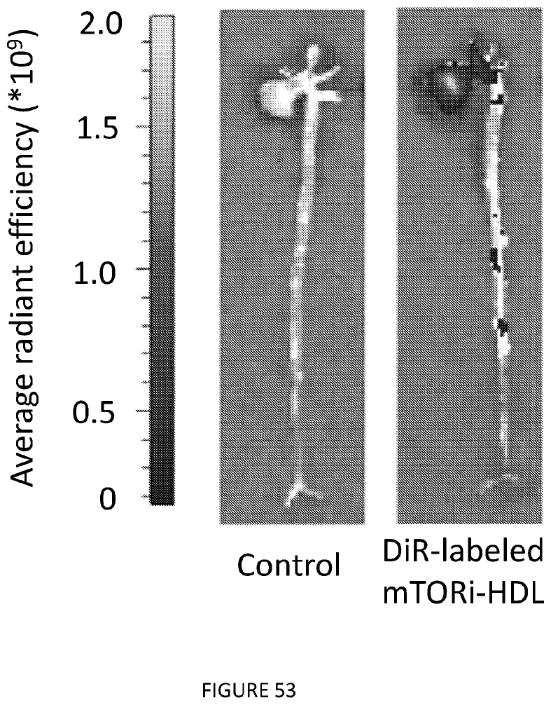

FIG. 53 is a graphic illustration in three-panels of IVIS imaging of whole aortas of Apoe−/− mice, injected with PBS (Control) or DiR-labeled mTORi-HDL. Aortas were harvested 24 hours after injection.

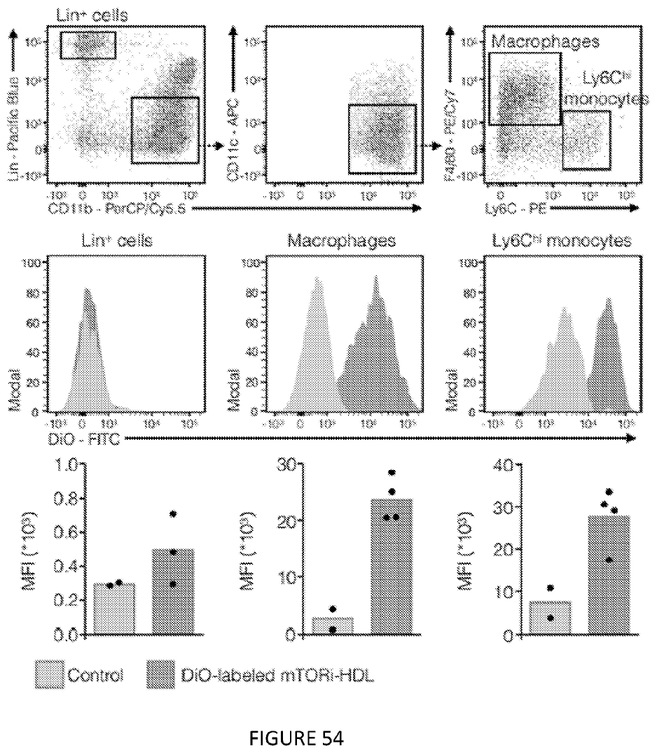

FIG. 54 is a graphic illustration in nine-panels of a flow cytometry gating strategy of CD45+ cells in the whole aorta. Identification of Lin+ cells, macrophages and Ly6Chi monocytes (top), representative histograms (middle) and quantification of DiO signal (bottom) in each cell type. Aortas were harvested 24 hours after injection of DiO-labeled mTORi-HDL. FIG. 54 shows in one aspect that mTORi-HDL targets atherosclerotic plaques and accumulates in macrophages and inflammatory Ly6Chi monocytes.

For all figures, data are presented as mean±SD. *p<0.05, **p<0.01, ***p<0.001. P values were calculated using Mann-Whitney U tests (two-sided).

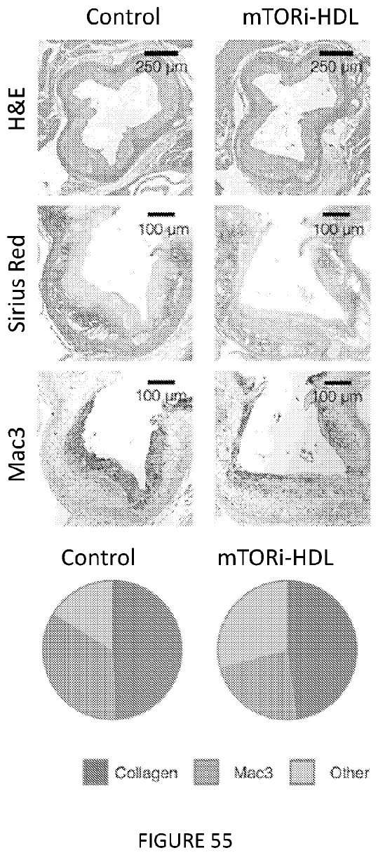

FIG. 55 is a graphical illustration of six-panels of histological images and two panels of pie charts comparing control group to mTORi-HDL.

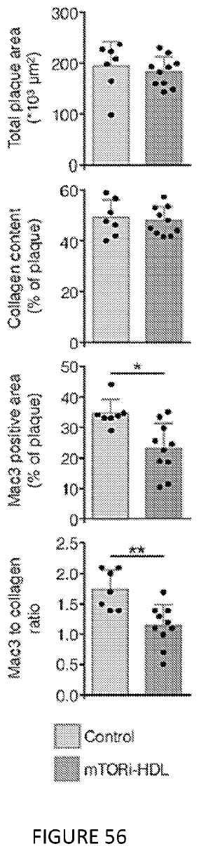

FIG. 56, right is a four-panel graphical illustration of plaque area, collagen content, Mac3 positive area, and Mac3 to collagen ratio, comparing Control to mTORi-HDL. FIG. 55-56 shows in one aspect that mTORi-HDL atherosclerotic plaque inflammation. Apoe−/− mice were on a high-cholesterol diet for 12 weeks, followed by 1 week of treatment, while kept on high-cholesterol diet.

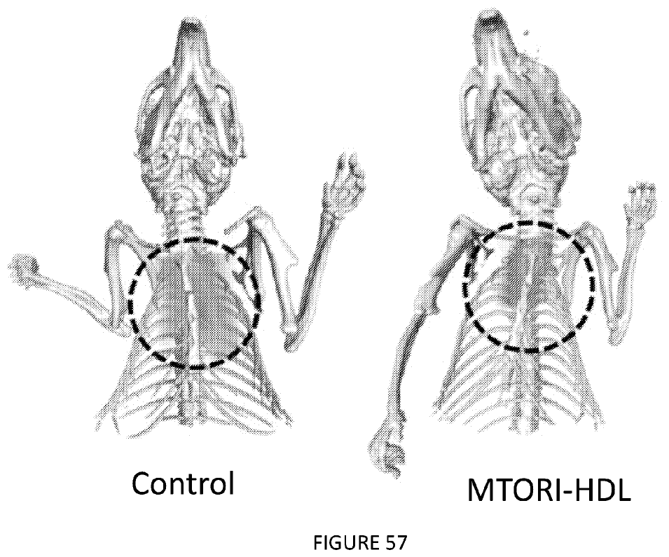

FIG. 57 is a pair of side-by-side fluorescence molecular tomography with X-ray computed tomography imaging showed decreased protease activity in the aortic root in mTORi-HDL treated mice vs control mice vs. mTORi-HDL mice showing significant reduction.

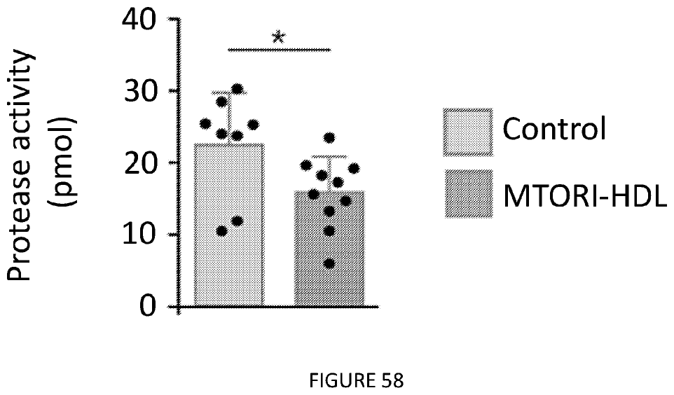

FIG. 58 is a graph of protease activity.

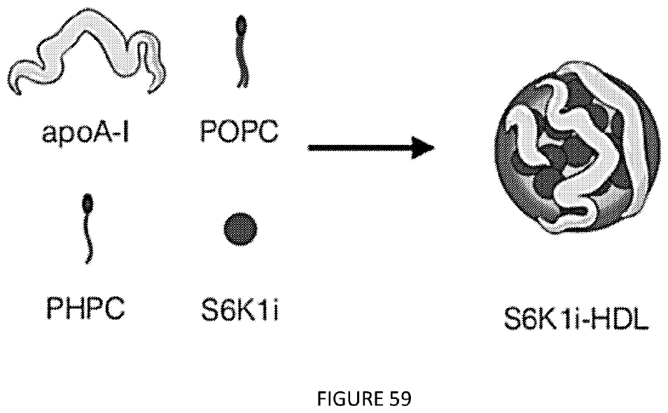

FIG. 59 is a schematic overview of the different components of the S6K1i-HDL nanobiologic, which was constructed by combining human apolipoprotein A-I (apoA-I), the phospholipidlipids POPC and PHPC, and the S6K1 inhibitor PF-4708671.

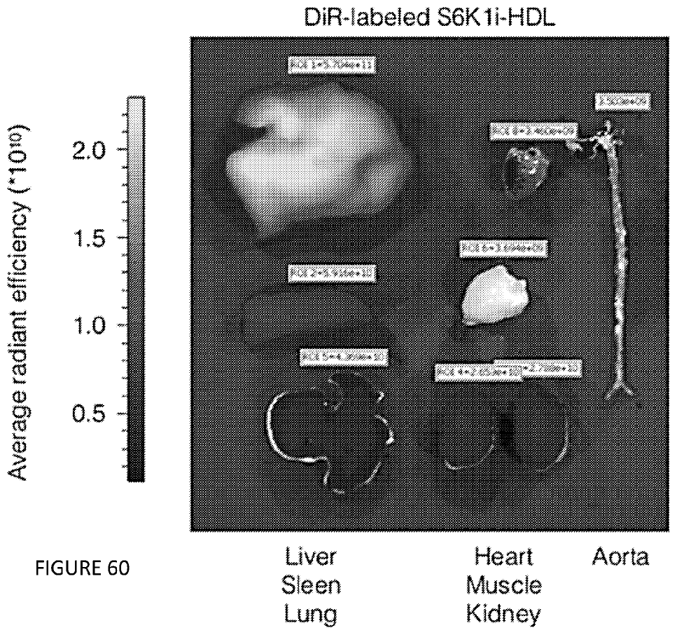

FIG. 60 is a graphical illustration of IVIS imaging of organs of Apoe−/− mice, injected with DiR-labeled S6K1i-HDL. Organs were harvested 24 hours after injection.

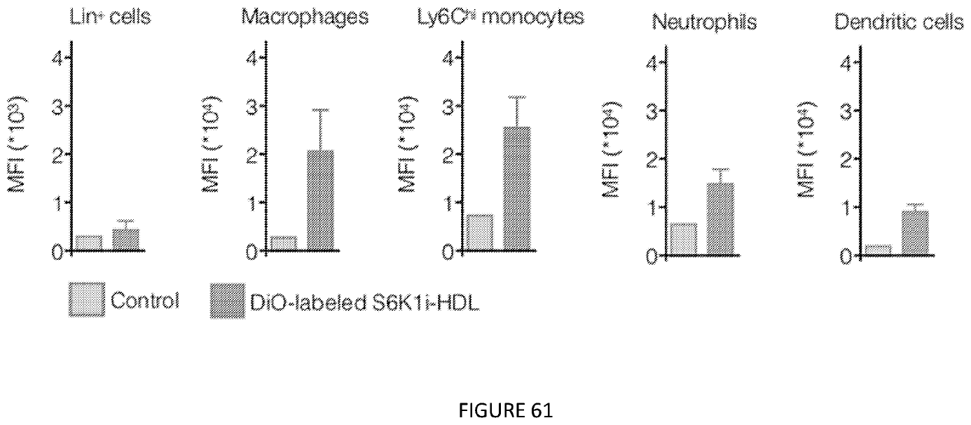

FIG. 61 is a five-panel graphical illustration of quantification of DiO signal of different leukocyte subsets in the aortic plaque after intravenous injection of DiO-labeled S6K1i-HDL (n=2-4 per group).

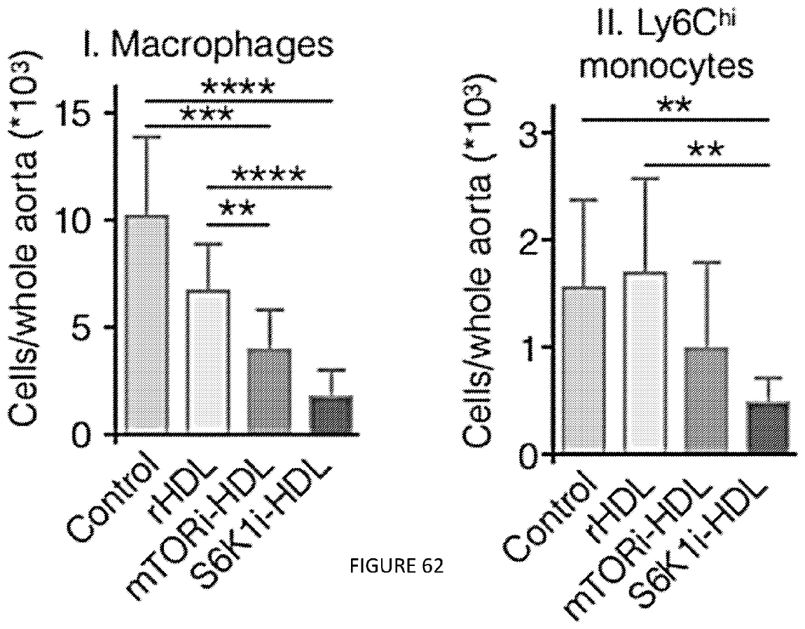

FIG. 62 is a pair of graphs of macrophage and Ly6C(hi) monocyte cell quantification in whole aorta and comparing control, rHDL only, mTORi-HDL, and S6K1i-HDL treatment. Apoe−/− mice were on a high-cholesterol diet for 12 weeks, followed by 1 week of treatment, while kept on high-cholesterol diet.

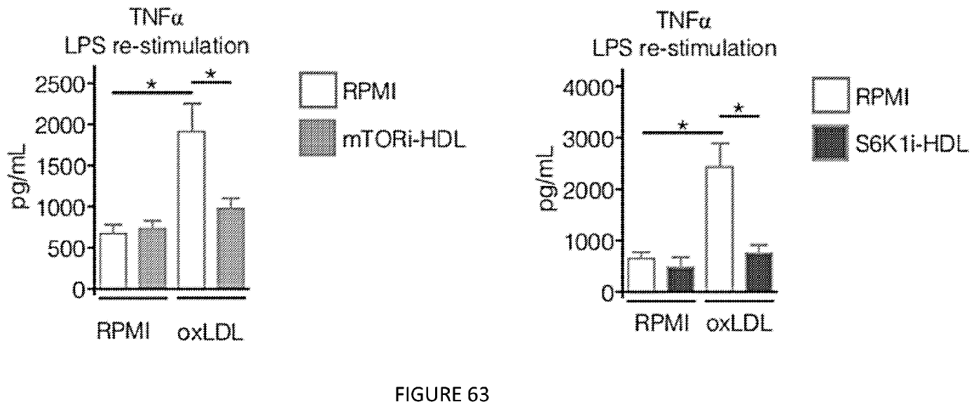

FIG. 63 shows in vitro analysis of human adherent monocytes in which trained immunity was induced by oxLDL, resulting in amplified TNFα cytokine production when cells are re-stimulated with LPS five days later. This response was mitigated by mTORi-HDL and S6K1i-HDL (n=6). FIG. 63 is a pair of graphs of TNFα levels in μg/mL for RPMI and oxLDL insult comparing RPMI alone vs. mTORi-HDL and RPMI alone vs. S6K1i-HDL.

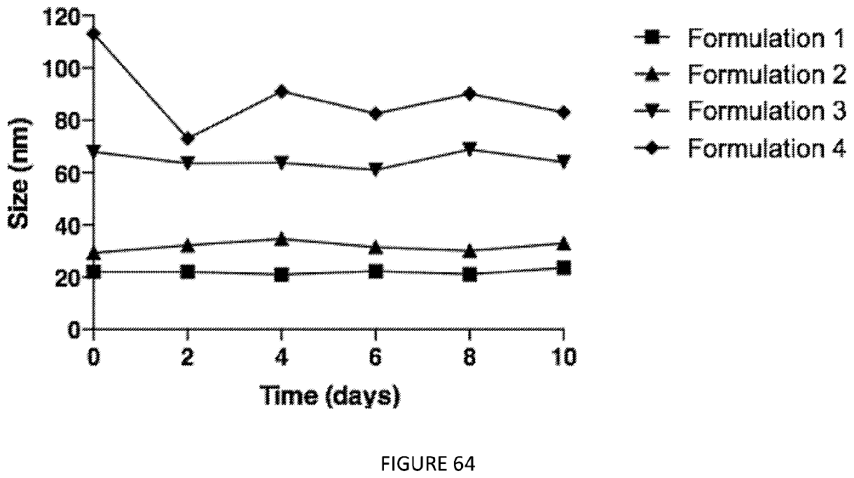

FIG. 64 is a graphical illustration of various formulations of prodrugs by size over time.

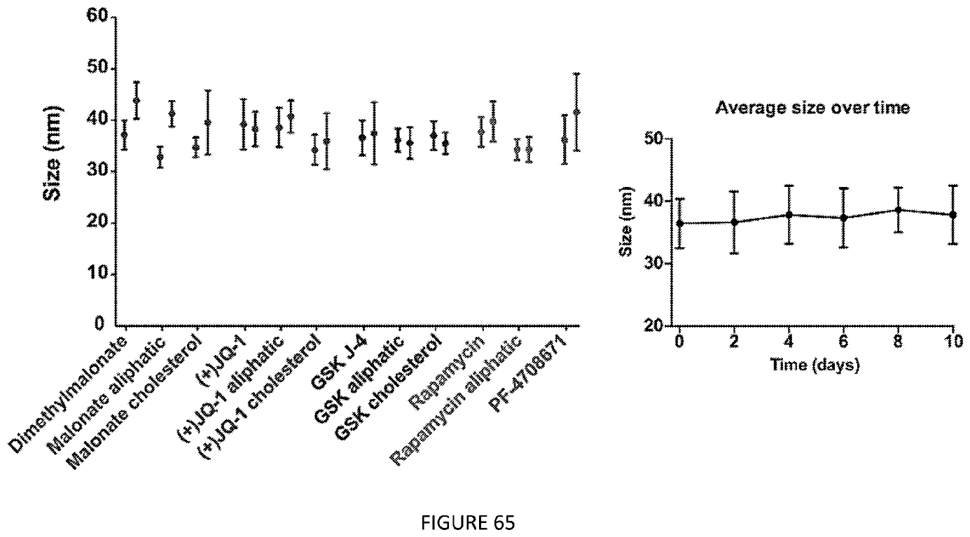

FIG. 65 is a graphical illustration of prodrug size over time.

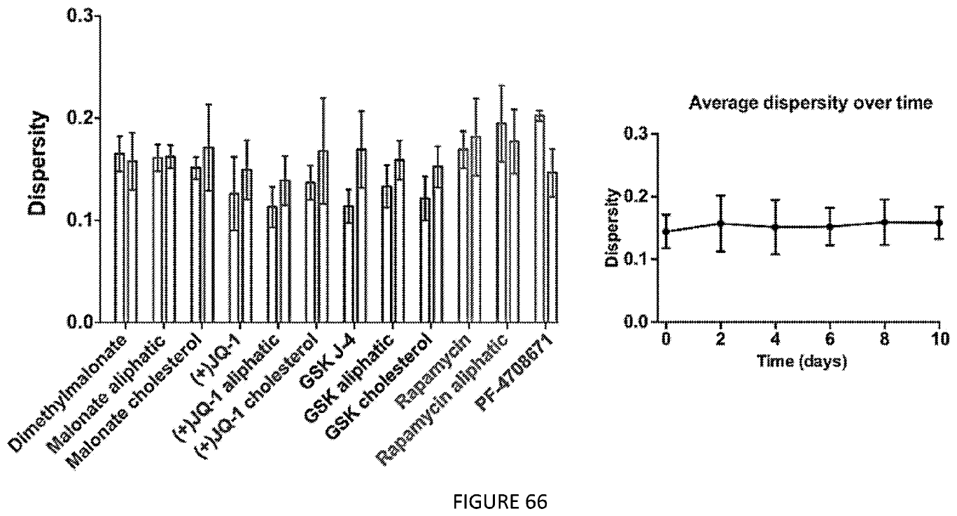

FIG. 66 is a graphical illustration of average dispersity of various prodrugs over time.

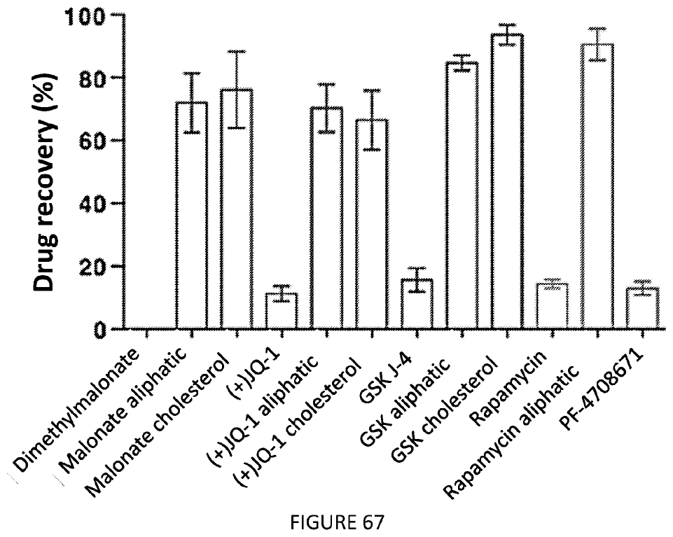

FIG. 67 is a graphical illustration of percent drug recovery of various prodrugs.

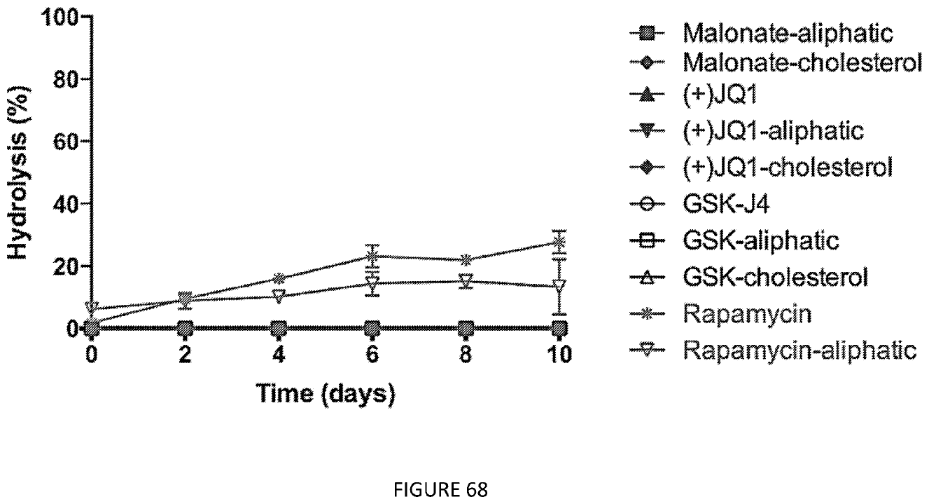

FIG. 68 is a graphical illustration of percent hydrolysis of various prodrugs.

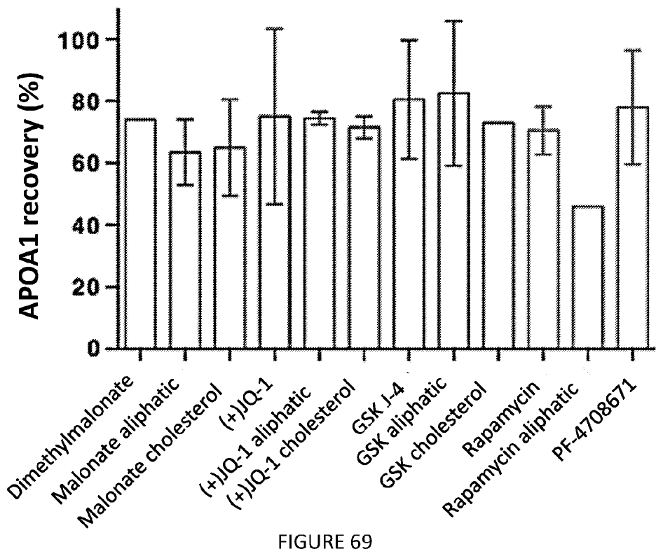

FIG. 69 is a graphical illustration of percent apoA-I recovery of various prodrugs.

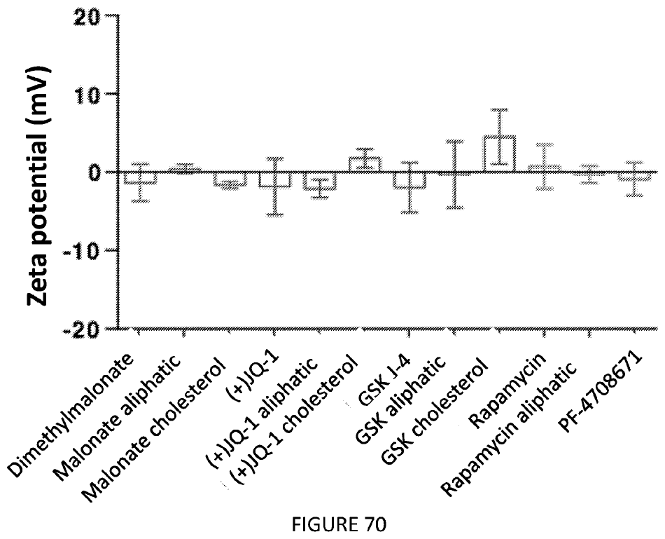

FIG. 70 is a graphical illustration of the Zeta potential of various prodrugs.

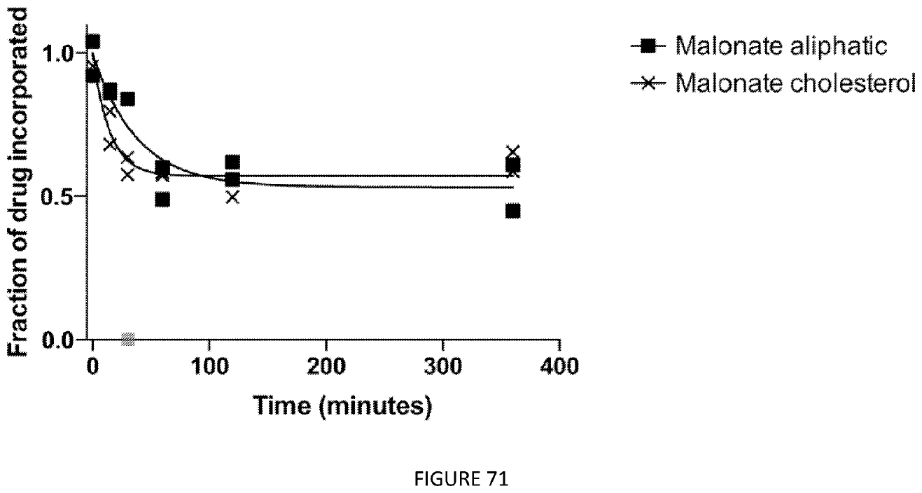

FIG. 71 is a graphical illustration of fraction of drug (Malonate) incorporated in aliphatic vs. cholesterol matrix.

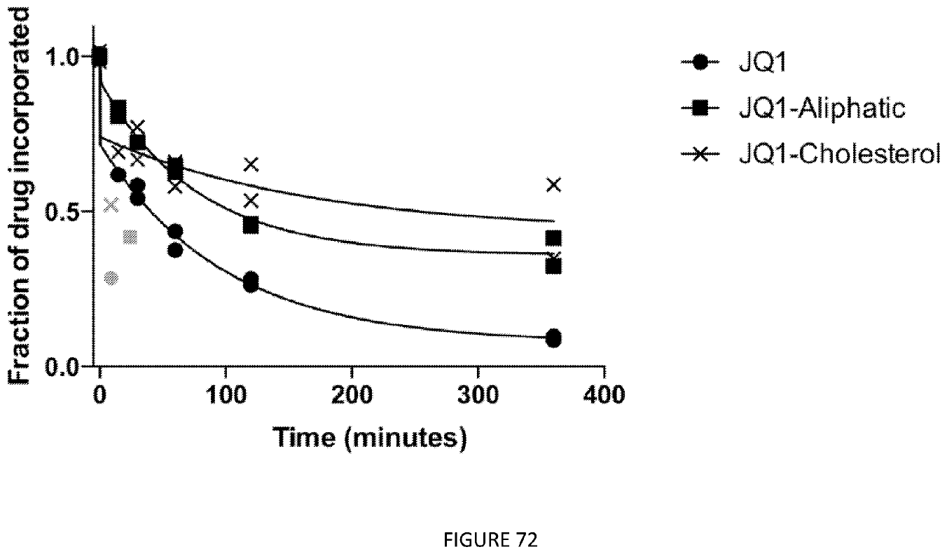

FIG. 72 is a graphical illustration of fraction of drug (JQ1) incorporated in aliphatic vs. cholesterol matrix.

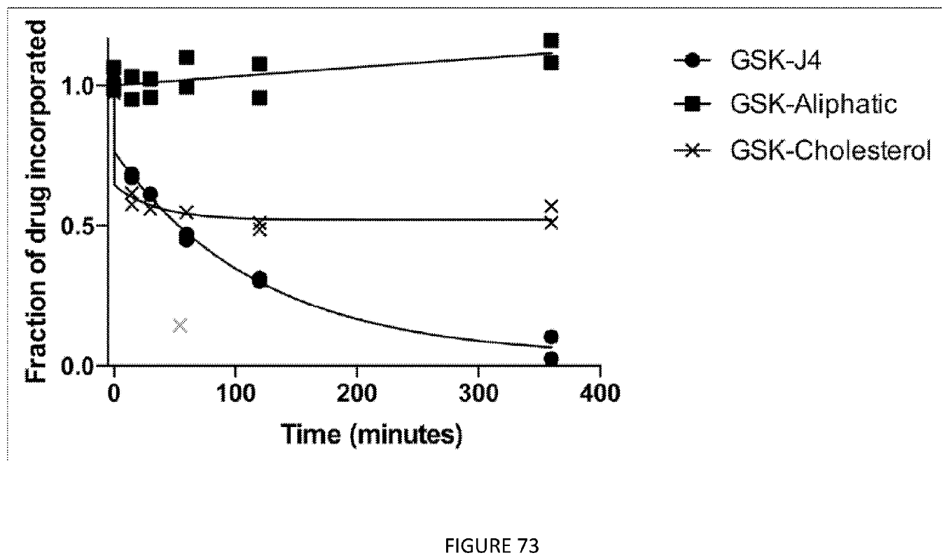

FIG. 73 is a graphical illustration of fraction of drug (GSK-J4) alone vs. incorporated in aliphatic vs. cholesterol matrix.

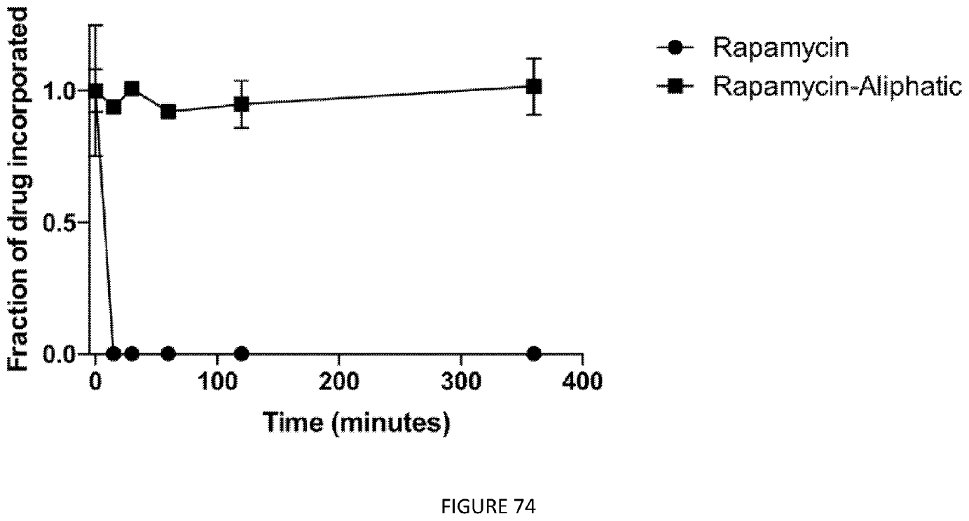

FIG. 74 is a graphical illustration of fraction of drug (Rapamycin) alone vs. incorporated in aliphatic.

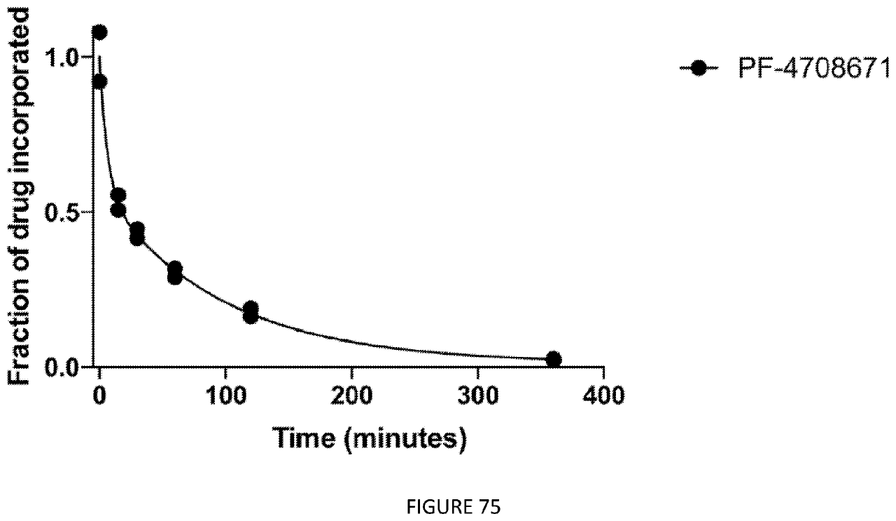

FIG. 75 is a graphical illustration of fraction of drug (PF-4708671 S6K1i) incorporated over time.

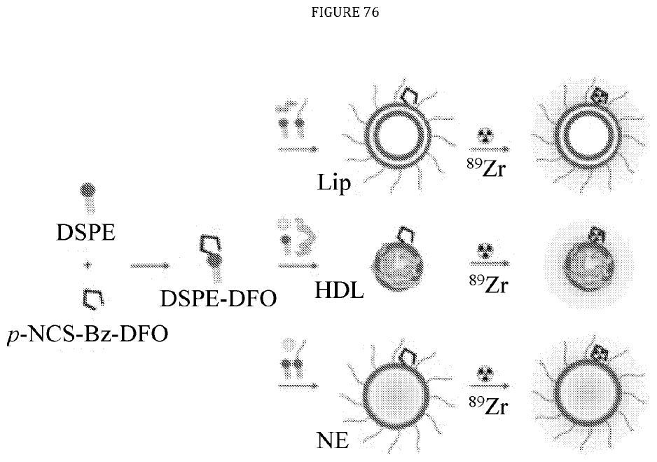

FIG. 76 is a graphic illustration of the radioisotope labeling process.

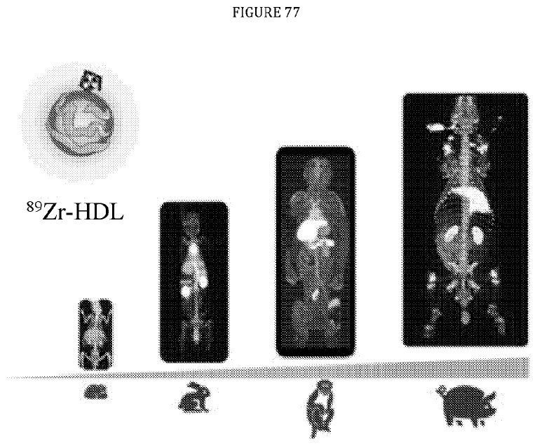

FIG. 77 is a graphic illustration of PET imaging using a radioisotope delivered by nanobiologic and shows accumulation of the nanobiologic in the bone marrow and spleen of a mouse, rabbit, monkey, and pig model.

### DETAILED DESCRIPTION OF THE INVENTION

The invention is directed to nanobiologic composition for inhibiting trained immunity, methods of making such nanobiologics, methods of incorporating drug into said nanobiologics, pro-drug formulations combining drug with functionalized linker moieties such as phospholipids, aliphatic chains, and sterols.

Inflammation is triggered by innate immune cells as a defense mechanism against tissue injury. An ancient mechanism of immunological memory, named trained immunity, also called innate immune memory, as defined by a long-term increased responsiveness (e.g. high cytokine production) after re-stimulation with a secondary stimulus of myeloid innate immune cells, being induced by a primary insult stimulating these cells or their progenitors and stem cells in the bone marrow, blood and/or spleen, and mediated by epigenetic, metabolic and transcriptional rewiring.

Trained Immunity is defined by a secondary long-term hyper-responsiveness, as manifested by increased cytokine excretion caused by the metabolic and epigenetic rewiring, to re-stimulation after a primary insult of the myeloid cells, the myeloid progenitors, and the hematopoietic stem cells in the bone marrow, blood, and/or spleen.

The invention is directed in one preferred embodiment to a myeloid cell-specific nanoimmunotherapy, based on delivering a nanobiologic carrying or having an incorporated mTOR inhibitor rapamycin (mTORi-HDL), which prevents epigenetic and metabolic modifications underlying trained immunity. The invention relates to therapeutic nanobiologic compositions and methods of treating patients who have had an organ transplant, or who suffer from atherosclerosis, arthritis, inflammatory bowel disease including Crohn's, autoimmune diseases including diabetes, and/or autoinflammatory conditions, or after a cardiovascular events, including stroke and myocardial infarction, by inhibiting trained immunity, which is the long-term increased responsiveness, the result of metabolic and epigenetic re-wiring of myeloid cells and their stem cells and progenitors in the bone marrow and spleen and blood induced by a primary insult, and characterized by increased cytokine excretion after re-stimulation with one or multiple secondary stimuli.

### Definitions

### Nanobiologic

The term “nanobiologic” refers to a composition for inhibiting trained immunity, comprising: a nanoscale assembly, and

(ii) an inhibitor drug incorporated in the nanoscale assembly,
wherein the nanoscale assembly is a multi-component carrier composition comprising: (a) a phospholipid or a mixture of phospholipids,
(b) apolipoprotein A-I (apoA-I) or a peptide mimetic of apoA-I,
and optionally including (c) a hydrophobic matrix composed of one or more triglycerides, fatty acid esters, hydrophobic polymers, and sterol esters, and
and optionally also including (d) cholesterol,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell.

For proof of concept, an inhibitor of mTOR incorporated into HDL (mTORi-HDL), or an inhibitor of S6K1 incorporated into HDL (S6K1i-HDL), functioned as a nanobiologic for generation of data herein.

### Nanoscale Assembly

The term “nanoscale assembly” (NA) refers to a multi-component carrier composition for carrying the active payload, e.g., drug.

In one preferred embodiment, the nanoscale assembly comprises a multi-component carrier composition for carrying the active payload having the subcomponents: (a) phospholipids, and (b) apolipoprotein A-I(apoA-I) or a peptide mimetic of apoA-I.

In another preferred embodiment, the “nanoscale assembly” (NA) refers to a multi-component carrier composition for carrying the trained immunity-inhibiting active payload, e.g. drug, having the subcomponents: (a) phospholipids, (b) apolipoprotein A-I (apoA-I) or a peptide mimetic of apoA-I, and (c) a hydrophobic matrix comprising one or more triglycerides, fatty acid esters, hydrophobic polymers, and sterol esters.

In another preferred embodiment, the “nanoscale assembly” (NA) refers to a multi-component carrier composition for carrying the trained immunity-inhibiting active payload, e.g. drug, having the subcomponents: (a) phospholipids, (b) apolipoprotein A-I (apoA-I) or a peptide mimetic of apoA-I, (c) a hydrophobic matrix comprising one or more triglycerides, fatty acid esters, hydrophobic polymers, and sterol esters, and (d) cholesterol.

### Phospholipids

The term “phospholipid” refers to an amphiphilic compound that consists of two hydrophobic fatty acid “tails” and a hydrophilic “head” consisting of a phosphate group.

The two components are joined together by a glycerol molecule. The phosphate groups can be modified with simple organic molecules such as choline, ethanolamine or serine. Choline refers to an essential, bioactive nutrient having the chemical formula R—(CH₂)₂—N—(CH₂)₄. When a phospho-moiety is R— it is called phosphocholine.

Examples of suitable phospholipids include, without limitation, phosphatidylcholines, phosphatidylethanolamines, phosphatidylinositol, phosphatidylserines, sphingomyelin or other ceramides, as well as phospholipid-containing oils such as lecithin oils. Combinations of phospholipids, or mixtures of a phospholipid(s) and other substance(s), may be used.

Non-limiting examples of the phospholipids that may be used in the present composition include phosphatidylcholines (PC), phosphatidylglycerols (PG), phosphatidylserines (PS), phosphatidylethanolamines (PE), and phosphatidic acid/esters (PA), and lysophosphatidylcholines.

Specific examples include: DDPC CAS-3436-44-0 1,2-Didecanoyl-sn-glycero-3-phosphocholine, DEPA-NA CAS-80724-31-8 1,2-Dierucoyl-sn-glycero-3-phosphate (Sodium Salt), DEPC CAS-56649-39-9 1,2-Dierucoyl-sn-glycero-3-phosphocholine, DEPE CAS-988-07-2 1,2-Dierucoyl-sn-glycero-3-phosphoethanolamine, DEPG-NA 1,2-Dierucoyl-sn-glycero-3[Phospho-rac-(1-glycerol . . . ) (Sodium Salt), DLOPC CAS-998-06-1 1,2-Dilinoleoyl-sn-glycero-3-phosphocholine, DLPA-NA 1,2-Dilauroyl-sn-glycero-3-phosphate (Sodium Salt), DLPC CAS-18194-25-7 1,2-Dilauroyl-sn-glycero-3-phosphocholine, DLPE 1,2-Dilauroyl-sn-glycero-3-phosphoethanolamine, DLPG-NA 1,2-Dilauroyl-sn-glycero-3[Phospho-rac-(1-glycerol . . . ) (Sodium Salt), DLPG-NH4 1,2-Dilauroyl-sn-glycero-3[Phospho-rac-(1-glycerol . . . ) (Ammonium Salt), DLPS-NA 1,2-Dilauroyl-sn-glycero-3-phosphoserine (Sodium Salt), DMPA-NA CAS-80724-3 1,2-Dimyristoyl-sn-glycero-3-phosphate (Sodium Salt), DMPC CAS-18194-24-6 1,2-Dimyristoyl-sn-glycero-3-phosphocholine, DMPE CAS-988-07-2 1,2-Dimyristoyl-sn-glycero-3-phosphoethanolamine, DMPG-NA CAS-67232-80-8 1,2-Dimyristoyl-sn-glycero-3[Phospho-rac-(1-glycerol . . . ) (Sodium Salt), DMPG-NH4 1,2-Dimyristoyl-sn-glycero-3[Phospho-rac-(1-glycerol . . . ) (Ammonium Salt), DMPG-NH4/NA 1,2-Dimyristoyl-sn-glycero-3[Phospho-rac-(1-glycerol . . . ) (Sodium/Ammonium Salt), DMPS-NA 1,2-Dimyristoyl-sn-glycero-3-phosphoserine (Sodium Salt), DOPA-NA 1,2-Dioleoyl-sn-glycero-3-phosphate (Sodium Salt), DOPC CAS-4235-95-4 1,2-Dioleoyl-sn-glycero-3-phosphocholine, DOPE CAS-4004-5-1 1,2-Dioleoyl-sn-glycero-3-phosphoethanolamine, DOPG-NA CAS-62700-69-0 1,2-Dioleoyl-sn-glycero-3[Phospho-rac-(1-glycerol . . . )(Sodium Salt), DOPS-NA CAS-70614-14-1 1,2-Dioleoyl-sn-glycero-3-phosphoserine (Sodium Salt), DPPA-NA CAS-71065-87-7 1,2-Dipalmitoyl-sn-glycero-3-phosphate (Sodium Salt), DPPC CAS-63-89-8 1,2-Dipalmitoyl-sn-glycero-3-phosphocholine, DPPE CAS-923-61-5 1,2-Dipalmitoyl-sn-glycero-3-phosphoethanolamine, DPPG-NA CAS-67232-81-9 1,2-Dipalmitoyl-sn-glycero-3[Phospho-rac-(1-glycerol . . . ) (Sodium Salt), DPPG-NH4 CAS-73548-70-6 1,2-Dipalmitoyl-sn-glycero-3[Phospho-rac-(1-glycerol . . . ) (Ammonium Salt), DPPS-NA 1,2-Dipalmitoyl-sn-glycero-3-phosphoserine (Sodium Salt), DSPA-NA CAS-108321-18-2 1,2-Distearoyl-sn-glycero-3-phosphate (Sodium Salt), DSPC CAS-816-94-4 1,2-Distearoyl-sn-glycero-3-phosphocholine, DSPE CAS-1069-79-0 1,2-Distearoyl-sn-glycero-3-phosphoethanolamine, DSPG-NA CAS-67232-82-0 1,2-Distearoyl-sn-glycero-3[Phospho-rac-(1-glycerol . . . ) (Sodium Salt), DSPG-NH4 CAS-108347-80-4 1,2-Distearoyl-sn-glycero-3[Phospho-rac-(1-glycerol . . . ) (Ammonium Salt), DSPS-NA 1,2-Distearoyl-sn-glycero-3-phosphoserine (Sodium Salt), EPC Egg-PC, HEPC Hydrogenated Egg PC, HSPC Hydrogenated Soy PC, LYSOPC MYRISTIC CAS-18194-24-6 1-Myristoyl-sn-glycero-3-phosphocholine, LYSOPC PALMITIC CAS-17364-16-8 1-Palmitoyl-sn-glycero-3-phosphocholine, LYSOPC STEARIC CAS-19420-57-6 1-Stearoyl-sn-glycero-3-phosphocholine, Milk Sphingomyelin, MPPC 1-Myristoyl-2-palmitoyl-sn-glycero 3-phosphocholine, MSPC 1-Myristoyl-2-stearoyl-sn-glycero-3-phosphocholine, PMPC 1-Palmitoyl-2-myristoyl-sn-glycero-3-phosphocholine, POPC CAS-26853-31-6 1-Palmitoyl-2-oleoyl-sn-glycero-3-phosphocholine, POPE 1-Palmitoyl-2-oleoyl-sn-glycero-3-phosphoethanolamine, POPG-NA CAS-81490-05-3 1-Palmitoyl-2-oleoyl-sn-glycero-3[Phospho-rac-(1-glycerol) . . . ] (Sodium Salt), PSPC 1-Palmitoyl-2-stearoyl-sn-glycero-3-phosphocholine, SMPC 1-Stearoyl-2-myristoyl-sn-glycero-3-phosphocholine, SOPC 1-Stearoyl-2-oleoyl-sn-glycero-3-phosphocholine, SPPC 1-Stearoyl-2-palmitoyl-sn-glycero-3-phosphocholine In some preferred embodiments, specific non-limiting examples of phospholipids include: dimyristoylphosphatidylcholine (DMPC), soy lecithin, dipalmitoylphosphatidylcholine (DPPC), distearoylphosphatidylcholine (DSPC), diaurylolyphosphatidylcholine (DLPC), dioleoylphosphatidylcholine (DOPC), dilaurylolylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dimyristoyl phosphatidic acid (DMPA), dimyristoyl phosphatidic acid (DMPA), dipalmitoyl phosphatidic acid (DPPA), dipalmitoyl phosphatidic acid (DPPA), dimyristoyl phosphatidylethanolamine (DMPE), dipalmitoyl phosphatidylethanolamine (DPPE), dimyristoyl phosphatidylserine (DMPS), dipalmitoyl phosphatidylserine (DPPS), dipalmitoyl sphingomyelin (DPSP), distearoyl sphingomyelin (DSSP), and mixtures thereof.

In certain embodiments, when the present composition comprises (consists essentially of, or consists of) two or more types of phospholipids, the weight ratio of two types of phospholipids may range from about 1:10 to about 10:1, from about 2:1 to about 4:1, from about 1:1 to about 5:1, from about 2:1 to about 5:1, from about 6:1 to about 10:1, from about 7:1 to about 10:1, from about 8:1 to about 10:1, from about 7:1 to about 9:1, or from about 8:1 to about 9:1. For example, the weight ratio of two types of phospholipids may be about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1.

In one embodiment, the (a) phospholipids of the present nanoscale assembly comprise (consists essentially of, or consists of) a mixture of a two-chain diacyl-phospholipid and a single chain acyl-phospholipid/lysolipid.

In one embodiment, the (a) phospholipids is a mixture of phospholipid and lysolipid is (DMPC), and (MHPC).

The weight ratio of DMPC to MHPC may range from about 1:10 to about 10:1, from about 2:1 to about 4:1, from about 1:1 to about 5:1, from about 2:1 to about 5:1, from about 6:1 to about 10:1, from about 7:1 to about 10:1, from about 8:1 to about 10:1, from about 7:1 to about 9:1, or from about 8:1 to about 9:1. The weight ratio of DMPC to MHPC may be about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1.

In one embodiment, the (a) phospholipids is a mixture of phospholipid and lysolipid is (POPC) and (PHPC).

The weight ratio of POPC to PHPC may range from about 1:10 to about 10:1, from about 2:1 to about 4:1, from about 1:1 to about 5:1, from about 2:1 to about 5:1, from about 6:1 to about 10:1, from about 7:1 to about 10:1, from about 8:1 to about 10:1, from about 7:1 to about 9:1, or from about 8:1 to about 9:1. The weight ratio of DMPC to MHPC may be about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1.

It is noted that all phospholipids ranging in chain length from C4 to C30, saturated or unsaturated, cis or trans, unsubstituted or substituted with 1-6 side chains, and with or without the addition of lysolipids are contemplated for use in the nanoscale assembly or nanoparticles/nanobiologics described herein.

Additionally, other synthetic variants and variants with other phospholipid headgroups are also contemplated.

### Lysolipids

The term “lysolipids” as used herein, include (acyl-, single chain) such as in non-limiting embodiments 1-myristoyl-2-hydroxy-sn-glycero-3-phosphocholine (MHPC), 1-Palmitoyl-2-hexadecyl-sn-glycero-3-phosphocholine (PHPC) and 1-stearoyl-2-hydroxy-sn-glycero-3-phosphocholine (SHPC).

### Apolipoprotein A-I(Apoa-I) (ApoA1)

The term “apolipoprotein A-I” or “apoA-I”, and also “apoliprotein A1” or “apoA1”, refers to a protein that is encoded by the APOA1 gene in humans, and as used herein also includes peptide mimetics of apoA-I. Apolipoprotein A1 (apoA-I) is subcomponent (b) in the nanoscale assembly.

### Hydrophobic Matrix

The term“hydrophobic matrix” refers to a core or filler or structural modifier of the nanobiologic. Structural modifications include (1) using the hydrophobic matrix to increase or design the particle size of a nanoscale assembly made from only (a) phospholipids and (b) apoA-I, (2) increasing or decreasing (designing) the size and/or shape of the nanoscale assembly particles, (3) increasing or decreasing (designing) the hydrophobic core of nanoscale assembly particles, (4) increasing or decreasing (designing) the nanobiologic's capacity to incorporate hydrophobic drugs, and/or miscibility, and (5) increasing or decreasing the biodistribution characteristics of the nanoscale assembly particles. Nanoscale assembly particle size, rigidity, viscosity, and/or biodistribution, can be moderated by the quantity and type of hydrophobic molecule added. In a non-limiting example, a nanoscale assembly made from only (a) phospholipids and (b) apoA-I may have a diameter of 10 nm-50 nm. Adding (c) a hydrophobic matrix molecule such as triglycerides, swells the nanoscale assembly from a minimum of 10 nm to at least 30 nm. Adding more triglycerides can increase the diameter of the nanoscale assembly to at least 50 nm, at least 75 nm, at least 100 nm, at least 150 nm, at least 200 nm, at least 300 nm, and up to 400 nm within the scope of the invention.

Production methods can prepare uniform size nanoscale assembly particles, or a non-uniform sized mixture of nanoscale assembly particles, either by not filtering, or by preparing a range of different sized nanoscale assembly particles and re-combining them in a post-production step. The larger the size of the nanoscale assembly particles, the more drug can be incorporated. However, larger sizes e.g. >120 nm, can limit, prevent or slow diffusion of the nanoscale assembly particles into the tissues of the patient being treated. Smaller nanoscale assembly particles do not hold as much drug per particle, but are able to access the bone marrow, blood, or spleen, or other localized tissue affected by trained immunity, e.g. transplant and surrounding tissues, atherosclerotic plaque, and so forth (biodistribution). Using a non-uniform mixture of nanoparticles sizes in a single administration or regimen can produce an immediate reduction in innate immune hyper-responsiveness, and simultaneously produce a durable, long-term reduction in innate immune hyper-responsiveness that can last days, weeks, months, and years, wherein the nanobiologic has reversed, modified, or re-regulated the metabolic, epigenetic, and inflammasome pathways of the hematopoietic stem cells (HSC), the common myeloid progenitors (CMP), and the myeloid cells such as monocytes, macrophages and other short-lived circulating cells.

Adding other (c) hydrophobic matrix molecules, such as cholesterol, fatty acid esters, hydrophobic polymers, sterol esters, and different types of triglycerides, or specific mixtures thereof, can further design the nanoscale assembly particles to emphasize specific desired characteristics for specific purposes. Size, rigidity, and viscosity can affect loading and biodistribution.

By way of non-limiting example, maximum loading capacity can be determined dividing the volume of the interior of the nanoscale assembly particle by the volume of a drug-load spheroid.

Particle: assume a 100 nm spherical particle having 2.2 nm-3.0 nm phospholipid wall, yielding a 94 nm diameter interior with Volume (L) @ 4/3π(r)3.

Drug: assume sirolimus (Rapamycin) at 12×12×35 Angstrom or as a cylinder 1.2×1.2×3.5 nm, where multiple drug molecule cylinders, e.g. seven or nine, etc., or multiple drug+hydrophobic matrix carrier such as a triglyeride, could assume a 3.5 nm diameter spheroid having a radius of 1.75 nm Vol(small) @ 4/3π(r)3.

Maximum Loading Capacity (calc): ˜19,372 3.5 nm spheroids within a 100 nm particle.

Biologically relevant lipids include fatty acyls, glycerolipids, glycerophospholipids, sphingolipids, sterol lipids, prenol lipids, saccharolipids, and polyketides. A complete list of over 42,000 lipids can be obtained at https://www.lipidmaps.org.

### Triglyceride

“Triglyceride” and like terms mean an ester derived from glycerol and three fatty acids. The notation used in this specification to describe a triglyceride is the same as that used below to describe a fatty acid. The triglyceride can comprise glycerol with any combination of the following fatty acids: C18:1, C14:1, C16:1, polyunsaturated, and saturated. Fatty acids can attach to the glycerol molecule in any order, e.g., any fatty acid can react with any of the hydroxyl groups of the glycerol molecule for forming an ester linkage. Triglyceride of C18:1 fatty acid simply means that the fatty acid components of the triglyceride are derived from or based upon a C18:1 fatty acid. That is, a C18:1 triglyceride is an ester of glycerol and three fatty acids of 18 carbon atoms each with each fatty acid having one double bond. Similarly, a C14:1 triglyceride is an ester of glycerol and three fatty acids of 14 carbon atoms each with each fatty acid having one double bond. Likewise, a C16:1 triglyceride is an ester of glycerol and three fatty acids of 16 carbon atoms each with each fatty acid having one double bond. Triglycerides of C18:1 fatty acids in combination with C14:1 and/or C16:1 fatty acids means that: (a) a C18:1 triglyceride is mixed with a C14:1 triglyceride or a C16:1 triglyceride or both; or (b) at least one of the fatty acid components of the triglyceride is derived from or based upon a C18:1 fatty acid, while the other two are derived from or based upon C14:1 fatty acid and/or C16:1 fatty acid.

### Fatty Acid

“Fatty acid” and like terms mean a carboxylic acid with a long aliphatic tail that is either saturated or unsaturated. Fatty acids may be esterified to phospholipids and triglycerides. As used herein, the fatty acid chain length includes from C4 to C30, saturated or unsaturated, cis or trans, unsubstituted or substituted with 1-6 side chains. Unsaturated fatty acids have one or more double bonds between carbon atoms. Saturated fatty acids do not contain any double bonds. The notation used in this specification for describing a fatty acid includes the capital letter “C” for carbon atom, followed by a number describing the number of carbon atoms in the fatty acid, followed by a colon and another number for the number of double bonds in the fatty acid. For example, C16:1 denotes a fatty acid of 16 carbon atoms with one double bond, e.g., palmitoleic acid. The number after the colon in this notation neither designates the placement of the double bond(s) in the fatty acid nor whether the hydrogen atoms bonded to the carbon atoms of the double bond are cis to one another. Other examples of this notation include C18:0 (stearic acid), C18:1 (oleic acid), C18:2 (linoleic acid), C18:3 (a-linolenic acid) and C20:4 (arachidonic acid).

### Sterols and Sterolesters

The term “Sterols” such as, but not limited to cholesterol, can also be utilized in the methods and compounds described herein. Sterols are animal or vegetable steroids which only contain a hydroxyl group but no other functional groups at C-3. In general, sterols contain 27 to 30 carbon atoms and one double bond in the 5/6 position and occasionally in the 7/8, 8/9 or other positions. Besides these unsaturated species, other sterols are the saturated compounds obtainable by hydrogenation. One example of a suitable animal sterol is cholesterol. Typical examples of suitable phytosterols, which are preferred from the applicational point of view, are ergosterols, campesterols, stigmasterols, brassicasterols and, preferably, sitosterols or sitostanols and, more particularly, β-sitosterols or β-sitostanols. Besides the phytosterols mentioned, their esters are preferably used. The acid component of the ester may go back to carboxylic acids corresponding to formula (I):

RiCO—OH (I)

in which RICO is an aliphatic, linear or branched acyl group containing 2 to 30 carbon atoms and 0 and/or 1, 2 or 3 double bonds. Typical examples are acetic acid, propionic acid, butyric acid, valeric acid, caproic acid, caprylic acid, 2-ethyl hexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, conjugated linoleic acid (CLA), linolenic acid, elaeosteric add, arachic acid, gadoleic acid, behenic acid and erucic acid.

### Hydrophobic Polymers

The hydrophobic polymer or polymers used to make up the matrix may be selected from the group of polymers approved for human use (i.e. biocompatible and FDA-approved). Such polymers comprise, for example, but are not limited to the following polymers, derivatives of such polymers, co-polymers, block co-polymers, branched polymers, and polymer blends: polyalkenedicarboxlates, polyanhydrides, poly(aspartic acid), polyamides, polybutylenesuccinates (PBS), polybutylenesuccinates-co-adipate (PBSA), poly(ε-caprolactone) (PCL), polycarbonates including poly-alkylene carbonates (PC), polyesters including aliphatic polyesters and polyester-amides, polyethylenesuccinates (PES), polyglycolides (PGA), polyimines and polyalkyleneimines (PI, PAI), polylactides (PLA, PLLA, PDLLA), polylactic-co-glycolic acid (PLGA), poly(l-lysine), polymethacrylates, polypeptides, polyorthoesters, poly-p-dioxanones (PPDO), (hydrophobic) modified-polysaccharides, polysiloxanes and poly-alkyl-siloxanes, polyureas, polyurethanes, and polyvinyl alcohols.

### Biohydrolyzable

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

### Method of Producing the Nanoscale Assembly

Methods are described below, and there are variations relating to these methods.

### Method 1—Film

The phospholipids, (pro-)drug and optional triglycerides or polymer are dissolved (typically in chloroform, ethanol or acetonitrile). This solution is then evaporated under vacuum to form a film of the components. Subsequently, a buffer solution is added to hydrate the film and generate a vesicle suspension.

The phospholipids, (pro-)drug and optional triglycerides or polymer are dissolved (typically in chloroform, ethanol or acetonitrile). This solution is infused—or added drop-wise—to a mildly heated buffer solution under stirring, until complete evaporation of the organic solvents, generating a vesicle suspension.

To the vesicle suspension, generated using A or B, apolipoprotein A-I(apoA-I) (note that apoA-I can also already be in B)—use dropwise to avoid denature, is added and the resulting mixture is sonicated for 30 minutes using a tip sonicator while being thoroughly cooled using an external ice-water bath. The obtained solution containing the nanobiologics and other by products is transferred to a Sartorius Vivaspin tube with a molecular weight cut-off depending on the estimated size of the nanobiologics (typically Vivaspin tubes with cut-offs of 10.000-100.000 kDa are used). The tubes are centrifuged until ˜90% of the solvent volume has passed through the filter. Subsequently, a volume of buffer, roughly equal to the volume of the remaining solution, is added and the tubes are spun again until roughly half the volume has passed through the filter. This is repeated twice after which the remaining solution is passed through a polyethersulfone 0.22 m syringe filter, resulting in the final nanobiologic solution.

### Method 2—Microfluidics

In an alternative approach, the phospholipids, (pro-)drug and optional triglycerides, cholesterol, steryl esters, or polymer are dissolved (typically in ethanol or acetonitrile) and loaded into a syringe. Additionally, a solution of apolipoprotein A-I (apoA-I) in phosphate buffered saline is loaded into a second syringe. Using microfluidies pumps, the content of both syringes is mixed using a microvortex platform. The obtained solution containing the nanobiologics and other by products is transferred to a Sartorius Vivaspin tube with a molecular weight cut-off depending on the estimate size of the particles (typically Vivaspin tubes with cut-offs of 10.000-100.000 kDa are used). The tubes are centrifuged until ˜90% of the solvent volume has passed through the filter. Subsequently, a volume of phosphate buffered saline roughly equal to the volume of the remaining solution is added and the tubes are spun again until roughly half the volume has passed through the filter. This is repeated twice after which the remaining solution is passed through a polyethersulfone 0.22 m syringe filter, resulting in the final nanobiologic solution.

### Method 3—Microfluidizer

In another preferred method according to the invention, microfluidizer technology is used to prepare the nanoscale assembly and the final nanobiologic composition.

Microfluidizers are devices for preparing small particle size materials operating on the submerged jet principle. In operating a microfluidizer to obtain nanoparticulates, a premix flow is forced by a high pressure pump through a so-called interaction chamber consisting of a system of channels in a ceramic block which split the premix into two streams. Precisely controlled shear, turbulent and cavitational forces are generated within the interaction chamber during microfluidization. The two streams are recombined at high velocity to produce shear. The so-obtained product can be recycled into the microfluidizer to obtain smaller and smaller particles.

Advantages of microfluidization over conventional milling processes include substantial reduction of contamination of the final product, and the ease of production scaleup.

### Microfluidizer Example 1-1L

Formation of Nanoscale Assembly and Rapamycin Nanobiologic

This example demonstrates the preparation of a pharmaceutical composition comprising rapamycin and the nanoscale assembly in which the rapamycin concentration is 4-8 mg/mL in the nanoscale assembly/emulsion and the formulation is made on a 1 L scale.

Rapamycin (7200 mg) is dissolved in 36 mL of chloroform/t-butanol. The solution is then added into 900 mL of a nanoscale assembly solution (3% w/v) including a mixture of POPC/PHPC phospholipids, apoA-I, tricaprylin, and cholesterol. The mixture is homogenized for 5 minutes at 10,000-15,000 rpm (Vitris homogenizer model Tempest I.Q.) in order to form a crude emulsion, and then transferred into a high pressure homogenizer. The emulsification is performed at 20,000 psi while recycling the emulsion. The resulting system is transferred into a Rotavap, and the solvent is rapidly removed at 40° C. at reduced pressure (25 mm of Hg). The resulting dispersion is translucent. The dispersion is serially filtered through multiple filters. The size of the filtered formulation is 8-400 nm.

### Microfluidizer Example 2-5L

Formation of Nanoscale Assembly and Rapamycin Nanobiologic

This example demonstrates the preparation of a pharmaceutical composition comprising rapamycin and the nanoscale assembly and the formulation is made on a 5 L scale. Rapamycin is dissolved in chloroform/t-butanol. The solution is then added into a nanoscale assembly solution (1-5% w/v) including a mixture of POPC/PHPC phospholipids, a peptide mimetic of apoA-I, a mixture of C16-C20 triglycerides, a mixture of cholesterol and one or more steryl esters, and a hydrophobic polymer. The mixture is homogenized for 5 minutes at 10,000-15,000 rpm (Vitris homogenizer model Tempest I.Q.) in order to form a crude emulsion, and then transferred into a high pressure homogenizer. The emulsification is performed at 20,000 psi while recycling the emulsion. The resulting system is transferred into a Rotavap, and the solvent is rapidly removed at 40° C. at reduced pressure (25 mm of Hg). The resulting dispersion is translucent. The dispersion is serially filtered through multiple filters. The size of the filtered formulation is 35-100 nm.

### Microfluidizer Example 3—Lyophilization

The nanobiologic is formed as in either of the above examples. The dispersion is further lyophilized (FTS Systems, Dura-Dry μP, Stone Ridge, N.Y.) for 60 hours. The resulting lyophilization cake is easily reconstitutable to the original dispersion by the addition of sterile water or 0.9% (w/v) sterile saline. The particle size after reconstitution is the same as before lyophilization.

### Prodrug

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of nanobiologic composition of the invention that comprise biohydrolyzable moieties such as biohydrolyzable amides, biohydrolyzable esters, biohydrolyzable ethers, biohydrolyzable carbamates, biohydrolyzable carbonates, biohydrolyzable ureides, and biohydrolyzable phosphate analogues. Other examples of prodrugs include non-biohydrolyzable moieties that nonetheless provide the stability and functionality. Other examples of prodrugs include derivatives of nanobiologic composition of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, N.Y. 1985).

Increasing a drug's compatibility with nanobiologics can be achieved using the strategy described below. A drug is covalently coupled to a hydrophobic moiety, such as cholesterol. If required, a prodrug approach can be achieved via a labile conjugation, resulting in e.g., an enzymatically cleavable prodrug.

Subsequently, the derivatized drug is incorporated into lipid based nanobiologics used for in vivo drug delivery. The main goal of the drug derivatization is to form a drug-conjugate with a higher hydrophobicity as compared to the parent drug. As a result, the retention of the drug-conjugate inside the nanobiologic is enhanced compared to that of the parent drug, thereby resulting in reduced leakage and improved delivery to the target tissue. In case of the prodrug strategy, different type of hydrophobic moieties might give rise to different in vivo cleavage rates, thereby influencing the rate with which the active drug is generated, and thus the overall therapeutic effect of the nanobiologic-drug construct.

Amongst others, lipids, sterols, polymers and aliphatic side-chains can be used as hydrophobic moieties. An optimized derivatization of the mTORi HDL nanobiologic with carbon chains to increase hydrophobicity has been synthesized according to these methods. Additionally, in additional embodiments, the inclusion of triglycerides in HDL create a larger and more miscible hydrophobic core for loading of the active agent, such as the mTOR inhibitor.

Combination with Second Active Agents

Nanobiologic composition can be combined with other pharmacologically active compounds (“second active agents”) in methods and compositions of the invention. It is believed that certain combinations work synergistically in the treatment of particular types of transplantation, atherosclerosis, arthritis, inflammatory bowel disease, and certain diseases and conditions associated with, or characterized by, undesired autoimmune activity. Nanobiologic composition can also work to alleviate adverse effects associated with certain second active agents, and some second active agents can be used to alleviate adverse effects associated with nanobiologic composition.

### Small Molecule Secondary Agents

Small molecule drugs that can be used in combination therapy with the nanobiologics of the present invention include prednisone, prednisolone, methylprednisolone, dezmethasone, betamethasone, acetylsalicylic acid, phenylbutazone, indomethacin, diflunisal, sulfasalazine, acetaminophen, mefenamic acid, meclofenamate, flufenamic acid, ibuprofen, naproxen, fenoprofen, ketoprofen, flurbiprofen, oxaprozin, piroxicam, tenoxicam, saicylate, nimesulide, celecoxib, rofecoxib, valdecoxib, lumiracoxib, parecoxib, etoricoxib, methotrexate, leflunomide, sulfasalazine, azathioprine, cyclophosphamide, antimalarials hydroxychloroquine and chloroquine, d-penicillamine, and cyclosporine.

### Dosing

Dosing will generally be in the range of 5 g to 100 mg/kg body weight of recipient (mammal) per day and more usually in the range of 5 g to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of a nanobiologic which comprises an inhibitor, wherein the inhibitor or a pharmaceutically acceptable salt, solvate, poly-morph, tautomer or prodrug thereof, formulated as nanobiologic using the nanoscale assembly (IMPEPi-NA).

In another preferred embodiment, the inhibitor may include, an mTOR inhibitor (mTORi-NA), a S6K1 inhibitor (S6K1i-NA), Diethyl malonate (DMM), 3BP, 2-DG (DMM-NA) (generally glycolysis inhibiting-Gly-NA), or Camptothecin (Hif-1a), or Tacrolimus+Nanoscale Assembly.

### Combination Therapy

Compounds of the present invention for inhibiting trained immunity, and their salts and solvates, and physiologically functional derivatives thereof, may be employed alone or in combination with other therapeutic agents for the treatment of diseases and conditions. Combination therapy of the nanobiologic with a secondary therapeutic agent may include co-administration with a known immunosuppressant compound. Exemplary immunosuppressants include, but are not limited to, statins; mTOR inhibitors, such as rapamycin or a rapamycin analog; TGF-beta. signaling agents; TGF-beta. receptor agonists; histone deacetylase (HDAC) inhibitors; corticosteroids; inhibitors of mitochondrial function, such as rotenone; P38 inhibitors; NF-kappa beta. inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cycosporine A, siRNAs targeting cytokines or cytokine receptors and the like. Examples of statins include atorvastatin (LIPITOR®, TORVAST®), cerivastatin, fluvastatin (LESCOL®, LESCOL® XL), lovastatin (MEVACOR®, ALTOCOR®, ALTOPREV®), mevastatin (COMPACTIN®), pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®)

### Transplantation

A “transplantable graft” refers to a biological material, such as cells, tissues and organs (in whole or in part) that can be administered to a subject. Transplantable grafts may be autografts, allografts, or xenografts of, for example, a biological material such as an organ, tissue, skin, bone, nerves, tendon, neurons, blood vessels, fat, cornea, pluripotent cells, differentiated cells (obtained or derived in vivo or in vitro), etc. In some embodiments, a transplantable graft is formed, for example, from cartilage, bone, extracellular matrix, or collagen matrices. Transplantable grafts may also be single cells, suspensions of cells and cells in tissues and organs that can be transplanted. Transplantable cells typically have a therapeutic function, for example, a function that is lacking or diminished in a recipient subject. Some non-limiting examples of transplantable cells are islet cells, beta-cells, hepatocytes, hematopoietic stem cells, neuronal stem cells, neurons, glial cells, or myelinating cells. Transplantable cells can be cells that are unmodified, for example, cells obtained from a donor subject and usable in transplantation without any genetic or epigenetic modifications. In other embodiments, transplantable cells can be modified cells, for example, cells obtained from a subject having a genetic defect, in which the genetic defect has been corrected, or cells that are derived from reprogrammed cells, for example, differentiated cells derived from cells obtained from a subject.

“Transplantation” refers to the process of transferring (moving) a transplantable graft into a recipient subject (e.g., from a donor subject, from an in vitro source (e.g., differentiated autologous or heterologous native or induced pluripotent cells)) and/or from one bodily location to another bodily location in the same subject.

In an embodiment, the transplanted tissue is lung tissue, heart tissue, kidney tissue, liver tissue, retinal tissue, corneal tissue, skin tissue, pancreatic tissue, intestinal tissue, genital tissue, ovary tissue, bone tissue, tendon tissue, or vascular tissue.

In an embodiment, the transplanted tissue is transplanted as an intact organ.

As used herein a “recipient subject” is a subject who is to receive, or who has received, a transplanted cell, tissue or organ from another subject.

As used herein a “donor subject” is a subject from whom a cell, tissue or organ to be transplanted is removed before transplantation of that cell, tissue or organ to a recipient subject.

In an embodiment the donor subject is a primate. In a further embodiment the donor subject is a human. In an embodiment the recipient subject is a primate. In an embodiment the recipient subject is a human. In an embodiment both the donor and recipient subjects are human. Accordingly, the subject invention includes the embodiment of xenotransplantation. As used herein “rejection by an immune system” describes the event of hyperacute, acute and/or chronic response of a recipient subject's immune system recognizing a transplanted cell, tissue or organ from a donor as non-self and the consequent immune response.

The term “allogeneic” refers to any material derived from a different animal of the same species as the individual to whom the material is introduced. Two or more individuals are said to be allogeneic to one another when the genes at one or more loci are not identical.

The term “autologous” refers to any material derived from the same individual to whom it is later to be re-introduced into the same individual.

As used herein an “immunosuppressant pharmaceutical” is a pharmaceutically-acceptable drug used to suppress a recipient subject's immune response. A non-limiting example includes rapamycin.

### Pharmaceutical Delivery

As used herein, a “prophylactically effective” amount is an amount of a substance effective to prevent or to delay the onset of a given pathological condition in a subject to which the substance is to be administered. A prophylactically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “therapeutically effective” amount is an amount of a substance effective to treat, ameliorate or lessen a symptom or cause of a given pathological condition in a subject suffering therefrom to which the substance is to be administered.

In one embodiment, the therapeutically or prophylactically effective amount is from about 1 mg of agent/kg subject to about 1 g of agent/kg subject per dosing. In another embodiment, the therapeutically or prophylactically effective amount is from about 10 mg of agent/kg subject to 500 mg of agent/subject. In a further embodiment, the therapeutically or prophylactically effective amount is from about 50 mg of agent/kg subject to 200 mg of agent/kg subject. In a further embodiment, the therapeutically or prophylactically effective amount is about 100 mg of agent/kg subject. In still a further embodiment, the therapeutically or prophylactically effective amount is selected from 50 mg of agent/kg subject, 100 mg of agent/kg subject, 150 mg of agent/kg subject, 200 mg of agent/kg subject, 250 mg of agent/kg subject, 300 mg of agent/kg subject, 400 mg of agent/kg subject and 500 mg of agent/kg subject.

### Methods of Treatment and Prevention

Methods of this invention encompass methods of treating, preventing and/or managing various types of transplantation, atherosclerosis, arthritis, inflammatory bowel disease, and diseases and disorders associated with, or characterized by, undesired autoimmune activity.

As used herein, unless otherwise specified, the term “treating” refers to the administration of a compound of the invention or other additional active agent after the onset of symptoms of the particular disease or disorder.

The phrase “treating” or “treatment” of a state, disorder or condition includes:

preventing or delaying the appearance of clinical symptoms of the state, disorder, or condition developing in a person who may be afflicted with or predisposed to the state, disorder or condition but does not yet experience or display clinical symptoms of the state, disorder or condition; or
inhibiting the state, disorder or condition, i.e., arresting, reducing or delaying the development of the disease or a relapse thereof (in case of maintenance treatment) or at least one clinical symptom, sign, or test, thereof; or
relieving the disease, i.e., causing regression of the state, disorder or condition or at least one of its clinical or sub-clinical symptoms or signs.

As used herein, unless otherwise specified, the term “preventing” refers to the administration prior to the onset of symptoms, particularly to patients at risk of transplantation, atherosclerosis, arthritis, inflammatory bowel disease, and other diseases and disorders associated with, or characterized by, undesired autoimmune activity. The term “prevention” includes the inhibition of a symptom of the particular disease or disorder. Patients with familial history of transplantation, atherosclerosis, arthritis, inflammatory bowel disease, and diseases and disorders associated with, or characterized by, undesired autoimmune activity are preferred candidates for preventive regimens.

As used herein and unless otherwise indicated, the term “managing” encompasses preventing the recurrence of the particular disease or disorder in a patient who had suffered from it, and/or lengthening the time a patient who had suffered from the disease or disorder remains in remission.

In another embodiment, this invention encompasses a method of treating, preventing and/or managing transplantation, atherosclerosis, arthritis, inflammatory bowel disease, which comprises administering an nanoscale particle of the invention, or a pharmaceutically acceptable salt, solvate, hydrate, stereoisomer, clathrate, or prodrug thereof, in conjunction with (e.g. before, during, or after) conventional therapy including, but not limited to, surgery, immunotherapy, biological therapy, radiation therapy, or other non-drug based therapy presently used to treat, prevent or manage transplantation.

Radiolabelling for Pet Imaging of Accumulation of Drug within the Body

In a non-limiting preferred embodiment of the invention, there is provided radiopharmaceutical compositions and methods of radiopharmaceutical imaging an accumulation of a nanobiologic within bone marrow, blood, and/or spleen, of a patient affected by trained immunity, comprising:

administering to said patient a nanobiologic composition in an amount effective to promote a hyper-responsive innate immune response,
wherein the nanobiologic composition comprises (i) a nanoscale assembly, having (ii) an inhibitor drug incorporated in the nanoscale assembly, and (iii) a positron emission tomography (PET) imaging agent incorporated in the nanoscale assembly,
wherein the nanoscale assembly is a multi-component carrier composition comprising: (a) phospholipids, and, (b) apoA-I or a peptide mimetic of apoA-I, and optionally (c) a hydrophobic matrix comprising one or more triglycerides, fatty acid esters, hydrophobic polymers, or sterol esters, or a combination thereof, and optionally (d) cholesterol,
wherein the inhibitor of a metabolic pathway or an epigenetic pathway comprises: a NOD2 receptor inhibitor, an mTOR inhibitor, a ribosomal protein S6 kinase beta-1 (S6K1) inhibitor, an HMG-CoA reductase inhibitor (Statin), a histone H3K27 demethylase inhibitor, a BET bromodomain blockade inhibitor, an inhibitor of histone methyltransferases and acetyltransferases, an inhibitor of DNA methyltransferases and acetyltransferases, an inflammasome inhibitor, a Serine/threonine kinase Akt inhibitor, an Inhibitor of Hypoxia-inducible factor 1-alpha, also known as HIF-1-alpha, and a mixture of one or more thereof,
wherein the PET imaging agent is selected from ⁸⁹Zr, ¹²⁴I, ⁶⁴Cu, ¹⁸F and ⁸⁶Y, and wherein the PET imaging agent is complexed with nanobiologic using a suitable chelating agent to form a stable drug-agent chelate,
wherein said nanobiologic, in an aqueous environment, self-assembles into a nanodisc or nanosphere with size between about 8 nm and 400 nm in diameter,
wherein the nanoscale assembly delivers the stable drug-agent chelate to myeloid cells, myeloid progenitor cells or hematopoietic stem cells in bone marrow, blood and/or spleen of the patient,
and
(ii) performing PET imaging of the patient to visualize biodistribution of the stable drug-agent chelate within the bone marrow, blood, and/or spleen of the patient's body

Further, ex vivo methods may be used to quantify tissue uptake of the ⁸⁹Zr labeled nanoparticles using gamma counting or autoradiography to validate the imaging results. This also provides an novel approach to autoradiography-based histology, which allows the evaluation of the nanomaterial's regional distribution within the tissue of interest by comparing the radioactivity deposition pattern—obtained by autoradiography—with histological and/or immunohistochemical stains on the same or adjacent sections.

Currently, the most commonly used methods to assess nanotherapeutics' in vivo behavior rely on fluorescent dyes. However, these techniques are not quantitative due to autofluorescence, quenching, FRET, and the high sensitivity of fluorophores to the environment (e.g., pH or solvent polarity). The integration of magnetic resonance imaging imaging agents as nanoparticle labels has been trialed, but requires high payloadz and dosing, compromising the integrity of nanoparticle formulations. Nuclear imaging agents do not have these shortcomings, with Zr being especially suited due to its emission of positrons necessary for PET imaging, as well as its relatively long physical half-life (78.4 hours), which allows for longitudinal studies of slow-clearing substances and eliminates the need for a nearby cyclotron.

Our approach provides an excellent way to functionalize nanobiologics using ⁸⁹Zr. DSPE-DFO represents a stable way to anchor the DFO chelator into lipid mono- or bilayers. In addition, as DFO is present on the outside of the nanoparticle platform, the nanoparticles can be labeled after they are formulated. This eliminates the need to perform their formulation under radio-shielded conditions, and reduces the amount of activity that needs to be employed. Lastly, the mild conditions with which DSPE-DFO is incorporated, and ⁸⁹Zr introduced, are compatible with a wide variety of nanoparticle types and formulation methods.

In yet another preferred embodiment of the invention, where further stabilty is desired in the formulation, the invention a lipophilic DFO derivative, named C34-DFO,⁶ that can be incorporated following the same protocol.

In yet a further non-limiting preferred embodiment of the invention, the invention includes radiolabeled protein-coated nanoparticles prepared by first formulating the particles, then functionalizing the protein component with commercially available p-NCS-Bz-DFO, and finally introducing ⁸⁹Zr using our general procedure.

### EXAMPLES

### Transplantation Immunity Results—Examples 1-13

### Example 1—Transplantation Immunity—Donor Allograft Expresses Vimentin and HMGB1 and Promotes Local Training of Macrophages

To decipher macrophage activation pathways that promote allograft immunity, the functional state of macrophages with increased inflammatory cytokine production caused by non-permanent epigenetic reprogramming associated with trained immunity was evaluated. The role for dectin-1 and TLR4 agonists vimentin and the high mobility group box 1 (HMGB1) that may be present under sterile inflammation was shown.

BALB/c (H2d) hearts were transplanted into fully allogeneic C57BL/6 (H2b) recipients as described and data in FIGS. 1-3 indicate that both proteins were upregulated in the donor allograft following organ transplantation. This shows that vimentin and HMGB1 are able to promote training of graft-infiltrating macrophages locally.

To confirm, graft-infiltrating macrophages expressed dectin-1 and TLR4 by flow cytometry are shown in FIG. 4. Absence of dectin-1 and TLR4 expression using deficient recipient mice prevented the accumulation of graft-infiltrating inflammatory Ly6Chi macrophages (FIG. 5). Conversely, dectin-1 or TLR4-deficiency promoted the accumulation of Ly6Clo macrophages in the allograft, which promote allograft tolerance.

Having demonstrated that donor allografts upregulated vimentin and HMGB1, vimentin and HMGB1 were shown to promote macrophage training. Using an established in vitro trained immunity model, in which purified monocytes are exposed to β-glucan followed by re-stimulation with LPS, a similar increase was observed in the production of the pro-inflammatory cytokines TNFα and IL-6 upon vimentin and HMGB1 stimulation (FIG. 6), indicative of these proteins' ability to induce macrophage training. To validate that vimentin and HMGB1 induced local training of graft infiltrating macrophages, these cells were flow sorted from heart allografts and their ability to produce pro-inflammatory cytokines and glycolytic products evaluated. It was shown that dectin-1 or TLR4 deficiency significantly lowered pro-inflammatory TNFα and IL-6 expression and lactate production by graft-infiltrating macrophages after ex vivo LPS stimulation (FIG. 7). In line with the protein expression, absence of dectin-1 or TLR4 prevented H3K4me3 epigenetic changes in the promoter of the pro-inflammatory cytokines TNFα and IL-6 and the glycolytic enzymes hexokinase (HK) and phosphofructokinase (PFKP) in graft-infiltrating macrophages (FIG. 8). Collectively, the data shows that monocyte precursors in the bone marrow (FIG. 34) migrate to the allograft early after transplantation and become trained following vimentin/HMGB1 exposure locally.

### Example 2—Transplantation Immunity—mTORi-HDL Nanoimmunotherapy Prevents Trained Immunity In Vitro

In another preferred aspect of the invention, a nanoimmunotherapy based on high-density lipoprotein (HDL) nanobiologics was developed to target myeloid cells. Since the mammalian target for rapamycin (mTOR) regulates cytokine production (signal 3) through trained immunity, the mTOR inhibitor rapamycin (FIG. 35) was encapsulated in a corona of natural phospholipids and apolipoprotein A-I (apoA-I) isolated from human plasma, to render mTORi-HDL nanobiologics.

The resulting nanobiologics had a drug encapsulation efficiency of 62±11% and a mean hydrodynamic diameter of 12.7±4.4 nm, as determined by high performance liquid chromatography and dynamic light scattering, respectively. Transmission electron microscopy revealed mTORi-HDL to have the discoidal structure (FIGS. 9 and 36; STAR Methods).

### Example 3—Transplantation Immunity—Immunity Model

Using an established in vitro trained immunity model, in which purified human monocytes are exposed to β-glucan, increased cytokine and lactate production upon re-stimulation with LPS was observed. Conversely, β-glucan-trained human monocytes treated with mTORi-HDL during the training period displayed significantly less cytokine and lactate production upon LPS re-stimulation (FIG. 10). This result showed trained immunity to be mTOR-dependent. As the higher cytokine and glycolytic responses may be the result of macrophages' epigenetic reprogramming, trimethylation of the histone H3K4 was assessed, which designates open chromatin (FIG. 11; STAR Methods). mTORi-HDL treatment prevented epigenetic changes at the promoter level of four inflammatory genes associated with trained immunity in human monocytes.

### Example 4—Transplantation Immunity—Biodistribution

The biodistribution and immune cell specificity of fluorescent-dyed (DiO or DiR) or zirconium-89 radiolabeled mTORi-HDL is shown (⁸⁹Zr-mTORi-HDL; FIG. 12; STAR Methods), using a combination of in vivo positron emission tomography with computed tomography (PET-CT) imaging, ex vivo near infrared fluorescence (NIRF) imaging and flow cytometry in C57BL/6 wild-type mice (FIG. 13). The figures show the detection of ⁸⁹Zr-mTORi-HDL accumulation in the kidney, liver and spleen (FIG. 14 and FIGS. 37-38), preferentially associated with myeloid cells, but not with T or B cells (FIG. 15). Importantly, strong mTORi-HDL accumulation in the bone marrow was observed (FIGS. 14-15) and was associated with several myeloid cells and their progenitors (FIG. 16), to facilitate the induction of prolonged therapeutic effects.

### Example 5—Transplantation Immunity—mTORi-HDL Nanoimmunotherapy Prevents Trained Immunity In Vivo

mTORi-HDL treatment was applied to an experimental heart transplant mouse model (FIG. 17) and determined allograft targeting and immune cell specificity as described above. Six days after receiving heterotopic heart transplants, mice were treated with intravenous ⁸⁹Zr-mTORi-HDL. The nanoimmunotherapy was allowed to circulate and distribute for 24 hours before mice were subjected to PET-CT. The figures show marked ⁸⁹Zr-mTORi-HDL presence in the heart allografts (FIGS. 18 and 39; STAR Methods). After mice were sacrificed, the native heart and allograft were collected for ex vivo ⁸⁹Zr quantification. The figures also show radioactivity (25.2±2.4×103 counts/unit area) in the heart allograft (Tx) to be 2.3-fold higher than in native hearts (N) (11.1±1.9×103 count/unit area) (FIG. 19).

### Example 6—Transplantation Immunity—Immune Cell Specificity

Since the nanoimmunotherapy showed favorable organ distribution pattern and heart allograft uptake, immune cell specificity of mTORi-HDL that had been labeled with the fluorescent dye DiO was evaluated. 24 hours after intravenous administration, the heart allograft, as well as blood and spleen, were collected and measured for mTORi-HDL distribution in DC, macrophages, neutrophils and T cells by flow cytometry. The mTORi-HDL cellular preference towards myeloid cells is shown in the figures, with significantly higher uptake by macrophages than either DC or neutrophils in the allograft, blood and spleen (FIGS. 20 and 40-41). T cells exhibited poor mTORi-HDL uptake (FIGS. 42 and 43), which highlights the mTORi-HDL's preferential targeting of myeloid cells.

### Example 7—Transplantation Immunity—Treatment Regimen

A treatment regimen involving three intravenous mTORi-HDL injections at 5 mg/kg rapamycin per dose, at the day of transplantation as well as on postoperative days 2 and 5 was assessed. The myeloid cell compartment in the allograft, blood and spleen of mice receiving either mTORi-HDL treatments or placebo was profiled. In line with the targeting data, the overall numbers of macrophages, neutrophils and DC were significantly lower in the allograft, blood and spleen (FIG. 44) of mTORi-HDL-treated recipients, in comparison with either placebo or mice treated with oral rapamycin (5 mg/kg on postoperative days 0, 2, and 5).

### Example 8—Transplantation Immunity—Macrophage Subsets

mTORi-HDL nanoimmunotherapy's effect on the distribution of two different macrophage subsets (Ly-6Chi and Ly-6Clo), which have distinct immune regulatory properties, is also provided in the figures. Six days after transplantation, untreated recipient mice had increased numbers of inflammatory Ly-6Chi macrophages in the allograft, blood and spleen (FIGS. 21 and 45). By contrast, mTORi-HDL-treated recipients had increased numbers of Ly-6Clo macrophages. The data indicate that while Ly-6Chi macrophages comprised the majority of macrophages during transplant rejection, our mTORi-HDL nanoimmunotherapy promotes the accumulation of Ly-6Clo macrophages. This change was not observed in animals treated with oral rapamycin (FIG. 45).

### Example 9—Transplantation Immunity—Molecular Pathways

Gene Set Enrichment Analysis (GSEA) of mRNA isolated from flow-sorted macrophages from the allografts of animals treated with either placebo or mTORi-HDL was used to illustrate the molecular pathways targeted by the mTORi-HDL nanoimmunotherapy. Gene array results indicated that the trained immunity-related mTOR and glycolysis pathways were negatively regulated by mTORi-HDL (FIGS. 22-23). Macrophages from heart allografts were flow sorted and evaluated to demonstrate their ability to produce inflammatory cytokines (signal 3) and glycolytic products. mTORi-HDL treatment was shown to significantly lower TNFα and IL-6 protein expression and lactate production by graft-infiltrating macrophages after ex vivo LPS stimulation (FIG. 24). In line with the in vitro observations (FIGS. 10 and 11), mTORi-HDL treatment also prevented H3K4me3 epigenetic changes in graft-infiltrating macrophages (FIG. 25; STAR Methods).

### Example 10—Transplantation Immunity—Organ Transplant Acceptance

FIG. 26-33 shows mTORi-HDL nanoimmunotherapy promotes organ transplant acceptance. FIG. 26-33 shows the immunological function of graft-infiltrating macrophages. Ly-6Clo macrophages' suppressive function was measured by their capacity to inhibit in vitro proliferation of carboxyfluorescein diacetate succinimidyl ester (CFSE)-labeled CD8+ T cells. Ly-6Clo macrophages obtained from the allografts of mTORi-HDL-treated recipient mice were observed to inhibit T cell proliferation in vitro (FIG. 26). The same mTORi-HDL-treated allograft Ly-6Clo macrophages expand immunosuppressive Foxp3-expressing regulatory T cells (Treg). In accordance with these data, it was observed that significantly more CD4+CD25+ T cells in the allografts of mTORi-HDL-treated recipients (FIG. 27). These results suggested that mTORi-HDL treatment supports transplantation tolerance by promoting the development of Ly-6Clo regulatory macrophages (Mreg).

### Example 11—Transplantation Immunity—Transplant Recipients

As shown in the Figures, the functional role of Ly-6Clo Mreg in transplant recipients is illustrated using depleted Ly-6Clo Mreg in vivo. Briefly, BALB/c (H2d) donor cardiac allografts were transplanted into C57BL/6 fully allogeneic CD169 diphtheria toxin (DT) receptor (DTR) (H2b) recipient mice treated with mTORi-HDL. Regulatory Ly-6Clo Mreg was depleted by DT administration on the day of transplantation (FIG. 28), which resulted in early graft rejection (12.3±1.8 days) despite mTORi-HDL treatment (FIG. 29). Adoptive transfer of wild-type monocytes restored allograft survival, thereby demonstrating that the nanoimmunotherapy exerts its effects through Mreg (FIG. 29). This was further confirmed using CD11c-DTR mice as transplant recipients, in which administration of DT in these mice depletes CD11c+DC. It showed that graft survival prolongation is independent of CD1c+DC. On the contrary, graft survival in CCR2-deficient recipient mice, with fewer Ly-6Chi circulating monocytes, was not prolonged (FIG. 30). Overall, these experiments demonstrate that macrophages are required for mTORi-HDL nanoimmunotherapy-facilitated organ transplant acceptance.

### Example 12—Transplantation Immunity—Co-Stimulatory Blockade

Activated macrophages produce large amounts of IL-6 and TNFα that promote T cell graft-reactive alloimmunity. The absence of recipient IL-6 and TNFα synergizes with the administration of CD40-CD40L co-stimulatory blockade to induce permanent allograft acceptance. This was shown by concurrent co-stimulatory blockade (signal 2) to augment mTORi-HDL's efficacy. To illustrate, a second nanoimmunotherapy treatment consisting of a CD40-TRAF6 inhibitory HDL (TRAF6i-HDL) was used (FIGS. 47 and 48). The specificity for CD40 signaling inhibition was shown using an agonistic CD40 mAb (clone FGK4.5), which induced rejection in mTORi-HDL treated recipients. TRAF6i-HDL nanobiologic treatment was shown to prevent the detrimental effects of stimulatory CD40 mAb and restored mTORi-HDL-mediated allograft survival (FIG. 31).

### Example 13—Transplantation Immunity—Fully Allogeneic Donor Hearts

Nanoimmunotherapy's ability to prolong graft survival of fully allogeneic donor hearts is shown in the figures. Using the aforementioned three-dose regimen of 5 mg/kg per dose on postoperative days 0, 2, and 5, the mTORi-HDL treatment significantly increased heart allograft survival as compared to placebo, HDL vehicle and oral/intravenous rapamycin treatments (FIGS. 32 and 49). A treatment regimen was subsequently tested by combining mTORi-HDL (signal 3) and TRAF6i-HDL (signal 2) nanobiologics. This mTORi-HDL/TRAF6i-HDL treatment synergistically promoted organ transplant acceptance and resulted in >70% allograft survival 100 days post-transplantation. The combined treatment dramatically outperformed the mTORi-HDL and TRAF6i-HDL monotherapies (FIG. 32) without histopathological evidence for toxicity or chronic allograft vasculopathy (FIGS. 33 and 50).

Collectively, the data showed that HDL-based nanoimmunotherapy prevents macrophage-derived inflammatory cytokine production associated with trained immunity. Further, HDL-based nanoimmunotherapy presented less toxicity than an oral rapamycin resulting in prolonged therapeutic benefits without off-target side effects (FIG. 51).

### Example 14—Transplantation Immunity—Materials and Methods

### Mice

Female C57BL/6J (B6 WT, H-2b) and BALB/c (H-2d) mice were purchased from the Jackson Laboratory. Eight-week-old C57BL/6J (Foxp3tm1Flv/J), CCR2-deficient, and CD11c-DTR mice were purchased from the Jackson Laboratory. C57BL/6J CD169DTR mice were acquired from Masato Tanaka (Kawaguchi, Japan) (Miyake et al., 2007). Animals were enrolled at 8 to 10 weeks of age (body weight, 20-25 g). All experiments were performed with matched 8- to 12-week-old female mice in accordance with protocols approved by the Mount Sinai Animal Care and Utilization Committee.

### Human Samples

Buffy coats from pooled unspecified gender healthy donors were obtained after written informed consent (Sanquin blood bank, Nijmegen, The Netherlands). Gender and age of healthy donors was not collected and is therefore unavailable.

### Method Details

### Vascularized Heart Transplantation

BALB/c hearts were transplanted as fully vascularized heterotopic grafts into C57BL/6 mice as previously described (Corry et al., 1973). Hearts were transplanted into recipients' peritoneal cavities by establishing end-to-side anastomosis between the donor and recipient aortae and end-to-side anastomosis between the donor pulmonary trunk and the recipient inferior vena cava. Cardiac allograft survival was subsequently assessed through daily palpation. Rejection was defined as the complete cessation of cardiac contraction and was confirmed by direct visualization at laparotomy. Graft survival was compared among groups using Kaplan-Meier survival analysis.

### Apolipoprotein A-I (Apoa-I) Isolation

Human apoA-I was isolated from human HDL concentrates (Bioresource Technology) following a previously described procedure (Zamanian-Daryoush et al., 2013). Briefly, a potassium bromide solution (density: 1.20 g/mL) was layered on top of the concentrate and purified HDL was obtained by ultracentrifugation. The purified fraction was added to a chloroform/methanol solution for delipidation. The resulting milky solution was filtered and the apoA-I precipitate was allowed to dry overnight. The protein was renatured in 6 M guanidine hydrochloride, and the resulting solution dialyzed against PBS. Finally, the apoA-I PBS solution was filtered through a 0.22 μm filter and the protein's identity and purity were established by gel electrophoresis and size exclusion chromatography.

### Nanobiologic Synthesis

mTORi-HDL nanoparticles were synthesized using a modified lipid film hydration method. Briefly, 1,2-dimyristoyl-sn-glycero-3-phosphatidylcholine (DMPC), 1-myristoyl-2-hydroxy-sn-glycero-phosphocholine (MHPC) (both purchased from Avanti Polar Lipids) and rapamycin (Selleckchem) were dissolved in a chloroform/methanol (10:1 v/v) mixture at a 3:1:0.5 weight ratio. After evaporating the solvents, human apoA-I in PBS was added to hydrate the lipid film, in a phospholipid to apoA-I 5:1 weight ratio, and left to incubate for 20 minutes in an ice bath. The resulting mixture was homogenized using a probe sonicator in an ice bath for 15 minutes to yield mTORi-HDL nanoparticles. mTORi-HDL was washed and concentrated by centrifugal filtration using 10 kDa molecular weight cut-off (MWCO) filter tubes. Aggregates were removed using centrifugation and filtration (0.22 μm). For the therapeutic studies, animals received oral doses or intravenous tail injections (for mTORi-HDL or intravenous Ra) at a rapamycin dose of 5 mg/kg on the day of transplantation, as well as days two and five post-transplantation.

HDL nanobiologics size and surface charge was determined by dynamic light scattering (DLS) and Z-potential measurements. The final composition after purification was determined by standard protein and phospholipid quantification methods (bicinchoninic acid assay and malachite green phosphate assay), whereas drug concentration was established by HPLC against a calibration curve of the reference compound. A variability of ±15% between batches was considered acceptable.

Radiolabeling mTORi-HDL Nanoparticles

mTORi-HDL was radiolabeled with ⁸⁹Zr according to previously described procedures (Perez-Medina et al., 2015). Briefly, ready-to-label mTORi-HDL was obtained by adding 1 mol % of the phospholipid chelator DSPE-DFO at the expense of DMPC in the initial formulation. Radiolabeling with ⁸⁹Zr was achieved by reacting the DFO-bearing nanoparticles with ⁸⁹Zr-oxalate in PBS (pH=7.1) at 37° C. for one hour. ⁸⁹Zr-mTORi-HDL was isolated by centrifugal filtration using 10 kDa MWCO tubes. The radiochemical yield was 75±2% (n=2).

### Micro-PET/CT Imaging and Biodistribution Studies

Mice (n=6; 3 with heart transplants [weight: 18.8±1.0 g]) were injected with a single ⁸⁹Zr-mTORi-HDL (0.17±0.01 mCi, ˜0.25 mg apoA-I) dose in 0.2 mL PBS solution via their lateral tail vein six days post graft transplantation. 24 hours later, animals were anesthetized with isoflurane (Baxter Healthcare, Deerfield, USA)/oxygen gas mixture (2% for induction, 1% for maintenance), and a scan was then performed using an Inveon PET/CT system (Siemens Healthcare Global, Erlangen, Germany). Whole body PET static scans, recording a minimum of 30 million coincident events, were performed for 15 minutes. The energy and coincidence timing windows were 350-700 keV and 6 ns, respectively. The image data were normalized to correct for PET response non-uniformity, dead-time count losses, positron branching ratio and physical decay to the time of injection, but no attenuation, scatter or partial-volume averaging correction was applied. The counting rates in the reconstructed images were converted to activity concentrations (percentage injected dose [% ID] per gram of tissue) using a system calibration factor derived from imaging a mouse-sized water-equivalent phantom containing ⁸⁹Zr. Images were analyzed using ASIPro VMTM software (Concorde Microsystems, Knoxville, USA) and Inveon Research Workplace (Siemens Healthcare Global, Erlangen, Germany) software. Whole body standard low magnification CT scans were performed with the X-ray tube setup at a voltage of 80 kV and current of 500 μA. The CT scan was acquired using 120 rotational steps for a total of 220 degrees to yield an estimated scan time of 120 s with an exposure of 145 ms per frame. Immediately after the PET/CT scan, animals were sacrificed and tissues of interest—kidney, heart, liver, spleen, blood, bone, skin and muscle—were collected, weighed and counted on a Wizard2 2480 automatic gamma counter (Perkin Elmer, Waltham, USA) to determine radioactivity content. The values were decay-corrected and converted to percentage of injected dose per gram (% ID/g). To determine radioactivity distribution within the transplanted hearts, the native and grafted specimens were placed in a film cassette against a phosphorimaging plate (BASMS-2325, Fujifilm, Valhalla, USA) for 4 hours at −20° C. The plate was read at a pixel resolution of 25 m with a Typhoon 7000IP plate reader (GE Healthcare, Pittsburgh, USA). The images were analyzed using ImageJ software.

### Immunofluorescence Microscopy

Transplanted hearts were harvested, subdivided, frozen directly in Tissue-Tek OCT (Sakura), and stored at −80° C. in preparation for immunological studies. Sections of 8 μm were cut using a Leica 1900CM cryomicrotome mounted on polylysine-coated slides, and fixed in acetone (at −20C degrees for 20 minutes) and then incubated with blocking buffer containing 1% BSA and 5% goat or rabbit serum. The slides were then incubated overnight at 4C with 1/100 rat anti-muse dectin1 (clone 2A11) or rabbit anti-mouse vimentin (clone EPR3776) from Abcam. After overnight incubation the slides were washed in PBS and then incubated with conjugated goat monoclonal anti-rabbit Cy-3 (1/800) or a goat monoclonal anti-rat Cy-2 (1/500) purchased from Jackson Immunoresearch. All slides were mounted with Vectashield with Dapi (Vector Laboratories) to preserve fluorescence. Images were acquired with a Leica DMRA2 fluorescence microscope (Wetzlar) and a digital Hamamatsu charge-coupled device camera. Separate green, red, and blue images were collected and analyzed with ImageJ software (NIH).

### Isolation of Graft-Infiltrating Leukocytes

Mouse hearts were rinsed in situ with HBSS with 1% heparin. Explanted hearts were cut into small pieces and digested for 40 minutes at 37° C. with 400 U/ml collagenase A (Sigma-Aldrich), 10 mM HEPES (Cellgro) and 0.01% DNase I (MP Biomedicals) in HBSS (Cellgro). Digested suspensions were passed through a nylon mesh and centrifuged, and the cell pellet was re-suspended in complete HBSS, stained and analyzed by flow cytometry (BD LSR-II; BD Biosciences).

### Flow Cytometry and Cell Sorting

For myeloid cell staining, fluorochrome-conjugated mAbs specific to mouse CD45 (clone 30-F11), CD11b (clone M1/70), CD11c (clone N418), F4/80 (clone CI:A3.1), Ly-6C (clone HK1.4) and corresponding isotype controls were purchased from eBioscience. Ly-6G (clone 1A8) mAb was purchased from Biolegend. For T-cell staining, antibodies against CD3 (clone 2C11), CD4 (clone GK1.5), CD8 (clone 53-6.7), and CD25 (clone PC61.5) were purchased from eBioscience. The absolute cell counting was performed using countbright beads (Invitrogen). For progenitor, myeloid and lymphoid cell staining in the bone marrow, spleen, kidney and liver, fluorochrome-conjugated mAbs specific to mouse B220/CD45R (clone RA3-6B2), CD34 (clone RAM34), CD16/32 (clone 93), CD90 (clone 53-2.1), CD19 (clone 1D3), CD115 (clone AFS98) and CD135 (clone A2F10) from eBioscience; CD49b (clone DX5), MHCII (clone M5/114.15.2) and Sca-1 (clone D7) were purchased from Biolegend; CD64 (clone X54-5/7.1), CD117 (clone 2B8), and CD172a (clone P84) were purchased from BD Biosciences. Flow cytometric analysis was performed on LSR II (BD Biosciences) and analyzed with FlowJo software (Tree Star, Inc.). Results are expressed as percentage of cells staining or cells counting (cells per milliliter) above background. To purify graft-infiltrating myeloid cells, donor heart single cell suspensions were sorted with an InFlux cell sorter (BD) to achieve >96% purity at the Flow Cytometry Shared Resource Facility at Icahn School of Medicine at Mount Sinai.

### Human Monocyte Trained Immunity Experiments

Human monocytes were isolated and trained as previously described. PBMC isolation was performed by dilution of blood in pyrogen-free PBS and differential density centrifugation over Ficoll-Paque (GE Healthcare, UK). Subsequently, monocyte isolation was performed by hyper-osmotic density gradient centrifugation over Percoll (Sigma). Monocytes (1×107) were plated to 10 cm Petri dishes (Greiner) in 10 ml medium volumes and incubated with either culture medium only as a negative control or 5 g/ml of β-glucan with or without mTORi-HDL (1 μg/ml) for 24 hours (in 10% pooled human serum). At day six, cells were detached from the plate, and 1×105 macrophages were reseeded in 96-well flat bottom plates to be re-stimulated for 24 hours with 200 μl of either RPMI or Escherichia coli LPS (serotype 055:B5, Sigma-Aldrich, 10 ng/ml), after which supernatants were collected and stored at −20° C. Cytokine production was determined in supernatants using commercial ELISA kits for TNFα and IL-6 (R&D systems) following the instructions of the manufacturer. The remaining cells were fixed in 1% methanol-free formaldehyde and sonicated. Immunoprecipitation was performed using an antibody against H3K4me3 (Diagenode, Seraing, Belgium). DNA was isolated with a MinElute PCR purification kit (Quiagen) and was further processed for qPCR analysis using the SYBR green method. Samples were analyzed by a comparative Ct method according to the manufacturer's instructions.

### Mouse Monocyte Trained Immunity Experiments

Bone marrow monocytes were isolated using a monocyte isolation kit (Miltenyi). Monocytic precursors (1×106/well in a 48-well plate) were differentiated in vitro with 10 ng/ml of recombinant murine GM-CSF (peprotech) for 6 days. On day 6, either 10 g/ml of β-glucan (Sigma) or 100 μg/ml of vimentin (R&D systems) was added to the cultures for 24 h. After 3 days of resting, macrophages were restimulated with either 10 ng/ml of LPS (Sigma) or 20 μg/ml of HMGB1 (R&D systems) for 24 h. Cytokine production was determined in supernatants using commercial ELISA kits for TNFα and IL-6 (R&D systems) while the remaining cells were used in chromatin immunoprecipitation (ChIP) assays.

### Mouse Chromatin Immunoprecipitation (Chip)

In vitro bone marrow derived trained macrophages or graft-infiltrating macrophages were used in this assay. The following antibodies were used: anti-H3K4me3 (39159; Active Motif), and anti-IgG (ab171870; Abcam). For experiments with ChIP followed by qPCR, crosslinking was performed for 10 min. For sonication, we used a refrigerated Bioruptor (Diagenode), which we optimized to generate DNA fragments of approximately 200-1,000 base pair (bp). Lysates were pre-cleared for two hours using the appropriate isotype-matched control antibody (rabbit IgG; Abeam). The specific antibodies were coupled with magnetic beads (Dynabeads® M-280 Sheep Anti-Rabbit IgG; ThermoFisher Scientific) overnight at 4° C. Antibody-bound beads and chromatin were then immunoprecipitated overnight at 4° C. with rotation. After washing, reverse crosslinking was carried out overnight at 65° C. After digestion with RNase and proteinase K (Roche), DNA was isolated with a MinElute kit (Qiagen) and used for downstream applications. qPCR was performed using the iQ SYBR Green Supermix (Bio-Rad) according to manufacturer's instructions. Primers were designed using the Primer3 online tool; cross-compared to a visualized murine mm10 genome on the Integrated Genomics Viewer (IGV; Broad).

### Suppression Assay

Spleens of C57BL/6 (H-2b) mice were gently dissociated into single-cell suspensions, and red blood cells were removed using hypotonic ACK lysis buffer. Splenocytes were labeled with CFSE at 5 μM concentration (using molecular probes from Invitrogen) followed by staining with anti-CD8 mAb for 30 minutes on ice. Responder CFSE+CD8+ T-cells were sorted using FACS Aria II (BD Biosciences) with >98% purity. CFSE+CD8+ T-cells were used together with anti-CD3/CD28 microbeads as stimulators. Stimulated CFSE+CD8+ T-cells were cultured with graft-infiltrating Ly-6Clo macrophages, mTORi-HDL or placebo for 72 hours at 37° C. in a 5% C02 incubator. T-cell proliferation was measured by flow cytometric analysis of CFSE dilution on CD8+ T-cells.

### Treg Expansion Assay

Spleens of C57BL/6-Foxp3tm1Flv/J (H-2b) mice were gently dissociated into single-cell suspensions, and red blood cells were removed using hypotonic ACK lysis buffer.

Splenocytes were stained with anti-CD4 mAb for 30 minutes on ice. Responder CD4+ were sorted using FACS Aria II (BD Biosciences) with a purity of >98%. CD4+ T-cells were used together with anti-CD3/CD28 microbeads as stimulators. Stimulated CD4+ T-cells were cultured with graft-infiltrating Ly-6Clo macrophages, mTORi-HDL or placebo for 72 hours at 37° C. in a 5% C02 incubator. Treg expansion was measured by flow cytometric analysis of Foxp3-RFP on CD4+ T-cells.

### Enzyme-Linked Immunosorbent Assay (ELISA)

Bone marrow derived macrophages were trained as above. Graft-infiltrating macrophages were isolated as above. TNF-α and IL-6 cytokines produced by trained macrophages in vitro and by graft-infiltrating macrophages was assessed by ELISA (R&D Systems) according to the manufacturer protocol.

### Microarray Analysis

Graft-infiltrating recipient Ly-6Clo macrophages were sorted from mTORi-HDL-treated and placebo-rejecting recipients at day six after transplantation. Cells were sorted twice with a FACS Aria II sorter (BD Biosciences) to achieve >98% purity. Microarray analysis of sorted cells was performed with a total of six Affymetrix Mouse Exon GeneChip 2.0 arrays (Thermo Fisher Scientific) and samples of interest were run in triplicate. Raw CEL file data was normalized using Affymetrix Expression Console Software. Gene expression was filtered based on IQR (0.25) filter using gene filter package. The log 2 normalized and filtered data (adjusted P<0.05) were used for further analysis. Gene signature comparisons were performed between intra-graft Ly6Clo macrophages from mTORi-HDL- and placebo-treated recipients. GSEA was performed using GSEA version 17 from Gene pattern version 3.9.6. Parameters used for the analysis were as follows. Gene sets c2.cp.biocarta.v5.1.symbols.gmt; c2.cp.kegg.v5.1.symbols.gmt; c2.cp.reactome.v5.1.symbols.gmt; c6.all.v5.1.symbols.gmt (Oncogenic Signatures); c7.all.v5.1.symbols.gmt (Immunologic signatures) and h.all.v5.1.symbols.gmt (Hallmarks) were used for running GSEA. To select the significant pathways from each gene set result, fdr q-value of 0.25 was set as cutoff. Only genes that contributed to core enrichment were considered.

### In Vivo Macrophage Depletion

To deplete CD169-expressing Ly-6Clo macrophages, heterozygous CD169-DTR recipients were injected intraperitoneally with 10 ng/g body weight of DT (Sigma-Aldrich) 24, 48 and 72 hours after transplantation.

### Quantification and Statistical Analysis

Statistical analyses Results are expressed as mean±SEM. Statistical comparisons between two groups were evaluated using the Mann-Whitney test or the Wilcoxon signed-rank test for paired measurements. Comparisons among three or more groups were analyzed using the Kruskal-Wallis test followed by Dunn's multiple comparisons test. Kaplan-Meier curves were plotted for allograft survival analysis, and differences between the groups were evaluated using a log-rank test. A value of P≤0.05 was considered statistically significant. GraphPad Prism 7 was used for statistical analysis.

### DATA AND SOFTWARE Availability

The microarray data discussed in this publication have been deposited at NCBI and are accessible through GEO Series accession number GSE119370:

https://urldefense.proofpoint.com/v2/url?u=https-3A__www.ncbi.nlm.nih.gov_geo_query_acc.cgi-3Facc-3DGSE119370&d=DwIEAg&c=shNJtf5dKgNcPZ6Yh64b-A&r=UQzd7yXCG-7V6o6EdZSeY_KvCshJgQzt0LAtZPqCh9Q&m=cuA3YUXFJvxExRDD8AweBNKmcjdYX oyMojyj9IZeQf8&s=f1i6P2_K57m-i40hkuoOxGuMsZH_IKcvtAi3C-9QfmQ&e=

### Atherosclerosis Results—Examples 15-17

### Example 15—mTORi-HDL and the Targeting of Monocytes, Macrophages

Referring to the FIGS. 52-61, In addition to the role of monocytes and macrophages, other cell types, including T cells, endothelial cells and smooth muscle cells, play pivotal roles in the atherosclerosis pathogenesis. As mTOR signaling is universally relevant to cells, systemic mTOR inhibition will affect all cell types involved in atherogenesis. We investigated the effect of inhibiting the mTOR pathway in specifically monocytes and macrophages. To achieve this, we developed an HDL-based nanobiologic that facilitates drug delivery to monocytes and macrophages with high targeting efficiency.

mTORi-HDL was constructed from human apolipoprotein A-I(apoA-I) and the phospholipids 1-myristoyl-2-hydroxy-sn-glycero-phosphocholine (MHPC) and 1,2-dimy-ristoyl-sn-glycero-3-phosphatidylcholine (DMPC), in which the mTOR inhibitor rapamycin was incorporated (FIG. 52). mTORi-HDL measured 23 nm±9 nm (PDI=0.3) as determined by dynamic light scattering. mTORi-HDL variants, incorporating fluorescent dyes (DiO or DiR) were synthesized to enable their detection by fluorescence techniques. Ex vivo near infrared fluorescence (NIRF) imaging performed 24 hours after intravenous administration showed that DiR-labeled mTORi-HDL primarily accumulates in the liver, spleen and kidneys of Apoe−/− mice. High DiR uptake was observed in the aortic sinus area (FIG. 53), which is the preferential site of plaque development in this mouse model. Cellular specificity was evaluated by flow cytometry. For this purpose, DiO-labeled mTORi-HDL was formulated and intravenously injected. We observed DiO-labeled mTORi-HDL to be taken up by 91% of the macrophages and 93% of the Ly6Chi monocytes present in the aorta. Additionally, 50% of the dendritic cells and 73% of the neutrophils were found to contain mTORi-HDL nanobiologics (FIG. 54). Marginal to neglectable mTORi-HDL uptake was observed in non-myeloid (Lin+) cells. These results mirror our findings in blood, spleen and bone marrow, indicating that cells of the myeloid lineage, in particular Ly6Chi monocytes and macrophages, show high uptake of mTORi-HDL.

### Example 16—mTORi-HDL Reduces Plaque Inflammation

To evaluate the effect of mTORi-HDL on plaque inflammation we used 20-week old Apoe−/− mice that had been fed a high-cholesterol diet for 12 weeks to develop atherosclerotic lesions.

While they remained on a high-cholesterol diet, all mice were treated during one week with four intravenous injections of PBS (control, n=7) or mTORi-HDL (containing 5 mg/kg rapamycin, n=10). Mice were euthanized 24 hours after the final infusion. Quantitative histologic analysis of plaque in the aortic sinus area showed no difference in plaque size or collagen content (FIG. 55) as compared to controls. We did observe a 33% (P=0.02) reduction in plaque macrophage content. The Mac3 to collagen ratio in the plaque was decreased by 35% (P=0.004) indicating a more stable plaque phenotype in the mTORi-HDL group (FIG. 55).

Next, we performed fluorescence molecular tomography with computed tomography (FMT-CT) imaging to visualize protease activity in the aortic root area. We used the same mouse model and treatment regimen as described above. Control mice (n=8) and mTORi-HDL treated Apoe−/− mice (n=10) received a single injection of an activatable pan-cathepsin protease sensor 24 hours before imaging. The protease sensor is taken up by activated macrophages and cleaved in the endolysosome, yielding fluorescence as a function of enzyme activity. mTORi-HDL reduced protease activity by 30% (P=0.03, FIG. 58). Together these data provided clear evidence that inhibition of the mTOR signaling pathway in monocytes and macrophages resulted in a rapid reduction of inflammatory activity in atherosclerosis. This incentivized us to unravel the mechanism by which this occurs.

### Example 17—S6K1i-HDL and Targeting of Plaque Monocytes and Macrophages

In the pursuit of understanding the mechanism by which the mTOR signaling pathway controls monocyte and macrophage dynamics in atherosclerosis we focused on the mTOR-S6K1 (S6K1: ribosomal protein S6 kinase beta-1) signaling axis. S6K1 signaling is known to regulate fundamental cellular processes, including transcription, translation, cell growth and cell metabolism, but little is known about its role in regulating innate immune responses in atherosclerosis. For this purpose, we constructed an HDL nanobiologic containing PF-4708671 (S6K1i-HDL), a specific inhibitor of S6K1 (FIG. 59). This nanobiologic was constructed from human apolipoprotein A-I (apoA-J) and the phospholipids 1-myristoyl-2-hydroxy-sn-glycero-phosphocholine (MHPC) and 1,2-dimyristoyl-sn-glycero-3-phosphatidylcholine (DMPC), in which PF-4708671 was incorporated (FIG. 59). S6K1i-HDL measured 34 nm±10 nm (PDI=0.3) as determined by dynamic light scattering. Ex vivo near infrared fluorescence (NIRF) imaging performed 24 hours after infusion into Apoe−/− mice showed that DiR-labeled S6K1i-HDL primarily accumulated in the liver, spleen and kidneys (FIG. 60). In addition, high DiR uptake was observed in the aortic sinus area (FIG. 60), very similar to what we found for mTORi-HDL. Cellular specificity was analyzed by flow cytometry of whole aortas using DiO-labeled S6K1i-HDL (FIG. 61). The percentages of DiO positive cells were 87% for macrophages, 84% for Ly6Chi monocytes, 64% for dendritic cells and 71% for neutrophils (FIG. 61). Uptake in non-myeloid (Lin+) cells was negligible. These results showed that nanobiologic's properties are independent of the therapeutic payload, which enables us to specifically study mTOR and S6K1 inhibition in atherosclerosis. One week of S6K1i-HDL treatment showed a similar trend in the reduction of plaque inflammation as compared to mTORi-HDL (FIG. 62).

Next, in vitro experiments were performed in human adherent monocytes in which trained immunity was induced by oxLDL as described previously (Bekkering et al., 2018). We investigated if mTORi-HDL and S6K1i-HDL nanobiologic treatment inhibited oxLDL-induced trained immunity. Indeed, we found diminished cytokine production upon TLR-4 and TLR-2 mediated re-stimulation with lipopolysaccharide LPS (FIG. 63).

### Example 18—Atherosclerosis Summary and Discussion

Monocytes and macrophages constitute a critical component of our host defense mechanism. Upon recognition of foreign pathogens, these phagocytic cells become activated and mount an inflammatory response to resolve the infection. Sterile substances can also be perceived as danger signals and incite an inflammatory response. This may be appropriate in some cases, but can also be maladaptive, such as in atherosclerosis.

Oxidized low-density lipoprotein cholesterol (oxLDL) and cholesterol crystals are the primary stimuli for the pathogenic innate immune response in atherosclerosis. OxLDL induces transcriptional reprogramming of granulocyte-monocyte progenitor cells, which stimulates pro-inflammatory monocyte production and release from the bone marrow. This results in increased recruitment of inflammatory monocytes to plaques where they differentiate into macrophages. Furthermore and for an important part, plaque inflammation is sustained by local proliferation of macrophages.

OxLDL and cholesterol crystals are also involved in the inflammatory activation of macrophages. OxLDL cholesterol can prime macrophages via activation of a signaling complex formed by a heterodimer of Toll-like receptor 4 (TLR4) and TLR6 together with the scavenger receptor class B member 1 (SRB1) that activates nuclear factor-κB (NF-κB).

Cholesterol crystals induce NLRP3 inflammasome activation by phagolysosomal damage in the macrophages.

Another mechanism by which cholesterol fuels ongoing innate immune cell activation in atherosclerosis is “trained immunity”. Trained immunity, also known as innate immune memory, entices a non-specific immunological memory build-up via epigenetic modifications. This process can be provoked by oxLDL and results in a macrophage phenotype that is characterized by a long-lasting pro-inflammatory response. The oxLDL-induced trained immunity is mediated through NLRP3 inflammasome activation. Thus trained immunity is involved in sustaining inflammatory activity in atherosclerosis. Epigenetic reprogramming of myeloid cells that occurs in trained immunity is associated with marked alterations in cell metabolism. A metabolic shift to aerobic glycolysis induces trained immunity. Not only glucose metabolism but also other metabolic pathways are involved, among which are glutaminolysis and the cholesterol synthesis pathway. Interestingly, the induction of trained immunity by any of these metabolic pathways depends on the activation of the mechanistic target of rapamycin (mTOR), and therefore is a compelling target to prevent trained immunity. The mTOR signaling pathway plays a crucial role in innate immune cell function by acting as an integrative sensor of cellular nutrient status and metabolically coordinating the inflammatory activity of macrophages.

The effect of blocking the mTOR signaling pathway in atherosclerotic monocytes and macrophages was investigated in apolipoprotein E-deficient (Apoe−/−) mice, with the focus on the mTOR-S6K1 axis. To achieve inhibition specifically in myeloid cells, we intravenously administered two different high density-lipoprotein (HDL) nanobiologics that incorporated an mTOR or S6K1 inhibitor, respectively. We observed rapidly reduced plaque inflammation through a combination of diminished macrophage proliferation and inflammatory activity.

The mTOR signaling network is fundamental for balancing anabolism and catabolism in response to the nutritional status in all eukaryotic cells. It plays a dominant role in regulating cellular activity, growth and division. In the present invention, we provide evidence of a mechanistic framework in which mTOR and S6K1 signaling dictates proliferation as well as the inflammatory activity of mononuclear phagocytes in atherosclerosis, both energetically demanding processes.

As claimed and disclosed, we show that cell-specific inhibition of mTOR and S6K1, accomplished by the use of HDL nanobiologics, rapidly suppresses plaque inflammation. We observed this to be the result of diminished local proliferation and a suppressed inflammatory state of macrophages. Transcriptomic analyses of monocytes and macrophages isolated from plaques revealed the key cellular processes that were affected by mTOR and S6K1 inhibition.

These included processes related to cell growth and proliferation, metabolism, and phagocytic function.

Tissue macrophages can be self-maintained by local proliferation. This self-renewing capacity is largely responsible for the expansion of macrophage numbers in advanced plaques. The data in the present invention show that the pharmacologic inhibition of macrophage proliferation, by blocking mTOR and S6K1 signaling, caused prompt reduction of plaque inflammation.

Transcriptomic analyses revealed altered expression of genes related to transcription and translation as well as pathways regulating cell growth and division. Our findings resemble observations made in alternatively activated macrophages. In a mouse model of helminth-induced infection, in which macrophage activation is predominantly induced by interleukin 4 (IL-4), massive local proliferation of macrophages was observed. It was subsequently shown that the IL-4 receptor targets the phosphatidylinositide 3-kinase (PI3K)—Akt signaling pathway which is responsible for the IL-4 induced proliferation. As the PI3K-Akt pathway directly regulates mTOR activation, mTOR was likely to be involved in mediating these effects.

In addition to the effects on proliferation, we also observed that mTORi-HDL and S6K1i-HDL avert myeloid cells from mounting an innate immune memory response. Trained immunity's dependence on the activation of mTOR has been firmly established previously, but our data reveal this also holds true for S6K1 signaling. However, it is interesting to note that S6K1 is not merely a downstream target of mTOR, as this ribosomal protein is capable of inhibiting the phosphorylation of insulin receptor substrate 1 (IRS1). S6K1 thereby suppresses insulin-like growth factor 1 receptor (IGFR) and phosphatidylinositide 3-kinase (PI3K)—Akt signaling, which is upstream in the regulation of mTOR.

The epigenetic reprogramming that occurs in trained immunity goes hand in hand with marked alterations in cell metabolism. In vitro, trained monocytes switch to aerobic glycolysis, probably to prepare them for the metabolic requirement upon reactivation. Metabolic shift influences epigenetic processes and it is clear that metabolites such as acetyl coenzyme A, succinate and α-ketoglutarate can directly affect histone acetylation and methylation. In this context it is interesting that we observed a marked downregulated of oxidative phosphorylation. This is likely to force macrophages into a state of low ATP production, since mTOR-S6K1 inhibition is also known to suppress glycolysis. This low energetic state will negatively impact the ability of macrophages to orchestrate an inflammatory response. How this metabolic reprogramming affects trained immunity was not investigated here and is outside of the scope of the current study.

Atherosclerosis is a lipid-driven inflammatory disease that entices a complex immunologic response, and macrophages are considered the main protagonist. The data we present in this study provide novel insights in the pathogenesis of this disease, by showing that mTOR signaling underlies the chronic maladaptive inflammatory response of macrophages. Both the inflammatory activation in the form of trained immunity and macrophage proliferation were shown to be under the auspices of the mTOR signaling network. These novel mechanistic insights yield new therapeutic opportunities to mitigate the dysfunctional innate immune response in atherosclerosis.

### Example 19—Atherosclerosis Materials and Methods

### Mice

Female Apoe−/− mice (B6.129P2-Apoetm1Unc) were used for this study. Animal care and procedures were based on an approved institutional protocol from Icahn School of Medicine at Mount Sinai. Eight-week-old Apoe−/− mice were purchased from The Jackson Laboratory.

All mice were fed a high-cholesterol diet (0.2% weight cholesterol; 15.2% kcal protein, 42.7% kcal carbohydrate, 42.0% kcal fat; Harlan TD. 88137) for 12 weeks. Littermates were randomly assigned to treatment groups.

In vitro experiments were performed on either the RAW264.7 cell line or bone marrow derived macrophages (BMDMs). RAW264.7 cells were cultured in T75 cm2 Flasks (Falcon), in high glucose Dulbecco's modified Eagle's medium (DMEM) (Gibco Life Technologies). BMDMs were cultured in cell culture dishes, in Roswell Park Memorial Institute medium (RPMI) with addition of 15% L929-cell conditioned medium. All cells were incubated at 37° C. in a 5% C02 atmosphere.

### Human Subjects

For in vitro studies on human monocytes, buffy coats from healthy donors were obtained after written informed consent (Sanquin blood bank, Nijmegen, The Netherlands). For histologic analysis, human atherosclerotic plaque samples were obtained from four patients. All four patients had an indication for carotid endarterectomy. Gender of the included subjects for both studies is known, although gender association cannot be analyzed due to small group sizes. Subject allocation to groups is not applicable.

### Synthesis of Nanobiologics

rHDL nanobiologic formulations were synthesized as shown herein. For mTORi-HDL, the mTORC1-complex inhibitor rapamycin (3 mg, 3.3 μmol), was combined with 1-myristoyl-2-hydroxy-sn-glycero-phosphocholine (MHPC) (6 mg, 12.8 μmol) and 1,2-dimyristoyl-sn-glycero-3-phosphocholine (DMPC) (18 mg, 26.6 μmol) (Avanti Polar Lipids). For S6K1i-HDL, the S6K1 inhibitor PF-4708671 (1.5 mg, 4.6 μmol) was combined with 1-palmitoyl-2-oleoyl-sn-glycero-3-phosphocholine (POPC) (18 mg, 23.7 μmol) and 1-palmitoyl-2-hydroxy-sn-glycero-3-phosphocholine (PHPC) (6 mg, 12.1 μmol). The compounds and lipids were dissolved in methanol and chloroform, mixed, and then dried in a vacuum, yielding a thin lipid film. A PBS solution of human apolipoprotein A1 (apoA-I) (4.8 mg in 5 ml) was added to the lipid film. The mixture was incubated in an ice-cold sonication bath for 15-30 minutes. Subsequently, the solution was sonicated using a tip sonicator at 0° C. for 20 minutes to form rHDL based nanobiologics. The obtained solution was concentrated by centrifugal filtration using a 100 MWCO Vivaspin tube at 3000 rpm to obtain a volume of −1 ml. PBS (5 ml) was added and the solution was concentrated to −1 ml. Again, PBS (5 ml) was added and the solution was concentrated to −1 ml. The remaining solution was filtered through a 0.22 μm PES syringe filter to obtain the final nanobiologic solution. For targeting and biodistribution experiments, analogs of mTORi-HDL and S6K1i-HDL were prepared through incorporation of the fluorescent dyes DiR or DiO (Invitrogen).

### Nanobiologic Treatment

Twenty-week-old Apoe−/− received either PBS, empty rHDL nanobiologics, mTORi-HDL (mTORi at 5 mg/kg), or S6K1i-HDL (S6K1i at 5 mg/kg) through lateral tail vein injections. Mice were treated with 4 injections over 7 days, while being kept on a high-cholesterol diet. For the targeting and biodistribution experiments, mice received a single intravenous injection. All animals were euthanized 24 hours after the last injection.

### Fluorescence Molecular Tomography/X-Ray Computed Tomography

After nanobiologic treatment, mice were injected with 5 nanomoles of pan-cathepsin protease sensor (ProSense 680, PerkinElmer, Cat no. NEV10003). Twenty-four hours later, animals were placed in a custom build cartridge and sedated during imaging with continuous isoflurane administration as described previously (ref). Animals were first scanned using a high-resolution CT scanner (Inveon PET-CT, Siemens), with a continuous infusion of CT-contrast agent (isovue-370, Bracco Diagnostics) at a rate of 55 μL/min through a tail vein catheter. Animals were subsequently scanned using an FMT scanner (PerkinElmer) in the same cartridge. The CT X-ray source with an exposure time of 370-400 ms, was operated at 80 kVp and 500 mA. Contrast-enhanced high-resolution CT images were used to localize the aortic root, which was used to guide the placement of the volume of interest for the quantitative FMT protease activity map. Image fusion relied on fiducial markers. Image fusion and analysis was performed using OsiriX v.6.5.2 (The Osirix Foundation, Geneva).

### Near Infrared Fluorescence Imaging

Mice received a single intravenous injection with DiR (0.5 mg/kg) labeled mTORi-HDL (5 mg/kg) or S6K1i-HDL (5 mg/kg). Liver, spleen, lung, kidneys, heart and muscle tissue were collected for NIRF imaging. Fluorescent images were acquired using an IVIS 200 system (Xenogen), with a 2 second exposure time, using a 745 nm excitation filter and an 820 nm emission filter. ROIs were drawn on each tissue with software provided by the vendor, after which quantitative analyses were performed using the average radiant efficiency within these ROIs.

### Preparation of Single Cell Suspensions

Blood was collected by cardiac puncture and mice were subsequently perfused with 20 mL cold PBS. Spleen and femurs were harvested. The aorta, from aortic root to the iliac bifurcation, was gently cleaned of fat and collected. The aorta was digested using an enzymatic digestion solution containing liberase TH (4 U/ml) (Roche), deoxyribonuclease (DNase) I (40 U/ml) (Sigma-Aldrich), and hyaluronidase (60 U/ml) (Sigma-Aldrich) in PBS at 37° C. for 60 minutes. Cells were filtered through a 70 μm cell strainer and washed with serum containing media. Blood was incubated with lysis buffer for 4 minutes and washed with serum containing media. Spleens were mashed, filtered through a 70 μm cell strainer, incubated with lysis buffer for 4 minutes and washed with serum containing media. Bone marrow was flushed out of the femur with PBS, filtered through a 70 μm cell strainer, incubated with lysis buffer for 30 seconds and washed with serum containing media.

### Flow Cytometry

Single cell suspensions were stained with the following monoclonal antibodies: anti-CD11b (clone M1/70), anti-F4/80 (clone BM8); anti-CD11c (clone N418), anti-CD45 (clone 30-F11), anti-Ly6C (clone AL-21), and a lineage cocktail (Lin) containing anti-CD90.2 (clone 53-2.1), anti-Ter119 (clone TER119), anti-NK1.1 (clone PK136), anti-CD49b (clone DX5), anti-CD45R (clone RA3-6B2) and anti-Ly6G (clone 1A8). The contribution of newly made cells to different populations was determined by in vivo labeling with 5-Bromo-2′-deoxy-uridine (BrdU). Anti-BrdU antibodies were used according to the manufacturer's protocol (BD APC-BrdU Kit). Macrophages were identified as CD45+, CD11 bhi, Lin−/low, CD11clo and F4/80hi. Ly6Chi monocytes were identified as CD45+, CD11bhi, Lin−/low, CD11clo and Ly6Chi. Data were acquired on an LSRII flow cytometer (BD Biosciences), and the data were analyzed using FlowJo v0.0.7 (Tree Star).

### Histology and Immunohistochemistry

Tissues for histological analyses were collected and fixed in formalin and embedded in paraffin. Mouse aortic roots were sectioned into 4 μm slices, generating a total of 90-100 cross-sections per aortic root. Eight cross-sections were stained with hematoxylin and eosin (H&E) and used for atherosclerotic plaque size measurement. Sirius red staining was used for analysis of collagen content. For immunohistochemical staining, mouse aortic roots and human carotid endarterectomy (CEA) sections were deparaffinized, blocked using 4% FCS in PBS for 30 minutes and incubated in antigen-retrieval solution (DAKO) at 95° C. for 10 minutes. Mouse aortic root sections were immunolabeled with rat anti-mouse Mac3 monoclonal antibody (1:30, BD Biosciences). Both mouse aortic roots and CEA samples were stained for prosaposin using a rabbit anti-human prosaposin primary antibody (1:500, Abeam) in combination with a biotinylated goat anti-rabbit secondary antibody (1:300, DAKO). CEA samples were stained for macrophages using a donkey anti-mouse CD68 primary antibody (1:300, Abcam) in combination with a biotinylated donkey anti-mouse secondary antibody (1:300; Jackson ImmunoResearch) Antibody staining was visualized by either Immpact AMEC red (Vectorlabs) or diaminobenzidine (DAB). Sections were analyzed using a Leica DM6000 microscope (Leica Microsystems) or the VENTANA iScan HT slide scanner (Ventana).

### Laser Capture Microdissection

Laser capture microdissection was performed on 24 aortic root sections (6 μm). Frozen sections were dehydrated in graded ethanol solutions (70% twice, 95% twice, 100% once), washed with diethyl pyrocarbonate (DEPC)-treated water, stained with Mayer's H&E and cleared in xylene. For every 8 sections, 1 section was used for CD68 staining (Abd Serotec, 1:250 dilution), which was used to guide the laser capture microdissection. CD68-rich areas within the plaques were identified and collected using an ArcturusXT LCM System.

### RNA Sequencing

The CD68+ cells collected by laser capture microdissection were used for RNA isolation (PicoPure RNA Isolation Kit, Arcturus) and subsequent RNA amplification and cDNA preparation according to the manufacturers protocols (Ovation Pico WTA System, NuGEN). The quality and concentration of the collected samples were measured using an Agilent 2100 Bioanalyzer. For RNA sequencing, pair-end libraries were prepared and validated. The purity, fragment size, yield, and concentration were determined. During cluster generation, the library molecules were hybridized onto an Illumina flow cell. Subsequently, the hybridized molecules were amplified using bridge amplification, resulting in a heterogeneous population of clusters. The data set was obtained using an Ilumina HiSeq 2500 sequencer.

### Cell Proliferation ELISA

For the quantification of cell proliferation, a colorimetric immunoassay based on the incorporation of BrdU during DNA synthesis (Roche, Switzerland) was used. RAW264.7 cells were seeded into 96-well Clear Flat Bottom culture plates (Falcon) at 2.5×103 cells per well and left to adhere overnight. Adhered cells were incubated for 24 hours with either mTORi or S6K1i. Following incubation, BrdU labeling solution was added (1:1000) to each well and left to incubate for 2 hours at 37° C. Following the manufacturer's instructions, the cells were fixed and incubated with Anti-BrdU POD for 1.5 hours. After addition of a substrate solution, the absorbance of the samples was measured at 450 nm with a GoMax-Multi+ plate reader (Promega).

### Metabolic Extra Cellular Flux Analysis

BMDMs were plated at 2.5×103 cells/well in an XF-96-cell culture plate (Seahorse Bioscience) and left to adhere. BMDMs were incubated with either mTORi or S6K1i for 16 hours. The oxygen consumption rate (OCR) was measured in a XF-96 Flux Analyzer (Seahorse Bioscience). The responses to oligomycin, Carbonyl cyanide-4-(trifluoromethoxy)phenylhydrazone (FCCP), and rotenone additions were used to calculate all respiratory characteristics. On completion, DNA content was measured with CyQuant to compensate for differences in cell numbers.

### Preparation of Oxidized LDL

LDL was isolated using KBr-density gradient ultracentrifugation from serum from healthy volunteers. Plasma density was adjusted to d=1.100 g/mL with KBr. The samples were centrifuged for 22 h at 32.000 rpm in a SW41 Ti rotor. Oxidized LDL was prepared by incubation of LDL with 20 μmol CuSO4/L for 15 h at 37° C. in a shaking water bath as described previously. (Tits et al., 2011)

### Human PBMC and Monocyte Isolation

PBMC isolation was performed by dilution of blood in pyrogen-free PBS and differential density centrifugation over Ficoll-Paque. Cells were washed three times in PBS. Percoll isolation of monocytes was performed as previously described (Repnik et al., 2003). Briefly, 150-200-106 PBMCs were layered on top of a hyper-osmotic Percoll solution (48.5% Percoll, 41.5% sterile H2O, 0.16M filter sterilized NaCl) and centrifuged for 15 minutes at 580 g. The interphase layer was isolated and cells were washed once with cold PBS. Cells were resuspended in RPMI culture medium supplemented with 50 μg/ml gentamicin, 2 mM glutamax, and 1 mM pyruvate and counted using a Beckman Coulter counter. An extra purification step was added by adhering Percoll isolated monocytes to polystyrene flat bottom plates (Corning, N.Y., USA) for 1 h at 37° C.; subsequently a washing step with warm PBS was performed to yield maximal purity. (This increases purity to only 3% T cell contamination as described in Bekkering et al., 2016)

### Monocyte Training and Inhibition Experiments

Human monocytes were trained as described before (Bekkering et al., 2016). Briefly, 100,000 cells were added to flat-bottom 96-well plates. After washing with warm PBS, monocytes were incubated either with culture medium only as a negative control, 2 μg/mL β-glucan, 10 μg/ml oxLDL or 10-5000 ng/ml prosaposin for 24 h (in 10% pooled human serum). Cells were washed once with 200 μl of warm PBS and incubated for 5 days in culture medium with 10% pooled human serum, and medium was refreshed once. Cells were re-stimulated with either 200 μl RPMI, LPS 10 ng/ml, or Pam3Cys 10 μg/ml. After 24 h, supernatants were collected and stored at −20° C. until cytokine measurement. In some experiments, cells were pre-incubated (before oxLDL training) for 1 h with nanobiologics (rHDL as a control or 1 μM mTORi-HDL or 0. μM S6K1i-HDL). The training stimuli were added after 1 hour to the cells and inhibitors, leaving the inhibitors on for the remaining training period. After 24 h, both stimuli and inhibitors were washed away and cells were let to rest for 5 days as described above.

### Cytokine and Lactate Measurements

Cytokine production was determined in supernatants using commercial ELISA kits for human TNFα and IL-6 following the instructions of the manufacturer.

RNA Isolation and qPCR

For qRT-PCR, monocytes were trained as described above but with adaption of amounts of cells needed for RNA extraction. 500.000 cells/well were seeded in duplicate in 24-well plates. At day 0 (after 1-hour adherence and washing), day 1 (after training and washing), day 2, day 3 and at day 6, the supernatant was removed and cells were stored in TRIzol reagent. Total RNA purification was performed according to the manufacturer's instructions. RNA concentrations were measured using NanoDrop software, and isolated RNA was reverse-transcribed using the iScript cDNA Synthesis Kit according to the manufacturer's instructions. qPCR was performed using the SYBR Green method. Measured genes are: 18S and prosaposin. Samples were analyzed following a quantitation method with efficiency correction, and 18S was used as a housekeeping gene. Relative mRNA expression levels of non-primed samples at day 0 were used as reference.

### Quantification and Statistical Analysis

### RNA Sequencing Analysis

The pair-ended sequencing reads were aligned to human genome hg19 using TopHat aligner (bowtie2)(Langmead and Salzberg, 2012). Next, HTSeq (Anders et al., 2015) was used to quantify the gene expression at the gene level based on GENCODE gene model release 22 (Mudge and Harrow, 2015). Gene expression raw read counts were normalized as counts per million using trimmed mean of M-values normalization method to adjust for sequencing library size difference among samples. DE genes between drug treatments and control were identified using the Bioconductor package limma (Ritchie et al., 2015). In order to correct the multiple testing problem, limma was used to calculate statistics and P values in random samples after a permutation of labels. This procedure was repeated 1,000 times to obtain null t-statistic and P value distribution for estimating the false discovery rate (FDR) values of all genes. The DE genes of cells isolated from the aortic plaques were identified using a cut-off at a corrected P value of less than 0.2. A cut-off at a corrected P value of less than 0.05 was used to identify the DE genes of RAW264.7 cells. A weighted gene co-expression analysis was constructed to identify groups of genes (modules) involved in various activated pathways following a previous described algorithm(Zhang and Horvath, 2005). In short, Pearson correlations were computed between each pair of genes yielding a similarity (correlation) matrix (sij). Subsequently a power function (aij=Power (sij, β)≡|sij|β), was used to transform the similarity matrix into an adjacency matrix A [aij], where aij is the strength of a connection between two nodes (genes) i and j in the network. For all genes the connectivity (k) was determined by taking the sum of their connection strengths with all other genes in the network. The parameter was chosen by using the scale-free topology criterion, such that the resulting network connectivity distribution approximated scale-free topology. The adjacency matrix was then used to define a measure of node dissimilarity, based on the topological overlap matrix. To identify gene modules, we performed hierarchical clustering on the topological overlap matrix. Subsequently, modules were analyzed with the online annotation tools David (https://david.ncifcrf.gov/) and Revigo (http://revigo.irb.hr/). The DE genes were also mapped to the Kyoto Encyclopedia of Genes and Genomes (KEGG) pathway with KEGG Mapper.

### Statistical Analysis

Results of in vivo experiments are expressed as the mean±SD. Significance of differences were calculated using non-parametric Mann-Whitney U tests and Kruskal-Wallis tests.

In vitro human monocyte experiments were performed at least 6 times and normality checks were performed using visual analysis of histograms and boxplots and a normality assay using Graphpad Prism. Non-parametric parameters were analyzed pairwise using a Wilcoxon signed-rank test. Data are shown as means±SEM.

A p-value below 0.05 was considered statistically significant. All data were analyzed using Graphpad prism 5.0. *p<0.05, **p<0.01, ***p<0.001, ****p<0.0001

### Example 20—Prodrug—General Materials and Methods

All chemicals were purchased from Sigma Aldrich, Medchem Express or Selleckchem, PES syringe filters were obtained from Celltreat. A NE-1002X model microfluidic pump from World precision instruments was used in combination with Zeonor herringbone mixers from Microfluidic-chipshop (#14-1038-0187-05). Particles were purified using a 100 kDa MWCO 20 mL Vivaspin centrifugal filter. Dialysis bags were from Thermo Scientific. The ApoA-I protein was purified in house using a literature procedure xx. Spectroscopic quantification of ApoA-I was performed on a BioTek Cytation 3 imaging plate reader using the Bradfort assay. DLS and Zeta potential measurements were performed on a Brookhaven instrument corporation ZetaPals analyzer, the mean of the number distribution was taken to determine particles sizes. 1H and ¹C NMR samples were analyzed using a Bruker 600 ultrashield magnet connected to a Bruker advance 600 console, data was processed using Topspin version 3.5 μl 7.

Quantitative analysis of all drugs, except dimethylmalonate and its derivatives, was performed by HPLC analysis using a Shimadzu UFLC apparatus equipped with either a C18 or CN column. Acetonitrile and water were used as mobile phase and compounds were detected with an SPD-M20a diode array detector. Dimethylmalonate was analyzed using an Agilent tech 5977B MSD 7890B GC-MS, equipped with a HP5MS 30 m, 0.25 mm, 0.25 m column. Aliphatic and cholesterol derivatized malonate were analyzed using a Waters acquity UPC2 SFC-MS using an isopropanol/water mixture as mobile phase and a 1-aminoantracene column. Radiolabeling of the nanoparticles was performed using a procedure previously reported by us.

### Example 21—Synthesis of the Prodrug—Malonate Derivative

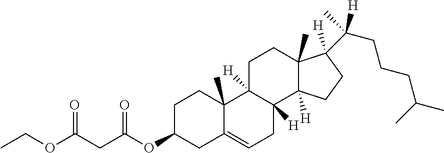 [SMILES: CCOC(=O)CC(=O)O[C@H]1CC[C@@]2(C)C(=CC[C@H]3[C@@H]4CC[C@H]([C@H](C)CCCC(C)C)[C@@]4(C)CC[C@@H]32)C1]

(3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-ylethyl malonate

Cholesterol (194 mg, 0.50 mmol) was dissolved in DCM (30 mL), pyridine (60 μL, 0.75 mmol) was added and the mixture was cooled to 0° C. Ethyl 3-chloro-3-oxopropanoate (80 μL, 0.75 mmol) was dropwise added and the mixture was stirred for 2 hours at 0° C., allowed to warm to room temperature and stirred for an additional 16 hours. Water (60 mL) was added, the layers separated and the aqueous phase was washed twice with DCM (50 mL). The combined organic fractions were dried using MgSO₄ and under vacuum. The crude product was purified using column chromatography (hexane:ethylacetate 1:1) to yield the product as a yellowish solid. Yield: 243 mg, 49 mmol. η=97%. ¹H NMR (600 MHz, CDCl₃) δ=5.41 (br, 1H), 4.69 (m, 1H), 4.22 (q, J=7.1 Hz, 2H), 3.37 (s, 2H), 2.37 (m, 2H), 2.1-1.1 (m, 26H), 1.30 (t, J=7.2 Hz, 3H), 1.03 (s, 3H), 0.92 (d, J=6.5 Hz, 3H), 0.87 (dd, J=6.5, 2.6 Hz, 6H), 0.69 (s, 3H). ¹³C NMR (150 MHz, CDCl₃) δ=166.88, 166.20, 139.52, 123.07, 75.40, 61.61, 56.85, 56.30, 50.17, 42.48, 42.16, 39.89, 39.70, 38.05, 37.09, 36.74, 36.36, 35.97, 32.07, 32.02, 28.41, 28.19, 27.76, 24.46, 24.01, 23.01, 22.75, 21.21, 19.48, 18.90, 14.28, 12.04. Mass calc. for C32H5204=500.39 D, mass found: 501.67 [M+H+], 369.63 [fragment where the malonate-cholesterol bond is split].

### Example 22—Synthesis of the Prodrug—Ethyl Octadecyl Malonate

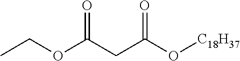

1-octadecanol (250 mg, 1.08 mmol) was dissolved in dry chloroform (30 ml) at 40° C., trimethylamine (165 μL, 119 mmol) was added followed by ethyl 3-chloro-3-oxopropanoate (140 μL, 1.30 mmol). The mixture was stirred for 2 hours, allowed to cool to room temperature and washed with water (3×30 mL). The organic phase was dried using MgSO₄ and under vacuum, the crude product was purified by column chromatography (3% methanol in chloroform) to yield the product as a yellowish wax. Yield=314 mg, 0.82 mmol. η=76%. ¹H NMR (600 MHz, CDCl₃) δ=4.14 (q, J=7.2 Hz, 1H), 4.07 (t, J=6.7 Hz, 1H), 3.30 (s, 2H), 1.61-1.44 (m, 4H), 1.36-1.01 (m, 30H), 1.21 (t, J=7.2 Hz, 6H), 0.81 (t, J=6.8 Hz, 1H). 13C NMR (150 MHz, CDCl₃) δ=166.77, 65.84, 61.65, 41.85, 32.10, 29.87, 29.74, 29.68, 29.54, 29.38, 28.63, 25.96, 22.86, 14.28. Mass calc. for C₂₃H₄₄O₄=384.32 D, mass found. 386 [M+H⁺], 408 [M+Na⁺].

### Example 23—Synthesis of the Prodrug—GSK-J1-Cholesterol

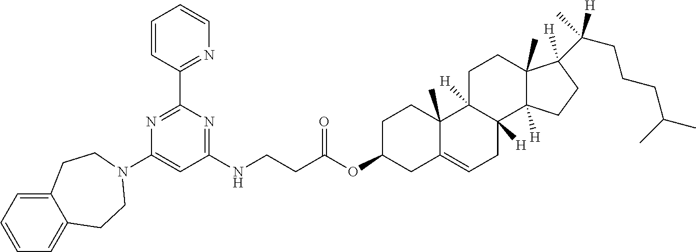 [SMILES: CC(C)CCC[C@@H](C)[C@H]1CC[C@H]2[C@@H]3CC=C4C[C@@H](OC(=O)CCNc5cc(N6CCc7ccccc7CC6)nc(-c6ccccn6)n5)CC[C@]4(C)[C@H]3CC[C@]12C]

(3S,8S,9S,10R,13R,14S,17R)-10,13-dimethyl-17-((R)-6-methyheptan-2-yl)-2,3,4,7,8,9,10,11,12,13,14,15,16,17-tetradecahydro-1H-cyclopenta[a]phenanthren-3-yl-3-((2-(pyridin-2-yl)-6-(1,2,4,5-tetrahydro-3H-benzo[d]azepin-3-yl)pyrimidin-4-yl)amino)propanoate

GSK-J1 (25 mg, 64.2 μmol) was dissolved in dry chloroform (3 mL), EDC.HCl (16.0 mg, 83.3 μmol) and 4-(dimethylamino)pyridine (2.3 mg, 18.8 μmol) were added and the mixture was stirred for 30 min. Cholesterol (27 mg, 69.8 μmol) was added and the mixture was stirred overnight at room temperature. The mixture was washed with water (3×5 mL) and dried using MgSO₄ and under vacuum. The crude product was purified using preparative TLC (6% methanol in chloroform) to yield the product as a white solid. Yield=17.2 mg, 22.7 μmol. η=35%. ¹H NMR (600 MHz, CDCl₃) δ=8.75 (b, 1H), 8.45 (d, J=7.3, 1H), 7.83 (b, 1H), 7.36 (b, 114), 7.15 (s, 4H), 5.57 (s, 1H), 5.36 (b, 14), 4.64 (m, 1H), 3.95 (b, 4H), 3.63 (q, J=6.2 Hz, 2H), 3.03 (m, 4H), 2.65 (t, J=6.4, 2H), 2.33 (d, J=7.5 Hz, 2H), 2.1-1.0 (m, 26H), 1.01 (s, 3H), 0.92 (d, J=6.5 Hz, 3H), 0.86 (dd, J=6.6, 2.7 Hz, 6H), 0.67 (s, 3H). ¹³C NMR (150 MHz, CDCl₃) δ=171.45, 163.60, 162.45, 161.40, 155.17, 149.88, 140.95, 139.68, 137.02, 130.19, 126.67, 124.83, 123.74, 122.96, 79.68, 74.77, 56.86, 56.31, 50.18, 47.68, 42.49, 39.90, 39.70, 38.29, 37.80, 37.14, 37.07, 36.76, 36.37, 35.97, 34.63, 32.08, 29.90, 28.41, 28.20, 27.96, 24.47, 24.01, 23.02, 22.76, 21.21, 19.48, 18.90, 12.04. Mass calc. for C₄₉H₆₇N₅O₂=757.53 D, mass found. 758.77 [M+H⁺], 1516.27 [2M+H⁺].

### Example 24—Synthesis of the Prodrug—GSK-J1-Octadecyl

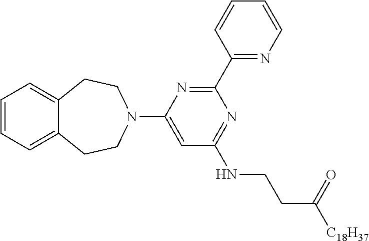

octadecyl 3-((2-(pyridin-2-yl)-6-(1,2,4,5-tetrahydro-3H-benzo[d]azepin-3-yl)pyrimidin-4-yl)amino)propanoate

GSK-J1 (20 mg, 51.4 μmol) was dissolved in dry chloroform (3 mL), EDC.HCl (12.8 mg, 66.6 μmol) and 4-(dimethylamino)pyridine (1.8 mg, 14.8 μmol) were added and the mixture was stirred for 30 min. 1-octadecanol (15.4 mg, 66.6 μmol) was added and the mixture was stirred overnight at room temperature. The mixture was washed with water (3×5 mL) and dried using MgSO₄ and under vacuum. The crude product was purified using preparative TLC (6% methanol in chloroform) to yield the product as a white solid. Yield=19.3 mg, 30.9 μmol. η=60%. ¹H NMR (600 MHz, CDCl₃) δ=8.75 (s, 1H), 8.45 (d, J=7.7 Hz, 1H), 7.81 (t, J=7.1 Hz, 1H), 7.35 (b, 1H), 7.15 (s, 4H), 5.55 (s, 1H), 5.42 (b, 1H), 4.10 (t, J=6.8 Hz, 2H), 3.95 (s, 4H), 3.63 (q, J=6.4 Hz, 2H), 3.05-3.00 (m, 4H), 2.66 (t, J=6.6 Hz, 2H), 1.62 (dt, J=14.7, 6.8 Hz, 4H), 1.37-1.13 (m, 28H), 0.88 (t, J=7.0 Hz, 3H). ¹³C NMR (150 MHz, CDCl₃) δ=172.13, 163.74, 162.54, 156.41, 149.39, 141.03, 136.80, 130.17, 126.64, 124.48, 123.60, 120.07, 79.65, 65.29, 47.64, 37.74, 37.09, 34.36, 32.11, 29.89, 29.79, 29.71, 29.55, 29.46, 28.77, 26.11, 22.88, 14.32. Mass calc. for C₄₀H₅₉N₅O₂=641.47 D, mass found. 642.73 [M+H⁺].

### Example 25—Synthesis of the Prodrug—(+)JQ-1

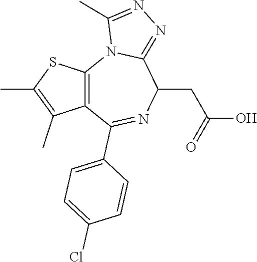 [SMILES: Cc1sc2c(c1C)C(c1ccc(Cl)cc1)=NC(CC(=O)O)c1nnc(C)n1-2]

(S)-2-(4-(4-chlorophenyl)-2,3,9-trimethyl-6H-thieno[3,2-f][1,2,4]triazolo[4,3-a][1,4]diazepin-6-yl)acetic acid

(+)-JQ1 (90 mg, 0.20 mmol) was dissolved in 5% TFA in chloroform (5 mL) and stirred for 16 hours at 40° C. after which the solvent was evaporated. Chloroform (5 mL) was added and evaporated under vacuum, this was repeated twice to yield the product which was used without further characterization. Yield=78 mg, 0.20 mmol. p=>99%.

### Example 26—Synthesis of the Prodrug—(+)JQ-1-Octadecyl

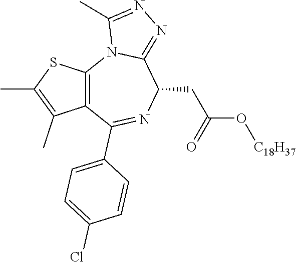

octadecyl (S)-2-(4-(4-chlorophenyl)-2,3,9-trimethyl-6H-thieno[3,2-f][1,2,4]triazolo[4,3-a][1,4]diazepin-6-yl)acetate

(S)-2-(4-(4-chlorophenyl)-2,3,9-trimethyl-6H-thieno[3,2-f][1,2,4]triazolo[4,3-a][1,4]diazepin-6-yl)acetic acid (78 mg, 0.20 mmol) was dissolved in dry chloroform (5 m), EDC.HCl (45 mg, 0.23 mmol) and 4-(dimethylamino)pyridine (37 mg, 0.30 mmol) were added and the mixture was stirred for 30 minutes. 1-octadecanol (63 mg, 0.23 mmol) was added and the mixture was stirred for 16 hours at room temperature. The mixture was washed with water (3×5 mL) and dried using MgSO₄ and under vacuum. The crude product was purified using preparative TLC (6% methanol in chloroform) to yield the product as a white wax. Yield=40 mg, 61 μmol. η=31%. ¹H NMR (600 MHz, CDCl₃) δ=7.40 (d, J=8.2 Hz, 2H), 7.32 (d, J=8.6 Hz, 2H), 4.60 (m, 1H), 4.16 (t, J=6.7 Hz, 2H), 3.65-3.59 (m, 2H), 2.67 (s, 3H), 2.41 (s, 3H), 1.74 (s, 3H), 1.73-1.62 (m, 2H), 1.39-1.32 (m, 2H), 1.32-1.17 (m, 28H), 0.87 (t, J=6.9 Hz, 3H). ¹³C NMR (150 MHz, CDCl₃) δ=171.87, 163.91, 155.57, 150.05, 136.92, 136.79, 132.45, 131.04, 130.87, 130.54, 130.01, 128.85, 65.15, 53.99, 37.08, 32.11, 29.89, 29.81, 29.75, 29.55, 29.49, 28.85, 26.13, 22.88, 14.60, 14.32, 13.29, 12.06.

Mass calc. for C₃₇H₅₃ClN₄O₂S=652.36 D, mass found=653.6 [M+H⁺].

### Example 27—Synthesis of the Prodrug—(+)JQ-1-Cholesterol

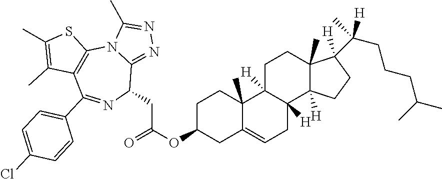 [SMILES: Cc1sc2c(c1C)C(c1ccc(Cl)cc1)=N[C@@H](CC(=O)O[C@H]1CC[C@@]3(C)C(=CC[C@H]4[C@@H]5CC[C@H]([C@H](C)CCCC(C)C)[C@@]5(C)CC[C@@H]43)C1)c1nnc(C)n1-2]

(3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 2-((S)-4-(4-chlorophenyl)-2,3,9-trimethyl-6H-thieno[3,2-f][1,2,4]triazolo[4,3-a][1,4]diazepin-6-yl)acetate

(S)-2-(4-(4-chlorophenyl)-2,3,9-trimethyl-6H-thieno[3,2-f][1,2,4]triazolo[4,3-a][1,4]diazepin-6-yl)acetic acid (75 mg, 0.19 mmol) was dissolved in dry chloroform (5 m), EDC.HCl (50 mg, 0.26 mmol) and 4-(dimethylamino)pyridine (40 mg, 0.33 mmol) were added and the mixture was stirred for 30 minutes. Cholesterol (92 mg, 0.23 mmol) was added and the mixture was stirred for 16 hours at room temperature. The mixture was washed with water (3×5 mL) and dried using MgSO₄ and under vacuum. The crude product was purified using preparative TLC (6% methanol in chloroform) to yield the product as a white powder. Yield=30 mg, 39 μmol. P=21%. ¹H NMR (600 MHz, CDCl₃) δ=7.40 (d, J=8.3 Hz, 2H), 7.32 (d, J=8.6 Hz 2H), 5.36 (d, J=4.1 Hz, 1H), 4.69 (m, 1H), 4.60 (t, 1H), 3.59 (t, J=6.5 Hz, 2H), 2.67 (s, 3H), 2.41 (s, 3H), 2.36 (d, J=6.9 Hz, 2H), 2.1-0.9 (m, 19H), 1.68 (s, 3H), 1.03 (s, 3H), 0.91 (d, J=6.5 Hz, 3H), 0.87 (m, 3H), 0.68 (s, 3H). ¹³C NMR (150 MHz, CDCl₃) δ=171.21, 163.87, 155.58, 150.03, 139.81, 136.91, 136.80, 132.47, 131.02, 130.87, 130.54, 130.00, 128.87, 122.84, 74.70, 56.89, 56.32, 54.08, 50.23, 42.50, 39.93, 39.70, 38.28, 37.29, 37.22, 36.81, 36.37, 35.97, 32.10, 32.03, 29.89, 28.03, 24.47, 24.01, 23.01, 22.75, 21.23, 19.52, 18.91, 14.58, 13.30, 12.05. Mass calc. for C₄₆H₆₁ClN₄O₂S=768.42 D, mass found=769.82 [M+H⁺].

### Example 28—Synthesis of the Prodrug—Rapamycin Prodrug-C17H35

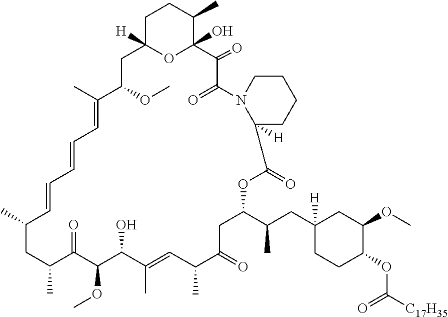

### Rapamycin-C18 Synthesis

Rapamacyin (100 mg, 110 μmol) and vinylstereate (170 mg, 548 μmol) were dissolved in dry toluene (40 mL) and Novozyme 435 (50 mg) was added. The mixture was stirred on a rotavapor at 45° C. for 3 days under mild vacuum. When necessary extra toluene was added. The Novozyme beads were filtered off, the solvent evaporated and the crude product purified using column chromatography (0-6% MeOH in chloroform), to yield the pure product. Yield=108 mg, 89.4 μmol. η=84%. Conversion was monitored by ¹H NMR (600 MHz, CDCl₃) through monitoring of the signal corresponding to the proton adjacent to the alcohol group being esterified, which is present at 2.73 ppm and 4.67 ppm in the unfunctionalized and functionalized Rapamcyin respectively. Mass calc. for C₆₉H₁₁₃NO₁₄ 1179.82 D, mass found 1131.0 [M-OCH₃—H₂O], 1149.0 [M-OCH₃], 1203.0 [M+Na⁺] D (A similar fragmentation pattern was observed for unfunctionalized Rapamycin). Purity was further confirmed by HPLC and TLC.

### Example 29—Synthesis of the 35 nm Nanobiologics

From 10 mg/ml stock solutions in chloroform, 1-palmitoyl-2-oleoyl-sn-glycero-3-phosphocholine (POPC, 250 μL), 1-palmitoyl-2-hydroxy-sn-glycero-3-phosphocholine (PHPC, 65 μL), cholesterol (15 μL), tricaprylin (1000 μL) and (pro-)drug (65 μL), were combined in a 20 ml vial and dried under vacuum. The resulting film was redissolved in a acetonitrile:methanol mixture (95%: 5%, 3 mL total volume). Separately, a solution of ApoA-I protein in PBS (0.1 mg/ml) was prepared. Using a microfluidic set-up, both solutions were simultaneously injected into a herringbone mixer, with a flow rate of 0.75 ml/min for the lipid solution and a rate of 6 ml/min for the ApoA-I solution. The obtained solution was concentrated by centrifugal filtration using a 100 MWCO Vivaspin tube at 4000 rpm to obtain a volume of 5 mL. PBS (5 mL) was added and the solution was concentrated to 5 mL, again PBS (5 mL) was added and the solution was concentrated to approximately 3 mL. The remaining solution was filtered through a 0.22 μm PES syringe filter to obtain the final nanobiologic solution. To obtain nanobiologics for FACS measurements, 3,3′-Dioactadecyloxacarbocyanine perchlorate (DIO-Cis, 0.25 mg) was added to the acetonitrile solution. To obtain nanobiologics for ⁸⁹Zr labeling, DSPE-DFO (50 μg) was added to the acetonitrile solution (made in house). To scale up the nanobiologic synthesis the above procedure was simply repeated until sufficient amounts were produced.

For the PF-4708671 drug (an S6K1i) less than 1% drug recovery was observed using the above procedure, likely due to its high solubility in water and acetonitrile. To still be able to incorporate this drug in our nanobiologic library, it was integrated using a sonication method. Here, an identical lipid and drug film was formed by drying an acetonitrile solution. To this film PBS (10 mL) containing ApoA-I (2.4 mg) was added and the solution was sonicated in a bath sonicator for 5 minutes. Subsequently, the obtained suspension was sonicated for 30 minutes at 0° C. using a tip sonicator. The obtained clear solution was purified using the same Vivaspin and syringe filter procedure as for the nanobiologics made by microfluidics.

### Example 30—Synthesis of the ˜15 nm Nanobiologics

For the synthesis of the 15 nm sized nanoparticles a similar microfluidic procedure as for the 35 nm sized particles was used. Here, the acetonitrile mixture contained (again from 10 mg/ml stock solutions): POPC (250 μL), PHPC (15 μL), Cholesterol (13 μL). The acetonitrile solution was injected with a rate of 0.75 mL/min. The ApoA-I solution (0.1 mg/mL in PBS) was injected with 3 mL/min. To obtain nanobiologics for FACS measurements, DIO-Cis (0.25 mg) was added to the acetonitrile solution. To obtain nanobiologics for ⁸⁹Zr labeling, DSPE-DFO (50 μg) was added to the acetonitrile solution.

### Example 31—Synthesis of the 65 nm Nanobiologics

For the synthesis of the 65 nm sized nanoparticles a similar microfluidic procedure as for the 35 nm sized particles was used. Here, the acetonitrile mixture contained (again from 10 mg/ml stock solutions): POPC (250 μl), Cholesterol (12 μL), Tricaprylin (1400 μL). The acetonitrile solution was injected with a rate of 0.75 m/min. The ApoA-I solution (0.1 mg/ml in PBS) was injected with 4 m/min. To obtain nanobiologics for FACS measurements, DIO-C₁₈ (0.25 mg) of was added to the acetonitrile solution. To obtain nanobiologics for ⁸⁹Zr labeling, DSPE-DFO (50 μg) was added to the acetonitrile solution.

### Example 32—Synthesis of the 120 nm Nanobiologics

For the synthesis of the 120 nm sized nanoparticles a similar microfluidic procedure as for the 35 nm sized particles was used. Here, the acetonitrile mixture contained (again from 10 mg/ml stock solutions): POPC (100 μl), Cholesterol (10 μL), Tricaprylin (4000 μL). The acetonitrile solution was injected with a rate of 0.75 mL/min. The ApoA-I solution (0.1 mg/ml in PBS) was injected with 1.5 mL/min. To obtain nanobiologics for FACS measurements, DIO-Cis (0.25 mg) of was added to the acetonitrile solution. To obtain nanobiologics for ⁸⁹Zr labeling, DSPE-DFO (50 μg) was added to the acetonitrile solution.

### Example 33—Determination of Particle Size and Dispersity by DLS

An aliquot (10 μL) of the final particle solution was dissolved in PBS (1 mL), filtered through a 0.22 μm PES syringe filter and analyzed by DLS to determine the mean of the number average size distribution. Samples were analyzed directly after synthesis of the particles as well as 2, 4, 6, 8, 10 days afterwards.

FIG. 64 shows size and stability of the 4 different types of nanoparticles developed. To solve the issue with radiolabeling the larger two particles we are also investigating radiolabeling the particles using DFO-functionalized APAO1, instead of the previously used DSPE-DFO. Based on the results obtained with DIO loaded particles, and its good reproducibility, we at the time picked the 35 nm particles for creating the nanobiologic library.

FIG. 65 shows the average size each nanobiologic over the day 10 measurement period, two different batches were analyzed for each type of particle. The average size of all nanobiologics over time is also plotted, showing that their size remains constant over time.

FIG. 66 shows the average dispersity of each nanobiologic over the day 10 measurement period, two different batches were analyzed for each type of particle. The average dispersity of all nanobiologics over time is also plotted, showing that their dispersity remains constant over time.

### Example 34—Recovery and Hydrolysis of the Drugs by HPLC

(Pro-)drug recovery and hydrolysis were determined using the following procedure: an aliquot (200 μL) of the particle solution was dried under vacuum, acetonitrile (600 μL) was added and the suspension was sonicated for 20 minutes. The suspension was centrifuged to precipitate any solids and the remaining solution was analyzed using HPLC; except for the malonate derivatives which were analyzed using SFC-MS, and Dimethylmalonate which was analyzed by GC-MS.

FIG. 67 shows recovery of the (pro-)drugs in the nanobiologics. Two batches of every type of nanobiologic were each analyzed in duplicate. Will measure this again for the in vitro sample.

FIG. 68 shows hydrolysis of the (pro-)drugs in the nanobiologics over time at 4° C. in PBS. Only for the Rapamycin and Cis-Rapamycin loaded nanobiologics hydrolysis was observed, in these cases only hydrolysis of the ester in the macrocycle was observed. Two batches of every type of nanobiologic were analyzed. The hydrolysis of the dimethylmalonate and PF-4708671 loaded nanobiologics was not determined because these drugs respectively had 0% recovery, or do not contain a biohydrolyzable moiety.

### Example 35—Determination of the ApoA-I Recovery

The ApoA-I recovery was determined spectroscopically using the Bradfort assay. The nanobiologic solution (10 μL) and calibration solutions (bare ApoA-I in PBS) were placed in a 96-well plate, Bradfort reagent (150 μL) was added and the mixture was incubated at room temperature for 5 minutes after which the absorbance at 544 nm was measured. The average ApoA-recovery for two different batches of each type of nanobiologic is plotted. All calibration and analyte samples were prepared in duplicate.

FIG. 69 shows the average ApoA-I recovery for two different batches of each type of nanobiologic. All calibration and analyte samples were made in duplicate. We will repeat this for the samples made for the in vitro experiments, the large error bars are likely more a result of the poor reproducibility of the used method than representing differences in the actual ApoA-L recovery.

### Example 36—Determination of Zeta Potential

Samples for Zeta potential analysis were prepared by dissolving an aliquot (50 μL) of the final particle solution in MilliQ water (1 mL) and filtering this through a 0.22 μm PES syringe filter. All samples were analyzed in triplicate.

FIG. 70 shows the Zeta potential of each type of nanobiologic in MilliQ water. Samples were analyzed in triplicate. We will repeat this for the samples made for the in vitro experiments.

### Example 37—Determination of Drug Effluence Under In Vivo-Like Conditions

To compare the stability of the nanobiologics under in vivo-like conditions, the nanoparticles were dialyzed in fetal bovine serum at 37° C. The particle solution (0.5 mL) was placed in a 10 kDa dialysis bag, which was suspended in fetal bovine serum (45 mL) at 37° C. At predetermined time points (0, 15, 30, 60, 120, 360 minutes after synthesis) an aliquot (50 μL) was taken from the dialysis bag. The aliquots were dried under vacuum, acetonitrile (100 μL) was added and the solution was sonicated for 20 minutes, after which the remaining suspension was centrifuged and analyzed by HPLC. The dialysis experiments were performed in duplicate using the same batch of nanobiologics. The obtained kinetic data was fitted using a bi-exponential decay after outliers were removed (depicted in red, 5 out of 144 datapoints) and subsequently normalized using the Y-axis intercept of the fit. In some cases, significant amounts of hydrolysis products were observed. Such hydrolyzed (pro-)drugs were assumed to have already leaked out of the nanobiologic, although not yet diffused out of the dialysis bag. For this reason, they were not included in our calculations of the amount of drug retained in the nanobiologics over time.

FIG. 71 shows release of the Malonate derivatives from the nanobiologic, unfunctionalized dimethylmalonate gave 0% drug recovery and was thus not dialyzed. The nanobiologics in PBS (0.5 mL) were dialyzed in fetal bovine serum (45 mL) at 37° C. using a 10 kDa dialysis bag. Experiments were performed in duplicate. The obtained time dependent drug concentrations were fitted using a bi-exponential decay and subsequently normalized.

FIG. 72 shows release of (+)JQ-1 and its derivatives from the nanobiologic. The nanobiologics in PBS (0.5 mL) were dialyzed in fetal bovine serum (45 mL) at 37° C. using a 10 kDa dialysis bag. Experiments were performed in duplicate. The obtained time dependent drug concentrations were fitted using a bi-exponential decay after outliers (red) were removed and subsequently normalized.

FIG. 73 shows release of GSK-J4 and its derivatives from the nanobiologic. The nanobiologics in PBS (0.5 mL) were dialyzed in fetal bovine serum (45 mL) at 37° C. using a 10 kDa dialysis bag. Experiments were performed in duplicate. The obtained time dependent drug concentrations were fitted using a bi-exponential decay after outliers (red) were removed and subsequently normalized.

FIG. 74 shows release of Rapamycin and its derivative from the nanobiologic. The nanobiologics in PBS (0.5 mL) were dialyzed in fetal bovine serum (45 mL) at 37° C. using a 10 kDa dialysis bag. Experiments were performed in duplicate. The obtained time dependent drug concentrations could not be properly fitted using a bi-exponential decay, instead the data was normalized according to the data points at 0 minutes.

FIG. 75 shows release of PF-4708671 from the nanobiologic. The nanobiologics in PBS (0.5 mL) were dialyzed in fetal bovine serum (45 mL) at 37° C. using a 10 kDa dialysis bag. Experiments were performed in duplicate. The obtained time dependent drug concentrations were fitted using a bi-exponential decay and subsequently normalized.

### Example 38—Radiolabelling for Pet Imaging of Accumulation of Trained Immunity Inhibition Drugs

Referring now to FIG. 76, it shows a graphic illustration of the radioisotope labeling process.

In a non-limiting example, radiopharmaceutical labeling of trained immunity inhibitor drugs/molecules can be achieved through various types of chelators, primarily deferroxamine B (DFO) which can form a stable chelate with ⁸⁹Zr through the 3 hydroxamate groups. Generally, phospholipids are conjugated with a chelator compound, the nanobiologic is prepared with the promoter drug or molecule, and finally, the radioisotope is complexed with the nanobiologic (that already has the chelator attached).

### Protocols

This protocol teaches the modular radiolabeling of nanobiologic compositions described herein with ⁸⁹Zr. This protocol includes the synthesis of DSPE-DFO, obtained through reaction of the phospholipid DSPE and an isothiocyanate derivative of the chelator DFO (p-NCS-Bz-DFO), its formulation into nanobiologics, and nanoemulsions, and the subsequent radiolabeling of these nanoformulations with ⁸⁹Zr.

The radioisotope ⁸⁹Zr was chosen due to its 3.3-day physical decay half-life, which eliminates the need for a nearby cyclotron and allows studying agents that slowly clear from the body, such as antibodies. Although both are contemplated as workable herein, ⁸⁹Zr's relatively low positron energy allows a higher imaging resolution compared to other isotopes, such as ¹²⁴I.

The ⁸⁹Zr labeling of our nanotherapeutics enables non-invasive study of in vivo behavior by positron emission tomography (PET) imaging in patients.

The protocol includes the following steps: Conjugation of the chelator deferoxamine B (DFO) to the phospholipid DSPE, to thereby form a lipophilic chelator (DSPE-DFO) that readily integrates in different lipid nanoparticle platforms (˜0.5 wt %);

Preparation of nanoscale assembly formulations (using sonication, nanoemulsions using hot dripping, or using microfluidics) that have [symbol] DSPE-DFO incorporated; and

Labeling of DSPE-DFO containing lipid nanoparticles with ⁸⁹Zr, performed by mixing the nanoparticles for 30-60 minutes with ⁸⁹Zr-oxalate at pH-7 and 30-40° C. in PBS.

Additionally, purification and characterization methods may be used to obtain radiochemically pure ⁸⁹Zr-labeled lipid nanoparticles. Purification may typically be performed using either centrifugal filtration or a PD-10 desalting column, and subsequently assessed using size exclusion radio-HPLC. Typically, the radiochemical yield is >80%, and radiochemical purities >95% are normally obtained.

General imaging strategies are used to study ⁸⁹Zr-labeled nanobiologic in vivo behavior by PET/CT or PET/MRI.

FIG. 77 shows PET imaging using a radioisotope delivered by nanobiologic and shows accumulation of the nanobiologic in the bone marrow and spleen of a mouse, rabbit, monkey, and pig model.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

1. A nanobiologic composition for inhibiting trained immunity, comprising:
a nanoscale assembly, having (ii) an inhibitor drug incorporated in the nanoscale assembly,
wherein the nanoscale assembly is a multi-component carrier composition comprising:
(a) a phospholipid or a mixture of phospholipids,
(b) apo AI or a peptide mimetic of apo AI, and
(c) a matrix lipid selected from one or more triglycerides, fatty acid esters, hydrophobic polymers, and sterol esters,
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell.

2. A nanobiologic composition for inhibiting trained immunity, comprising:
a nanoscale assembly, having (ii) an inhibitor drug incorporated in the nanoscale assembly,
wherein the nanoscale assembly is a multi-component carrier composition comprising:
(a) a phospholipid or a mixture of phospholipids,
(b) apo AI or a peptide mimetic of apo AI,
(c) a matrix lipid selected from one or more triglycerides, fatty acid esters, hydrophobic polymers, and sterol esters, and
(d) cholesterol
wherein said nanobiologic, in an aqueous environment, is a self-assembled nanodisc or nanosphere with size between about 8 nm and 400 nm in diameter;
wherein said inhibitor drug is a hydrophobic drug or a prodrug of a hydrophilic drug derivatized with an attached aliphatic chain or cholesterol or phospholipid,
wherein the drug is an inhibitor of the inflammasome, a metabolic pathway or an epigenetic pathway within a hematopoietic stem cell (HSC), a common myeloid progenitor (CMP), or a myeloid cell.

3. The nanobiologic composition of CLAIM 1, wherein the inhibitor of a metabolic pathway or an epigenetic pathway comprises: a NOD2 receptor inhibitor, an mTOR inhibitor, a ribosomal protein S6 kinase beta-I (S6K1) inhibitor, an HMG-CoA reductase inhibitor (Statin), a histone H3K27 demethylase inhibitor, a BET bromodomain blockade inhibitor, an inhibitor of histone methyltransferases and acetyltransferases, an inhibitor of DNA methyltransferases and acetyltransferases, an inflammasome inhibitor, a Serine/threonine kinase Akt inhibitor, an Inhibitor of Hypoxia-inducible factor I-alpha, also known as HIF-I-alpha, and a mixture of one or more thereof.

4. The nanobiologic composition of CLAIM 2, wherein the inhibitor of a metabolic pathway or an epigenetic pathway comprises: a NOD2 receptor inhibitor, an mTOR inhibitor, a ribosomal protein 6 kinase beta-I (S6K1) inhibitor, an HMG-CoA reductase inhibitor (Statin), a histone H3K27 demethylase inhibitor, a BET bromodomain blockade inhibitor, an inhibitor of histone methyltransferases and acetyltransferases, an inhibitor of DNA methyltransferases and acetyltransferases, an inflammasome inhibitor, a Serine/threonine kinase Akt inhibitor, an Inhibitor of Hypoxia-inducible factor I-alpha, also known as HIF-I-alpha, and a mixture of one or more thereof.
